(19) United States
(12) Patent Application Publication
(10) Pub. No.: US 2002/0187105 A1
(43) Pub. Date: Dec. 12, 2002

(54) Polymer combinations that result in stabilized aerosols for gene delivery to the lungs

(76) Inventors: Yiyu Zou, Bronx, NY (US); Roman Perez-Soler, New York, NY (US)

Correspondence Address: FULBRIGHT & JAWORSKI L.L.P., A REGISTERED LIMITED LIABILITY PARTNERSHIP, SUITE 2400, 600 CONGRESS AVENUE, AUSTIN, TX, 78701, US

(21) Appl. No.: 10/061,444

(22) Filed: Feb. 1, 2002

Related U.S. Application Data
(60) Provisional application No. 60/266,174, filed on Feb. 1, 2001.

(51) Int. Cl. A61K 31/785; A61K 38/16; A61L 9/04; A61K 31/77

(52) U.S. Cl. 424/45; 424/78.38; 514/2

(57) ABSTRACT

The use of non-viral delivery of therapeutically effective compositions through aerosol for therapy or research purpose has been limited by the low efficiency mainly caused by an inefficient delivery system and destruction of formulation (gene and/or delivery system) by aerosol shearing power. This invention develops formulations that are established polymer combination formulations. The formulations are highly efficient in delivering genes in vivo through aerosol and are able to protect the delivered gene from the destruction by aerosol shearing power.

### BACKGROUND OF THE INVENTION

This application claims benefit of the filing date of U.S. Provisional Patent Application Serial No. 60/266,174 filed on Feb. 1, 2001. The entire text of the above-referenced disclosure is specifically incorporated by reference herein in its entirety without disclaimer.

A. Field of the Invention

The present invention relates generally to the fields of aerosol delivery. More particularly, it concerns a novel formulation for the efficient delivery of genes or other pharmaceutically acceptable agents in vivo that protects the pharmaceutically acceptable agent from destruction by aerosol shearing forces. The present invention also concerns methods of preparation of such compositions and methods of transfecting cells with such compositions.

B. Description of Related Art

The success of gene therapy is largely dependent on the development of a vector or vehicle that can selectively and efficiently deliver a gene to target cells with minimal toxicity. Viruses are efficient in transducing cells, and thus constitute a popular choice as a delivery vehicle in gene therapy.

In the case of viral components, it is usually replication incompetent or attenuated viruses that are used. Unfortunately, the viral genome is still capable of low level expression of viral proteins such as the major hexon coat protein (Yang et al., 1994). This expression occurs at sufficient levels to induce an immune response, which has resulted in continued problems with immunogenicity as well as toxicity (Yang et al., 1994). It has also become apparent that the vector itself is immunogenic and that this immune response may never be overcome in developing future gene therapy delivery compositions based on this virus (Kafri et al., 1998). Overall, it has become apparent that this viral delivery composition/vector is immunogenic, difficult to produce economically in large quantities, has a limited therapeutic nucleic acid carrying capacity, a continued dependence upon helper cell lines for production, a lack of targeting, and is still plagued by questions related to safety and toxicity (Marshall, 1999).

These safety concerns regarding the use of virus in humans make non-viral delivery systems an attractive alternative. Non-viral vectors are particularly suitable with respect to simplicity of use, ease of large-scale production and lack of specific immune response. The three most commonly investigated non-viral delivery composition components are based on formulations involving lipids (e.g., liposomes) (Bendas et al., 1999), polycations (Xu et al., 1998), or simple naked DNA (Chen et al., 2000). Unfortunately, delivery compositions containing these components have traditionally had recurrent problems of low transduction efficiency particularly in vivo; naked DNA exhibits the lowest and liposomes exhibit the highest (Bendas et al., 1999; Xu et al., 1998; Chen et al., 2000). Other problems with these delivery compositions include toxicity of the delivery formulation.

While gene expression can be achieved by direct intratissue injection of naked plasmid DNA, gene transfer via other routes of administration such as intratracheal and intravenous injection and aerosol generally require the use of a delivery vector or vehicle.

Since Felgner et al. first described a successful in vitro transfection with cationic lipid in 1987, there has been substantial progress in the application of synthetic lipids for use in gene delivery systems. Novel cationic lipids have been synthesized (Wheeler et al.; 1996, Lee et al., 1996) and used for in vitro and in vivo transfection.

Cationic lipid/DNA complexes (lipoplexes) have been used in several clinical trails for the treatment of cancer and cystic fibrosis (Nabel et al., 1993; Caplen et al., 1995). Lipoplexes were proven to be safe when locally delivered at relatively low doses. However, no long-term safety studies have been performed. Also, the efficiency of lipidic vectors needs to be improved before cationic lipid-mediated gene transfer can become standard practice in the clinic. Despite progress in the field of lipid delivery compositions, current lipidic vectors are still inefficient in active targeting of genes to specific tissues.

Polycationic polymers lie in the middle of properties regarding ease of delivery composition production and formulation. Polycationic polymers have a self-assembling property when mixed with nucleic acids due to ionic interactions. There have been many studies utilizing the synthetic polycation polyethylenimine (PEI) as a component to deliver nucleic acids to cultured cells as well as to cells in vivo (Bousiff et al., 1995; Boussifet al., 1996; Densmore et al., 2000; Fronsdal et al., 2000; Boletta et al., 1997; Goula et al., 1998; Coll et al., 1999; Kircheis et al., 1997; Hart, 2000; Rudolph et al., 2000).

The utilization of polycationic polymers for delivery of pharmaceutical agents has led to many different applications for these molecules. One group in particular has been termed, “molecular conjugates” (Cristiano and Roth, 1995). Molecular conjugates are composed of cell and delivery composition specific proteins that have been attached to positively charged polycationic polymers. These conjugates bind DNA to form a protein-DNA complex or polyplex (based on the use of polycations) that can target DNA to a specific cell type depending upon the components used.

The mode of delivery of pharmaceutical agents can be important for the effectiveness of the agent. One mode of delivery is by aerosol. Aerosolization is a fast and effective means to transport pharmaceutal agents into the body. Aerosol delivery can be used to directly contact the delivery composition to the lung. In the lung, many different diseases have been treated successfully through utilization of aerosol delivery systems used to deposit drugs directly on to the pulmonary surfaces.

The field of administration of aerosolized therapeutics to the lungs is known. For example, the formulation of complexes for aerosol delivery and apparatus for forming aerosol particles are taught in, for example, U.S. Pat. No. 5,962,429, U.S. Pat. No. 6,090,925, U.S. Pat. No. 5,744,166, U.S. Pat. No. 5,985,309 and U.S. Pat. No. 5,639,441.

Formulations of vectors for aerosol delivery in gene therapy include cationic lipids. The use of cationic lipids based formulations for delivery to and transfection of the lung via aerosol gene delivery has been described in, for example, U.S. Pat. No. 5,641,662, and U.S. Pat. No. 5,756,353. Cationic lipids as part of an aerosol formulation is taugh in, for example, U.S. Pat. No. 6,086,913, U.S. Pat. No. 5,981,501, U.S. Pat. No. 6,106,859, U.S. Pat. No. 6,008,202, Stribling et al., 1992; Crook et al., 1996; Eastman et al., 1997; Chadwick et al, 1997; McDonald et al., 1998; Birchall et al., 2000 and Densmore et al., 2000. However, there are several problems associated with cationic lipid vectors for use in aerosol delivery. One problem is lower transfection efficiency and poor stability of the cationic lipid formulations precludes the use of many cationic lipids as delivery vectors.

A problem often encountered with aerosol delivery is that the aerosolation of the particles causes destruction of the therapeutic efficacy of the agent to be administered. The shear forces created by extrusion of the formulation through the jet orifice of a nebulizer are great enough to reduce the activity of many compounds. Another problem is the low stability of the therapeutic agents being delivered. Often, these agents will lose effectiveness within a few minutes of entering the lungs.

Work has been done in the field of increasing the stability of the particles in the delivery vector formulations. Caponetti et al., 1999; Lee et al., 1997; teach the use of poly-L-lysine (PLL) and include polyethylene glycol (PEG) in alginate-PLL microcapsules to enhances mechanical stability of the particles. Similarly, U.S. Pat. No. 6,008,202, Densmore et al., 1999; Godbey et al., 2000; Vinogradov et al., 1998; Ogris et al., 1999; Eastman et al., 1997; and Gautam et al., 2000 teach of the use and increased stability of several PEI-based formulations. However, the use of PEI based formulations can cause the toxicity of the formulation to increase. Thus, there remains a need for improved lipid-based delivery systems.

### SUMMARY OF THE INVENTION

Thus, in accordance with the present invention, there is provided a method for administering a composition to a cell, membrane, organ or tissue comprising contacting said composition with said cell, membrane, organ or tissue wherein said composition comprises polyethyleneglycol (PEG), a polycationic polymer, polyethylenimine (PEI) and a cationic lipid is provided. This administering may be in the form of an aerosol. The cationic lipid can be any cationic lipid that will help reduce the toxicity of the composition; phospholipids such as dipalmitoyl glycero ethylphosphocholine (DPEPC), other diacy-dimethylammonium propanes such as DSEPC, DMEPC, DLEPC, DOEPC, or palmitoyl-oleoyl-EPC, a diacyl-dimethylammonium propane such as DSDAP, DPDAP, DMDAP, or DODAP, a diacyl-trimethylammonium propane such as DSTAP, DPTAP, DMTAP, or DOTAP are selected. Other cationic lipids include, for example, dimethyldioctadecylammonium (DDAB), N-[1-(2,3-ditetradecyloxy)propyl]-N,N-dimethyl-N-hydroxyethylammonium, bromide (DMRIE), N-[11-(2,3,-dioleyloxy)propyl]-N,N-dimethyl-N-hydroxy ethylammonium, bromide (DORIE ), N-[1-(2,3-dioleyloxy) propyl]-N,N,N-trimethylammonium chloride (DOTMA), DOSPA, 3-beta-[N-(N,N′-dimethylaminoethane)carbanmoly]cholesterol (DC-Chol), 3- beta-[N-(NN-dicarbo-benzoxyspemidine)carbamoyl]cholesterol, or 3-beta-(N-spemine carbamoyl) cholesterol. Particular polycationic polymers include protamine, poly(amino acids) such as polylysine, polyhistidine, or polyarginine, or other cationic polymers such as poly(L-ornithine), poly (dimethylamine) ethyl methacylate, or poly (trimethylamine) ethyl methacylate. More specifically, protamine and polylysine as the selected polycationic polymers. Two, three or more cationic polymers can be incorporated in the formulations of this invention.

An aspect of the current invention is the increased transduction efficiency of the composition. Use of the composition of this invention with a therapeutic agent, wherein the ratio of said composition to said therapeutic agent is no more than 50:1 is preferred. More prefered is a ratio of 10:1. The composition can be used in concentrations of 100:1 compared to the therapeutic agent if needed.

In certain embodiments, it is contemplated that composition further comprises a nucleic acid, DNA, RNA, a protein, a vaccine, an oligonucleotide, an antisense oligonucleotide, an expression construct, a coding region for p53, a chemical agent, an antibiotic, a chemotherapeutic agent or a diagnostic agent. Administration of the composition can be to anywhere in the airways, such as the lungs, the trachea or the alveoli.

In certain embodiments, it is contemplated that the composition is administered to prevent or treat, for example, lung cancer, a lung infection, asthma, bronchitis, emphysema, bronchilitis, cystic fibrosis, bronchiectasis, pulmonary edema, pulmonary embolism, respiratory failure, pulmonary hypertension, pneumonia or tuberculosis. It is preferred that the composition is administered to prevent or treat lung cancer.

A further aspect of the current invention comprises the diameter of particles in the suspension of said pharmaceutical composition in the range of 5-0.01 μm, or more preferrably 2-0.05 μm, or more preferrably 0.05 μm and 0.2 μm. A diameter of 0.01 μm has been found to be ideal for various applications. When referring to a range in the diameter of the particles in the composition of the current invention, it is understood that at least 80% of the particles fall within the range. Some particles or aggregates larger than the prescribed range are expected in the composition. The particles may form a dry powder or a liquid. The preferable aerosol droplet diameter is 0.3-3.0 μm.

Another embodiment of the current invention comprises a method for formulating a composition for aerosol delivery comprising combining polyethyleneglycol (PEG), a polycationic polymer, polyethylenimine (PEI) and a cationic lipid to create a composition wherein said composition is capable of being administered as an aerosol. It is an aspect of the current invention that said composition further comprises a stabilizer or a cosolvent.

It is an aspect of the current invention wherein the ratio of said PEG to said protamine in said composition is from about 1:1 to 1:5 or more preferably about 1:2. The ratio of said PEG to said polylysine in said composition is from about 1:1 to 10:1 or more preferably about 3:2. The ratio of said PEI to said polycationic polymer in said composition is from about 1:5 to 1:20 or more preferably about 1:10. The ratio of said cationic lipid to said polycationic polymer in said composition is from about 1:2 to 1:20 or more preferably from about 1:3 to 1:20. The ratio of said DPEPC to said polycationic polymer in said composition is from about 1:3 to 1:20 or more preferably about 1:5. The ratio of said components protamine, PEG, PEI, and DPEPC in said composition is from about 2:1:1:0.4 to 50:25:1:10 or more preferably about 10:5:1:2. The ratio of said components polylysine, PEG, PEI, and DPEPC in said composition is from 2:3:1:0.4 to 50:80:1:10 or more preferably about 10:16:1:2.

When referring to a ratio, it is understood that a certain amount of error is allowed. For example, a ratio of about 2:1 is understood to include values between 1.90:1 and 2.10:1. Although the prefered ratios are given for the particular components: polylysine, protamine, PEG, PEI, and DPEPC it is understood that other cationic lipids and other polycationic polymers may be used. The ratios for these compositions can be determined without undue experimentation given the information described herein.

Another embodiment of this invention provides a composition for aerosol delivery comprising polyethyleneglycol (PEG), a polycationic polymer, polyethylenimine (PEI) and a cationic lipid. This composition may also comprise an aerosol canister which, in a further embodiment, comprises a means for metering dosages.

A further embodiment of the current invention provides a method for reducing toxicity of polyethylenimine (PEI) in an aerosol formulation wherein dipalmitoyl-glyceroethyl phosphocholine (DPEPC) is added to said aerosol formulation in an amount sufficient to reduce the toxicity of said PEI.

A further embodiment provides a composition for aerosol delivery comprising polyethyleneglycol (PEG), a polycationic polymer and a pharmaceutically active agent, wherein the activity of said pharmaceutically active agent ten minutes after aerosolization is at least 50% of initial activity of said pharmaceutically active agent. It is more preferred that the activity at ten minutes after aerosolization is at least 60% of initial activity of said pharaceutically active agent. It is even more preferred that the activity at ten minutes after aerosolization is at least 70% of initial activity of said pharaceutically active agent. The polycationic polymer can comprise protamine, polylysine, or a combination of polycationic polymers.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1.—Cyto-toxicity for PEI and lipid-PEI combinations as a function of PEI concentration shown as percent cell death measured by Trypan blue staining. The lipid-PEI combination (L-PEI) was obtained by hydrating the lipid thin film of dipalnitoylglyceroethylphosphocholine (DPEPC) with polyethylenimine (PEI). Human normal bronchial epithelial cells (HNBE) were used. The data is mean ±SD from 3 independent studies.

FIG. 2.—Transfection efficiency of a lipid, PEI and a lipid-PEI combination using human non-small cell lung carcinoma cell lines A549, H322, and H358. The cationic lipid (DPEPC liposomes), PEI, and the lipid-PEI combination L-PEI (1:2 w/w) were complexed with green-fluorescence-protein. The data is mean ±SD from 3 independent studies.

FIG. 3.—Transfection efficiency of single (PEI, protamine and polylysine) and multiple cationic polymers formulation in transfection of GFP into human non-small cell lung carcinoma cell lines (H322 and H358). The data is mean ±SD from 3 independent studies.

FIG. 4.—The formulations containing multiple cationic polymers are stable than that of single cationic polymer or liposome formulations during aerosolization. The data is mean ±SD from 3 independent studies.

FIGS. 5A-5D.—Aerosolized formulations of Z1, Z2, Z3, Z4 and lipofectamine (Lf) containing wild-type p53 gene expression plasmid and p21 promoter driven luciferase plasmid.

FIG. 5A. Percent luciferase activity for samples at 0 and 10 minutes after aerosol delivery.

FIG. 5B. Percent luciferase activity remaining at 10 minutes after aerosol delivery.

FIG. 5C. Percent apoptotic cells at 0 and 10 minutes after aerosol delivery.

FIG. 5D. Percent killing of cells; multiple cancer cell lines (A549, H322, H358, and H460). The termination assay used was counting viable cells. The data is mean ±SD from 3 independent studies.

FIG. 6.—|Relative luciferase activity for PEI, Z 1, Z2, Z3 and Z4 formulations at 6 and 10 minutes of aerosol administration in the lungs of mice. The data is mean ±SD from 3 independent studies.

FIGS. 7A-7B.—Efficiency of formulation delivery for mice bearing orthotopic human lung cancer.

FIG. 7A. Efficiency of Z1 and Z4 delivery to various mouse tissues for formulations complexed with luciferase expression plasmid through aerosol administration.

FIG. 7B. Efficiency of Z1 and Z4 delivery to various mouse tissues. The DNA in the aerosolized formulations for FIG. 7B was wild-type p53 gene expression plasmid and p21 promoter driven luciferase plasmid. The data is mean ±SD from 3 independent studies.

FIGS. 8A-8B.—Percent survival of mice bearing orthotopic human lung cancer after administration of aerosolized Z1-, Z4-, and Lf-p53. FIG. 8A. Inoculation was with H358. FIG. 8B. Inoculation was with H322. The data is mean ±SD from 5 independent studies.

### DESCRIPTION OF ILLUSTRATIVE EMBODIMENTS

The current invention relates to a lipid/polymer formulation that, when given as an aerosol, can efficiently deliver genes and other biological products (e.g., genes, RNA, proteins, oligonucleotides) chemical agents (e.g., chemotherapy drugs, antibiotics, other drugs) or diagnostic agents (e.g., gases) to the lungs. A combination of single cationic polymer and cationic lipids, or PEI alone is not efficient for gene delivery through aerosol administration. However, the appropriate combination of PEG and multiple cationic polymer given as an aerosol can efficiently deliver genes to the lungs and to lung cancer cells. The addition of a certain amount of cationic phospholipids can increase the gene delivery efficiency in vivo. Particularly, this invention may be used for therapy or prevention of lung diseases such as lung cancer, cystic fibrosis, TB, lung infection and asthma.

I. Nucleic Acid Delivery Compositions

In certain embodiments, the aerosol delivery formulation of the present invention comprises at least one polycationic polymer, PEG and at least one nucleic acid. In further embodiments, the delivery composition further comprises at least one additional agent, including but not limited to a targeting agent (e.g., a targeting ligand), an endosome agent, a linker/coupling agent, a proteinaceous compound, a lipid, a drug, an anti-cancer agent, a vaccine component, a pharmaceutically acceptable carrier or any combination thereof such agents. In a non-limiting example, a composition of the present invention may comprise a polycationic polymer attached to a linker/coupling agent, which is attached to a targeting agent. In another non-limiting example, a composition of the present invention may comprise a nucleic acid in a liposome which comprises a targeting agent. Of course, other combinations of aerosol delivery formulation components are described herein, and additional combinations will be readily apparent to one of skill in the art from the disclosures herein, and are thus encompassed by the present invention. The various components of an aerosol delivery formulation may be associated to each other by means including, but not limited to, covalent bonds, ionic interactions, hydrophobic interactions or combinations thereof.

In particular embodiments, the aerosol delivery formulation components include multiple cationic polymers, PEG, PEI, and a cationic lipid. A nucleic acid may be purified on polyacrylamide gels, cesium chloride centrifugation gradients, or by any other means known to one of ordinary skill in the art. For example, see Sambrook et al., (1989), incorporated herein by reference, wherein a DNA purification protocol based on a variation of the alkaline lysis procedure is described.

A. Polymers

Polymers or mixtures of polymers can be used to prepare the aerosol formulation of the current invention. Polymers can be used to remove water from or dehydrate nucleic acid compositions or causing volume exclusions. Polymers also possess the advantages of the ability to serve as a point of a binding ligand and/or chemical moiety attachment, such as through a covalent bond.

A particular polymer of the current invention is polyethyleneglycol (PEG), which is also known as poly(oxyethylene) glycol. PEG is a condensation polymer of ethylene oxide and water, and has the general chemical formula HO(CH₂CH₂O)ₙH. Also contemplated in the current invention are branched PEG and derivatives thereof, PEG-acrylates, and other PEG co-polymers.

B. Polycationic Polymers

Polycationic polymers have the advantages of self-assembling when combined with a nucleic acid (e.g., DNA, RNA, PNA or combinations thereof), making them simple to use, and are commercially available, inexpensive and do not require difficult synthesis strategies. It is contemplated that any polycationic polymer described herein or as would be known to one of ordinary skill in the art may be used in the compositions and methods described herein.

Polycationic polymers also possess the advantages of the ability to serve as a point of a binding ligand and/or chemical moiety attachment, such as through a covalent bond. Most importantly, some polycationic polymers possess an ability to function in the role of an endosome lysis agent, and thus can increase the passage of DNA or a pharmaceutically acceptable composition into the cell's cytoplasm. The high number of cationic chemical moieties (e.g., amines) allows the molecule to act as a “proton sponge”, using its cationic moieties to absorb hydrogen ions during the acidification of the endosome which leads to endosome lysis. Polycationic polymers that can serve as endosome agents are preferred in certain embodiments of the present invention.

Examples of polycationic polymers are poly(amino acids) including but not limited to polylysine, polyhistidine, or polyarginine, or other cationic polymers such as protamine, poly(L-ornithine), poly (dimethylamine) ethyl methacylate, or poly (trimethylamine) ethyl methacylate.

In certain embodiments, a polycationic polymer may condense a nucleic acid by electrostatic charge-charge interactions (Plum et al., 1990). For example, the neutralization and condensation of DNA by polycationic polymers, such as polylysines, into small (ca 100 nm) toroid-like structures, promotes the endocytosis of the nucleic acid into cells in vitro (U.S. Pat. No. 5,972,600, incorporated herein by reference). The neutralization of a nucleic acid's negative charge may aid tranfections, as cells surfaces are often negatively charged (Stevenson et al., 1989; Lemaitre et al., 1987). Additionally, polycationic polymers, such as polylysines also destabilize cell membranes, and may be used as a site for the attachment of additional agents (U.S. Pat. No. 6,071,533, incorporated herein by reference).

In certain embodiments, the number of monomers in an individual polycation chain can be of from 3 to about 1000 monomers, and any integer derivable therein and any range derivable therein. Of course, in various aspects mixtures of polycation chains of different lengths can be used. In other embodiments, the number of cationic moieties on a particular polycation chain may comprise of from 3 to about 1000 monomers, and any integer derivable therein and any range derivable therein. In specific aspects, the number of cationic moieties or charges is matched to, or approximates the number of anionic moieties or charges in a nucleic acid, proteinaceous composition, or composition of the present invention.

In certain embodiments, the polycationic polymer is a polyamine, such as, for example, spermidine, spermine, polyammonium molecules such as, for example, polybrene (hexadimethrine bromide), basic polyamino acids (e.g., polylysine), basic proteins or a combination thereof. Other polycationic polymers include, but are not limited to, those described in U.S. Pat. Nos. 5,656,611, 5,354,844, 5,462,866, 5,462,866 and 5,494,682, each incorporated herein by reference.

In other embodiments, the polycationic polymer is a protamine, histone, heterologous polypeptide, non-peptide cations such as polyethyleneimines, or a combination thereof (U.S. Pat. No. 5,792,645, incorporated herein by reference).

In other embodiments, a polycationic polymer may comprise, for example, a cationized albumin, DEAE-dextran, a histone, polybrene, polyornithine, protamine, spermine, a cascade amidoamine “dentritic” polymer, gramicidin S cyclic peptide, spermidine, polylysine, such as, for example, the (bromide salt, mol. wt. 25,600; Sigma Chemical Corporation St. Louis, Mo.), a short, synthetic cationic peptide, or combinations thereof (U.S. Pat. No. 5,908,777; Haensler and Szoka, 1993, each incorporated herein by reference). U.S. Pat. No. 5,260,002 describes various polymers contemplated herein.

In the present embodiment of the invention, it is contemplated that the cationic members of polymers (e.g., gelatin), as would be understood by one of ordinary skill in the art, may be used as a polycationic polymer of the present invention. Such polymers include NIH Approved Implantable materials, including, polyacids such as polyacrylates (e.g., sodium), polymethacrylates and olefin maleic anhydride copolymers; polyesters, such as polyglycolic acid, poly lactic acid, poly caprolactane and copolymers of these polyesters; polyorthoesters, such as polydioxyalkyltetrahydrofuran and poly 3,9-bismethylene-2,4,8,10 tetra aspiro 5,5 undecane-co-1,6 hexanediol; hydrogels, such as, PEG, hydroxyethylmethacrylate, monomethyacrylate and gelatin crosslinked with formaldehyde; polysaccharides such as cellulose and dextran; polypeptides, such as, polyglutamic acid, glutamic acid leucine copolymers, polyaminotriazole/alkyleneaminotriazole copolymers and albumin beads (i.e, albumin crosslinked with glutaraldehyde); amino acid polymers, such as poly D- or L-lysine HCL, poly D- or L-omithine HCL and poly D- or L-arginine; and combinations thereof.

Other polymers described included water soluble polymers such as polysaccharides (−): starch, gums, carrageenans, dextran, xanthan, sulfated algal polysaccharide (−), alginate (−), hyaluronic acid films (−), heparin (−), chondroitin sulfates (−), polygalacturonic acid (−), alginic acid (−), sodium carboxymethylcellulose (−), sodium carboxymethylcellulose-diethylaminoethyldextran copolymer (−), agar, hyaluronate (−), sulfated hyaluronic acid (−), sulfated deacetylated hyaluronic acid (−), heparin (−), polyguluronate (normal or acetylated) (−), polymannuronate (−), chondroitin sulphate (−), ascopyllan (−), pectin (made of 1,4 polyglacteronic acid) (−), dextran sulfate (−), fucoidan (−), oxdized cellulose (−), polypeptides and proteins such as hydrophobic (e.g., polyphenylalanine), polar (e.g., serine), acidic (−) (e.g., asparatic acid, chondroitin-6-sulfate, heparin, human serum albumin, basic (+) (e.g., lysine, 1-argine, collagen); polynucleic acids (RNA, DNA) (nonionic), pullan (nonionic), cellulose (nonionic), algal pectin, modified celluloses such as hydroxypropylcellulose (nonionic, forms a thin film), hydroxypropylcellulose (nonionic), carboxymethylcellulose (nonionic); forms a gel/film, diethylaminohydroxypropylcellulose (+), diethylaminoethylcellulose (+) and chitosan (+).

Other polymers disclosed include synthetic polymers, such as the nonionic polymers polyacrylamide, polymethacrylamide, polyvinyl alcohol films; the anionic polymers poly sodium acrylate, polystyrene sodium sulphate, polyvinyl sulphonic acid salts, polyvinyl benzoic acid salts, polyvinyloxypropanesulphonic acid salts, poly 4-vinylphenol salts, polyvinylsucciniumidum acid salts, sodium-2-sulfoxyethyl methacrylate, sodium-2-acrylamido-2-methylpropane sulphate and sodium-3-acrylamido-3-methyl butanoate; and cationic polymers dimethylaminoethyl methacrylate, dimethylaminoethyl acrylate, diethylaminoethyl methacrylate, diallydimethylammonium chloride, metharylryloxyethyltrimethyl ammonium sulfate, metharylryloxyethyltrimethyl ammoniumchloride, 3-methacrylamidepropyltrimethyl ammonium chloride, polyvinyl pyridine (Blood plasma substitute), quaternerized polyvinylpyridine, polyethyleneimin, linear, polymethylene-N,N-dimethyl piperdinium, polyvinyl 4-alkyl pyridinium, polyvinylbenzenetrimethyl ammonium chloride, 2-acrylamido-2-methylpropane dimethyl ammonium chloride and 1,3 sulfopropyl-2-vinyl pyridinium. The molecular weight of the cationic polymer is preferably between 2 to 80 kDa. The particle size in suspension is preferably less than <200 nm in diameter.

1. Polyethylamine

In certain embodiments, branched chain polycationic polymers are preferred. A particularly preferred branched chain polycationic polymer is the synthetic polycation polyethylenimine (PEI). PEI possesses a high number of amine groups which are arranged in a 1:2:1 ratio of primary:secondary:tertiary amines, which is thought to contribute to its function as a proton sponge and endosome lysis agent.

2. Dendrimer Polycations

In certain embodiments, the polycationic polymer comprises a dendrimer polycation. Dendrimer polycations and methods of preparing them are described in Tomaliaet aL, 1990; PCT/US83/02052; U.S. Pat. Nos. 6,113,946, 4,507,466, 4,558,120, 4,568,737, 4,587,329, 4,631,337, 4,694,064, 4,713,975, 4,737,550, 4,871,779 and 4,857,599, each incorporated herein by reference. Dendrimer polycations generally comprise oligomeric and/or polymeric compounds attached to a core molecule. As used herein “attached” may include, but is not limited to, such attachment means as a covalent bond.

Examples of oligomers and polymers for use in dendrimer polycations include, but are not limited to, polyamidoamines, including but not limited to, methyl acrylate, ethylenediamine or combinations thereof. In certain embodiments the oligomers or polymers are cationic (i.e., capable of being positively charged). In other embodiments, a cationic moiety is attached to the oligomer or polymer. Such cationic moieties include, but are not limited to, guanidinium; azoles, including primary, secondary, tertiary, or quaternary aliphatic or aromatic azoles, and/or S, O, guanidinium or combinations thereof substituted azoles; amides, including primary, secondary, tertiary, or quaternary aliphatic or aromatic amines, and/or S, O, guanidinium or combinations thereof substituted amides; and combinations of guanidinium, azoles and/or amides. The oligomers or polymers may comprise reactive moieties other than cationic moieties. Such reactive moieties include, but are not limited to, hydroxyl, cyano, carboxyl, sulfhydryl, amide, thioether or combinations thereof. The cationic or reactive moieties may comprise or be attached to about 1% to about 100%, and any integer derivable therein, and any range derivable therein, of the oligomer or polymers, or monomers that comprise the oligomers or polymers.

Core molecules include, but are not limited to, ammonia, ethylenediamine, lysine, ornithine, pentaerythritol, tris-(2-aminoethyl)amine or combinations thereof. Core molecules generally comprise at least two reactive moieties that attach the oligomeric and/or polymeric compounds. Such reactive moieties including but not limited to, amino, carboxyhalide maleimide, carboxyl, dethiopyridyl, ester, halide, hydroxyl, imido, imino, sulfhydryl or combinations thereof. Pharmaceutically acceptable core molecules, oligomers and/or polymers are preferred in certain embodiments.

Typical dendrimer polycations are about 2,000 to about 1,000,000 average MW, and any integer derivable therein, and any range derivable therein. Typical dendrimer polycations have a hydrodynamic radius of about 11 to about 60 Å, and any integer derivable therein, and any range derivable therein.

3. Proteinaceous Polycations

In certain embodiments, the polycationic polymer comprises a cationic proteinaceous sequence. Such cationic proteinaceous sequences will preferably comprise one or more cationic amino acid residues or one or more cationic moieties attached to the cationic proteinaceous sequence.

As used herein, the term “cationic proteinaceous sequence” includes, but is not limited to, mixtures of cationic residues, in d and/or l conformation, and/or attached cationic moieties. In certain preferred embodiments, the term “cationic proteinaceous sequence” include amino acid chains comprising one or more arginine, histidine and/or lysine, of either d and/or l isomer conformation. Cationic proteinaceous sequences may also comprise any natural, modified, or unusual amino acid described herein, as long as the majority of residues, i.e., greater than 50%, comprise cationic residues and/or cationic moieties attached to residues of the cationic proteinaceous sequence. A polycationic proteinaceous sequence that comprises more than one different type of amino acid residue is sometimes referred to herein as a “co-polymer.”

Preferred cationic proteinaceous sequences include, but are not limited to poly(1-arginine acid), poly(d-arginine acid), poly(dl-arginine acid), poly(1-histidine acid), poly(d-histidine acid), poly(dl-histidine acid), poly(1-lysine), poly(d-lysine), poly(dl-lysine), copolymers of the above listed polyamino acids with polyethylene glycol, polycaprolactone, polyglycolic acid and polylactic acid, as well as poly(2-hydroxyethyl 1-glutamine), chitosan, carboxymethyl dextran, hyaluronic acid, human serum albumin, and/or alginic acid. In certain embodiments, the cationic proteinaceous sequences of the present invention have a molecular weight of about 1,000, about 2,000, about 3,000, about 4,000, about 5,000, about 6,000, about 7,000, about 8,000, about 9,000, about 10,000, about 11,000, about 12,000, about 13,000, about 14,000, about 15,000, about 16,000, about 17,000, about 18,000, about 19,000, about 20,000, about 21,000, about 22,000, about 23,000, about 24,000, about 25,000, about 26,000, about 27,000, about 28,000, about 29,000, about 30,000, about 31,000, about 32,000, about 33,000, about 34,000, about 35,000, about 36,000, about 37,000, about 38,000, about 39,000, about 40,000, about 41,000, about 42,000, about 43,000, about 44,000, about 45,000, about 46,000, about 47,000, about 48,000, about 49,000, about 50,000, about 51,000, about 52,000, about 53,000, about 54,000, about 55,000, about 56,000, about 57,000, about 58,000, about 59,000, about 60,000, about 61,000, about 62,000, about 63,000, about 64,000, about 65,000, about 66,000, about 67,000, about 68,000, about 69,000, about 70,000, about 71,000, about 72,000, about 73,000, about 74,000, about 75,000, about 76,000, about 77,000, about 78,000, about 79,000, about 80,000, about 81,000, about 82,000, about 83,000, about 84,000, about 85,000, about 86,000, about 87,000, about 88,000, about 89,000, about 90,000, about 91,000, about 92,000, about 93,000, about 94,000, about 95,000, about 96,000, about 97,000, about 98,000, about 99,000, to about 100,000 kd, and any integer derivable therein, and any range derivable therein.

In certain embodiments, various substitutions of naturally occurring, unusual, or chemically modified amino acids may be made in the amino acid composition of the cationic proteinaceous sequences, to obtain molecules having like or otherwise desirable characteristics. For example, a polyamino acid such as poly-arginine, poly-histidine, poly-lysine, or cationic proteinaceous sequences comprising a mixture of arginine, histidine, and/or lysine, may have about 1, about 2, about 3, about 4, about 5, about 6, about 7, about 8, about 9, about 10, about 12, about 13, about 14, about 15, about 16, about 17, about 18, about 19, about 20, about 21, about 22, about 23, about 24, or about 25 or so, and any range derivable therein, of arginine, histidine or lysine, residues, respectively, substituted by any of the naturally occurring, modified, or unusual amino acids described herein. In other aspects of the invention, a cationic proteinaceous sequence such as poly-arginine, poly-histidine, poly-lysine, or a amino acid chain comprising a mixture of some or all of these three amino acids may have about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, to about 50% or so, and any range derivable therein, of the arginine, histidine or lysine residues, respectively, substituted by any of the naturally occurring, modified, or unusual amino acids described herein, as long as the majority of residues comprise histidine, arginine and/or lysine, or attached cationic moieties.

Such substitutions of non-cationic residues and/or moieties to a polyamino acid may provide a convenient chemical moeity for attachment of additional agents, such as, for example, a targeting agent (e.g., a targeting ligand), an endosome agent, a linker/coupling agent, a drug, an anticancer agent or combinations thereof. In a non-limiting example, a glutamic acid residue comprises a side chain carboxyl functional group that can be used to covalently attach agents such as, for example, a drug. Of course, cationic residuce may also serve as points of attachment for one or more additional agents. Such methods of chemical attachment are described herein, and well known to those of ordinary skill in the art (see for example, Li et al., 1996; Greenwald et al., 1996; Van Heeswijk et al., 1985; Hoes et al., 1985; Hirano et al., 1979; Kato et al., 1984; Morimoto et al., 1984; and U.S. Pat. No. 5,362,831, each incorporated herein by reference). In certain aspects the attachment of one or more components may be by a covalent bond directly attaching the agents to the aerosol formulation. In other aspects, the attachment may be by a linker/coupling agent.

Polycationic polymers are relatively easy to deliver and formulate. Polycationic polymers have a self-assembling property when mixed with nucleic acids due to ionic interactions. There have been many studies utilizing the synthetic polycation polyethylenimine (PEI) as a component to deliver nucleic acids to cultured cells as well as to cells in vivo (Bousiff et al., 1995; Boussif et al., 1996).

One or more polycationic polymers are defined as polymers with a plurality of cationic groups. Polycationic polymers include, but are not limited to poly(aminoacids) such as polylysine; polyquaternary compounds; protamine; polyimines; polyvinylamines; polyvinylpyridine; polymethacrylates; polyacrylates; polyoxethanes; polythiodiethyl aminomethylethylene (P(TDAE)); polyhistidine; polyornithine; poly-p-aminostyrene; polyoxethanes; co-polymethacrylates; and polyamidoamines. Polycationic polymers also include the cationic form of gelatins or albumin; cationic phospholipids; and cationic starches. More preferred polycationic polymers of the current invention are protamine and polylysine. The use of polyethyenimine (PEI) is also preferred in this invention.

C. Linkers/Coupling Agents

If desired, the aerosol delivery formulation component(s) of interest may be joined via a biologically-releasable bond, such as a selectively-cleavable linker or amino acid sequence. For example, peptide linkers that include a cleavage site for an enzyme preferentially located or active within a tumor environment are contemplated. Exemplary forms of such peptide linkers are those that are cleaved by urokinase, plasmin, thrombin, Factor IXa, Factor Xa, or a metallaproteinase, such as collagenase, gelatinase, or stromelysin.

In certain embodiments, polyethylene glycol (PEG) is contemplated as a linker/coupling agent. It is contemplated that polyethylene glycol may coat the polycation/nucleic acid combination. It serves as an agent to enhance the fusion of cells-particles polymer particles or lipid particles) and the adhesion of the particles on the pulmonary airway, as well as serves as a point of attachment for additional agents such as targeting ligands. In certain embodiments, for example, the PEG may be attached to the other nucleic acid delivery components by charge (e.g., ionic interactions) and/or covalent bonds.

Additionally, while numerous types of disulfide-bond containing linkers are known which can successfully be employed to conjugate moieties, certain linkers will generally be preferred over other linkers, based on differing pharmacologic characteristics and capabilities. For example, linkers that contain a disulfide bond that is sterically “hindered” are to be preferred, due to their greater stability in vivo, thus preventing release of the moiety prior to binding at the site of action.

Cross-linking reagents are used to form molecular bridges that tie together functional groups of two different molecules, e.g., a stabilizing and coagulating agent. However, it is contemplated that dimers or multimers of the same analog can be made or that heteromeric complexes comprised of different analogs can be created. To link two different compounds in a step-wise manner, hetero-bifunctional cross-linkers can be used that eliminate unwanted homopolymer formation.

TABLE 1
HETERO-BIFUNCTIONAL CROSS-LINKERS

| | | | Spacer Arm |
|---|---|---|---|
| | | Advantages and | Length\after |
| Linker | Reactive Toward | Applications | cross-linking |
| SMPT | Primary amines | Greater stability | 11.2 A |
| | Sulfhydryls | | |
| SPDP | Primary amines | Thiolation | 6.8 A |
| | Sulfhydryls | Cleavable cross-linking | |
| LC-SPDP | Primary amines | Extended spacer arm | 15.6 A |
| | Sulfhydryls | | |
| Sulfo-LC- | Primary amines | Extended spacer arm | 15.6 A |
| SPDP | Sulfhydryls | Water-soluble | |
| SMCC | Primary amines | Stable maleimide | 11.6 A |
| | Sulfhydryls | reactive group | |
| | | Enzyme-antibody | |
| | | conjugation | |
| | | Hapten-carrier | |
| | | protein conjugation | |
| Sulfo-SMCC | Primary amines | Stable maleimide | 11.6 A |
| | Sulfhydryls | reactive group | |
| | | Water-soluble | |
| | | Enzyme-antibody | |
| | | conjugation | |
| MBS | Primary amines | Enzyme-antibody | 9.9 A |
| | Sulfhydryls | conjugation | |
| | | Hapten-carrier | |
| | | protein conjugation | |
| Sulfo-MBS | Primary amines | Water-soluble | 9.9 A |
| | Sulfhydryls | | |
| SIAB | Primary amines | Enzyme-antibody | 10.6 A |
| | Sulfhydryls | conjugation | |
| Sulfo-SIAB | Primary amines | Water-soluble | 10.6 A |
| | Sulfhydryls | | |
| SMPB | Primary amines | Extended spacer arm | 14.5 A |
| | Sulfhydryls | Enzyme-antibody | |
| | | conjugation | |
| Sulfo-SMPB | Primary amines | Extended spacer arm | 14.5 A |
| | Sulfhydryls | Water-soluble | |
| EDC/Sulfo- | Primary amines | Hapten-Carrier | 0 |
| NHS | Carboxyl groups | conjugation | |
| ABH | Carbohydrates | Reacts with sugar groups | 11.9 A |
| | Nonselective | | |

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one proteinaceous compound (e.g., a selected antibody or fragment) and through the thiol reactive group, the cross-linker, already tied up to the first proteinaceous compound, reacts with the cysteine residue (free sulfhydryl group) of the other proteinaceous compound (e.g., another agent).

It is preferred that a cross-linker having reasonable stability in blood will be employed. Numerous types of disulfide-bond containing linkers are known that can be successfully employed to conjugate various agents. Linkers that contain a disulfide bond that is sterically hindered may prove to give greater stability in vivo, preventing release of an agent, such as, for example, a targeting agent, prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1987). The use of such cross-linkers is well understood in the art. Another embodiment involves the use of flexible linkers.

U.S. Pat. No. 4,680,338, describes bifunctional linkers useful for producing conjugates of ligands with amine-containing polymers and/or proteinaceous compounds, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of an agent. Preferred uses include adding a free amino or free sulfhydryl group to a proteinaceous molecule, such as, for example, an antibody or a drug.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

D. Proteinaceous Compositions

In certain embodiments, the present invention concerns a novel aerosol formulation comprising at least one proteinaceous molecule. As used herein, a “proteinaceous molecule,” “proteinaceous composition,” “proteinaceous compound,” “proteinaceous chain,” “proteinaceous sequence” or “proteinaceous material” generally refers, but is not limited to, a protein of greater than about 200 amino acids or the full length endogenous sequence translated from a gene; a polypeptide of greater than about 100 amino acids; and/or a peptide of from about 3 to about 100 amino acids. All the “proteinaceous” terms described above may be used interchangeably herein.

In certain embodiments the size of the at least one proteinaceous molecule may comprise, but is not limited to about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500, about 2750, about 3000, about 3250, about 3500, about 3750, about 4000, about 4250, about 4500, about 4750, about 5000, about 6000, about 7000, about 8000, about 9000, about 10000 or greater amino molecule residues, and any integer derivable therein, and any range derivable therein.

As used herein, an “amino molecule” refers to any amino acid, amino acid derivative or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

Accordingly, the term “proteinaceous composition” encompasses amino molecule sequences comprising at least one of the 20 common amino acids in naturally synthesized proteins, or at least one modified or unusual amino acid, including but not limited to those shown on Table 2 below.

TABLE 2
Modified and Unusual Amino Acids

| Abbr. | Amino Acid | Abbr. | Amino Acid |
|---|---|---|---|
| Aad | 2-Aminoadipic acid | EtAsn | N-Ethylasparagine |
| Baad | 3-Aminoadipic acid | Hyl | Hydroxylysine |
| Bala | β-alanine, β-Amino-propionic acid | Ahyl | allo-Hydroxylysine |
| Abu | 2-Aminobutyric acid | 3Hyp | 3 -Hydroxyproline |
| 4Abu | 4-Aminobutyric acid, piperidinic | 4Hyp | 4-Hydroxyproline |
| | acid | | |
| Acp | 6-Aminocaproic acid | Ide | Isodesmosine |
| Ahe | 2-Aminoheptanoic acid | Aile | allo-Isoleucine |
| Aib | 2-Aminoisobutyric acid | MeGly | N-Methylglycine, |
| | | | sarcosine |
| Baib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

In certain embodiments the proteinaceous composition comprises at least one protein, polypeptide or peptide. In further embodiments the proteinaceous composition comprises a biocompatible protein, polypeptide or peptide. As used herein, the term “biocompatible” refers to a substance which produces no significant untoward effects when applied to, or administered to, a given organism according to the methods and amounts described herein. Such untoward or undesirable effects are those such as significant toxicity or adverse immunological reactions. In preferred embodiments, biocompatible protein, polypeptide or peptide containing compositions will generally be mammalian proteins or peptides or synthetic proteins or peptides each essentially free from toxins, pathogens and harmful immunogens.

Proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. The nucleotide and protein, polypeptide and peptide sequences for various genes have been previously disclosed, and may be found at computerized databases known to those of ordinary skill in the art. One such database is the National Center for Biotechnology Information's Genbank and GenPept databases (http://www.ncbi.nlm.nih.gov/). The coding regions for these known genes may be amplified and/or expressed using the techniques disclosed herein or as would be know to those of ordinary skill in the art. Alternatively, various commercial preparations of proteins, polypeptides and peptides are known to those of skill in the art.

In certain embodiments a proteinaceous compound may be purified. Generally, “purified” will refer to a specific or protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

In certain embodiments, the proteinaceous composition may comprise at least one antibody. It is contemplated that antibodies to specific tissues may bind the tissue(s) and foster tighter adhesion of the glue to the tissues after welding. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

It is contemplated that virtually any protein, polypeptide or peptide containing component may be used in the compositions and methods disclosed herein. However, it is preferred in certain embodiments that the proteinaceous material is biocompatible and/or pharmaceutically acceptable. Proteins and peptides suitable for use in this invention may be autologous proteins or peptides, although the invention is clearly not limited to the use of such autologous proteins. As used herein, the term “autologous protein, polypeptide or peptide” refers to a protein, polypeptide or peptide which is derived or obtained from an organism. The “autologous protein, polypeptide or peptide” may then be used as a component of a composition intended for application to the selected animal or human subject. In certain aspects, the autologous proteins or peptides are prepared, for example from a biological sample from a selected donor.

E. Endosome Agents

In some embodiments, the compositions of the present invention comprise an agent that improves endosomal uptake of the composition and/or reduces endosomal degradation. Such agents include, but are not limited to, an agent that acts as a base or buffer, such as, for example, chloroquine or ammonium chloride, an agent that disrupts endosome membranes, such as, for example, fusogenic peptides, or combinations thereof such agents. Fusogenic peptide include, but are not limited to, those derived from the N-terminus of the IIA influenza virus protein or inactivated adenovirus capsids (U.S. Pat. Nos. 6,083,741 and 5,908,777, each incorporated herein by reference).

In certain embodiments, an endosome agent may comprise all or part of the amino acid sequences of transferrin, asialoorosomucoid, insulin or a combination thereof (U.S. Pat. Nos. 5,792,645 and 5,972,600, incorporated herein by reference).

F. Targeting Agents

In certain embodiments, the aerosol delivery formulations described herein may comprise at least one targeting agent to an organelle, cell, tissue, organ or organism. It is contemplated that any targeting agent described herein or known to one of ordinary skill in the art may be used in the compositions and methods of the present invention, either alone or in combination with other targeting agents. In specific embodiments, the targeting agent may be attached to, for example, a polycation, nucleic acid, and/or other composition component.

Various agents for targeting molecules to specific cells, tissue, organs and organisms are known to those of ordinary skill in the art, and may be used in the methods and compositions of the present invention. In certain embodiments, for example, targeting agents may include, but are not limited to, EGF, transferrin, an anti-prostate specific membrane antigen antibody, endothelial specific peptides and bone specific ligands.

In another non-limiting example, a targeting agent may comprise an antibody, cytokine, growth factor, hormone, lymphokine, receptor protein, such as, for example CD4, CD8 or soluble fragments thereof, a nucleic acid which bind corresponding nucleic acids through base pair complementarity, or a combination thereof (U.S. Pat. No. 6,071,533, incorporated herein by reference). In other embodiments, the targeting ligand may comprise a cellular receptor-targeting ligand, a fusogenic ligand, a nucleus targeting ligand, or a combination thereof (U.S. Pat. No. 5,908,777, incorporated herein by reference). In another non-limiting example, the targeting ligand may comprise an integrin receptor ligand, described in U.S. Pat. No. 6,083,741, incorporated herein by reference.

Still further, an aerosol delivery formulation may be used to deliver a pharmaceutically acceptable composition to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present invention.

Certain receptor-mediated nucleic acid targeting vehicles comprise a cell receptor-specific ligand and a nucleic acid-binding agent. Others comprise a cell receptor-specific ligand to which the nucleic acid to be delivered has been operatively attached. Several ligands have been used for receptor-mediated nucleic acid transfer (Wu and Wu, 1987; Wagner et al., 1990; Perales et al., 1994; Myers, EPO 0273085), which establishes the operability of the technique. Specific delivery in the context of another mammalian cell type has been described (Wu and Wu, 1993; incorporated herein by reference). In certain aspects of the present invention, a ligand will be chosen to correspond to a receptor specifically expressed on the target cell population.

II. Lipids

In the present invention, a lipid formulation is used in the aerosol formulation. A lipid is a substance that is characteristically insoluble in water and extractable with an organic solvent. Lipids include, for example, the substances comprising the fatty droplets that naturally occur in the cytoplasm as well as the class of compounds which are well known to those of skill in the art which contain long-chain aliphatic hydrocarbons and their derivatives, such as fatty acids, alcohols, amines, amino alcohols, and aldehydes. Of course, compounds other than those specifically described herein that are understood by one of skill in the art as lipids are also encompassed by the compositions and methods of the present invention.

A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glucolipids, sulphatides, lipids with ether and ester-linked fatty acids and polymerizable lipids, and combinations thereof.

A. Lipid Types

A fat may comprise a glycerol and a fatty acid. A typical glycerol is a three carbon alcohol. A fatty acid generally is a molecule comprising a carbon chain with an acidic moeity (e.g., carboxylic acid) at an end of the chain. The carbon chain may be of a fatty acid may be of any length, however, it is preferred that the length of the carbon chain be of from about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, to about 30 or more carbon atoms, and any range derivable therein. However, a preferred range is from about 14 to about 24 carbon atoms in the chain portion of the fatty acid, with about 16 to about 18 carbon atoms being particularly preferred in certain embodiments. In certain embodiments the fatty acid carbon chain may comprise an odd number of carbon atoms, however, an even number of carbon atoms in the chain may be preferred in certain embodiments. A fatty acid comprising only single bonds in its carbon chain is called saturated, while a fatty acid comprising at least one double bond in its chain is called unsaturated.

Specific fatty acids include, but are not limited to, linoleic acid, oleic acid, palmitic acid, stearic acid, lauric acid, myristic acid, arachidic acid, palmitoleic acid, arachidonic acid ricinoleic acid, tuberculosteric acid, lactobacillic acid. An acidic group of one or more fatty acids is covalently bonded to one or more hydroxyl groups of a glycerol. Thus, a monoglyceride comprises a glycerol and one fatty acid, a diglyceride comprises a glycerol and two fatty acids, and a triglyceride comprises a glycerol and three fatty acids.

A phospholipid generally comprises either glycerol or an sphingosine moiety, an ionic phosphate group to produce an amphipathic compound, and one or more fatty acids. Types of phospholipids include, for example, phophoglycerides, wherein a phosphate group is linked to the first carbon of glycerol of a diglyceride, and sphingophospholipids (e.g., sphingomyelin), wherein a phosphate group is esterified to a sphingosine amino alcohol. Another example of a sphingophospholipid is a sulfatide, which comprises an ionic sulfate group that makes the molecule amphipathic. A phospholipid may, of course, comprise further chemical groups, such as for example, an alcohol attached to the phosphate group. Examples of such alcohol groups include serine, ethanolamine, choline, glycerol and inositol. Thus, specific phosphoglycerides include a phosphatidyl serine, a phosphatidyl ethanolamine, a phosphatidyl choline, a phosphatidyl glycerol or a phosphotidyl inositol. Other phospholipids include a phosphocholine, a phosphatidic acid or a diacetyl phosphate. In one aspect, a phosphatidylcholine comprises a dioleoyl-phosphatidylcholine (a.k.a. cardiolipin), an egg phosphatidylcholine, a dipalmitoyl phosphalidycholine, a monomyristoyl phosphatidylcholine, a monopalmitoyl phosphatidylcholine, a monostearoyl phosphatidylcholine, a monooleoyl phosphatidylcholine, a dibutroyl phosphatidylcholine, a divaleroyl phosphatidylcholine, a dicaproyl phosphatidylcholine, a diheptanoyl phosphatidylcholine, a dicapryloyl phosphatidylcholine or a distearoyl phosphatidylcholine.

A glycolipid is related to a sphinogophospholipid, but comprises a carbohydrate group rather than a phosphate group attached to a primary hydroxyl group of the sphingosine. A type of glycolipid called a cerebroside comprises one sugar group (e.g., a glucose or galactose) attached to the primary hydroxyl group. Another example of a glycolipid is a ganglioside (e.g., a monosialoganglioside, a GM1), which comprises about 2, about 3, about 4, about 5, about 6, to about 7 or so sugar groups, that may be in a branched chain, attached to the primary hydroxyl group. In other embodiments, the glycolipid is a ceramide (e.g., lactosylceramide).

A steroid is a four-membered ring system derivative of a phenanthrene. Steroids often possess regulatory functions in cells, tissues and organisms, and include, for example, hormones and related compounds in the progestagen (e.g., progesterone), glucocoricoid (e.g., cortisol), mineralocorticoid (e.g., aldosterone), androgen (e.g., testosterone) and estrogen (e.g., estrone) families. Cholesterol is another example of a steroid, and generally serves structural rather than regulatory functions. Vitamin D is another example of a sterol, and is involved in calcium absorption from the intestine.

A terpene is a lipid comprising one or more five-carbon isoprene groups. Terpenes have various biological functions, and include, for example, vitamin A, coenyzme Q and carotenoids (e.g., lycopene and P-carotene).

B. Cationic Lipid Compositions

Preferred lipids of the current invention are cationic lipids, also contemplated are lipid compositions comprising both cationic and neutral lipids. Since the first description of successful in vitro transfection with cationic lipid by Felgner et al. in 1987, there has been substantial progress in the application of synthetic gene delivery systems. One aspects of this progress is the synthesis of new cationic lipids (see Wheeler et al.; 1996, Lee et al., 1996 herein incorporated by reference). Cationic phospholipids may be used for preparing a lipid composition according to the present invention. In a non-limiting example, stearylamine can be used to confer a positive charge on the lipid composition.

Prefered cationic lipids for use in the areosol formulation of the current invention, include, but are not limited to a diacyl-glycero-ethylphosphocholine such as dipalmitoyl-glyceroethylphosphocholine (DPEPC), DSEPC, DMEPC, DLEPC, DOEPC, or palmitoyl-oleoyl-EPC, a diacyl-dimethylammonium propane such as DSDAP, DPDAP, DMDAP, or DODAP, a diacyl-trimethylammonium propane such as DSTAP, DPTAP, DMTAP, or DOTAP, dimethyldioctadecylammonium (DDAB), N-[1-(2,3-ditetradecyloxy)propyl]-N,N-dimethyl-N-hydroxyethylammonium, bromide (DMRIE), N-[1-(2,3,-dioleyloxy)propyl]-N,N-dimethyl-N-hydroxy ethylammonium bromide (DORIE), N-[1-(2,3-dioleyloxy) propyl]-N,N,N-trimethylammonium chloride (DOTMA), DOSPA, 3-beta-[N—(N′,N′-dimethylaminoethane) carbamoly]cholesterol (DC-Chol), 3-beta-[N-(N,N-dicarbobenzoxyspemidine)carbamoyl]cholesterol, or 3-beta-(N-spemine carbamoyl) cholesterol.

C. Making Lipids

Lipids can be obtained from natural sources, commercial sources or chemically synthesized, as would be known to one of ordinary skill in the art, see for example WO90/11092. For example, phospholipids can be from natural sources, such as egg or soybean phosphatidylcholine, brain phosphatidic acid, brain or plant phosphatidylinositol, heart cardiolipin and plant or bacterial phosphatidylethanolamine. In another example, lipids suitable for use according to the present invention can be obtained from commercial sources. In certain embodiments, stock solutions of lipids in chloroform or chloroform/methanol can be stored at about −20° C. Preferably, chloroform is used as the only solvent since it is more readily evaporated than methanol.

D. Lipid Composition Structures

An enzyme or polypeptide, such as CPT I, associated with a lipid may be dispersed in a solution containing a lipid, dissolved with a lipid, emulsified with a lipid, mixed with a lipid, combined with a lipid, covalently bonded to a lipid, contained as a suspension in a lipid or otherwise associated with a lipid. A lipid or lipid/enzyme associated composition of the present invention is not limited to any particular structure. For example, they may also simply be interspersed in a solution, possibly forming aggregates which are not uniform in either size or shape. In another example, they may be present in a bilayer structure, as micelles, or with a “collapsed” structure. In another non-limiting example, a lipofectamine (Gibco BRL)-enzyme or Superfect (Qiagen)-enzyme complex is also contemplated.

In certain embodiments, a lipid composition may comprise about 1%, about 2%, about 3%, about 4% about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, or any range derivable therein, of a particular lipid, lipid type or non-lipid component such as a drug, protein, sugar, nucleic acids or other material disclosed herein or as would be known to one of skill in the art. Thus, it is contemplated that lipid compositions of the present invention may comprise any of the lipids, lipid types or other components in any combination or percentage range.

E. Emulsions

A lipid may be comprised in an emulsion. A lipid emulsion is a substantially permanent heterogeneous liquid mixture of two or more liquids that do not normally dissolve in each other, by mechanical agitation or by small amounts of additional substances known as emulsifiers. Methods for preparing lipid emulsions and adding additional components are well known in the art (e.g., Baker et al., 1990, incorporated herein by reference).

For example, one or more lipids are added to ethanol or chloroform or any other suitable organic solvent and agitated by hand or mechanical techniques. The solvent is then evaporated from the mixture leaving a dried glaze of lipid. The lipids are resuspended in aqueous media, such as phosphate buffered saline, resulting in an emulsion. To achieve a more homogeneous size distribution of the emulsified lipids, the mixture may be sonicated using conventional sonication techniques, further emulsified using microfluidization (using, for example, a Microfluidizer, Newton, Mass.), and/or extruded under high pressure (such as, for example, 600 psi) using an Extruder Device (Lipex Biomembranes, Vancouver, Canada).

F. Micelles

A lipid may be comprised in a micelle, which may further include a protein such as CPT I. A micelles is a cluster or aggregate of lipid compounds, generally in the form of a lipid monolayer, may be prepared using any micelle producing protocol known to those of skill in the art (e.g., Canfield et al, 1990; El-Gorab et al, 1973; Shinoda et al., 1963; and Fendler et al., 1975, each incorporated herein by reference). For example, one or more lipids are typically made into a suspension in an organic solvent, the solvent is evaporated, the lipid is resuspended in an aqueous medium, sonicated and then centrifuged.

G. Liposomes

In particular embodiments, the lipid comprises a liposome. A “liposome” is a generic term encompassing a variety of single and multilamellar lipid vehicles formed by the generation of enclosed lipid bilayers or aggregates. The combination of a protein and liposome may be characterized as a “proteoliposome.” Thus, a CPT I in a liposome may be referred to as “proteoliposomal CPT I.” Liposomes may be characterized as having vesicular structures with a bilayer membrane, generally comprising a phospholipid, and an inner medium that generally comprises an aqueous composition.

A multilamellar liposome has multiple lipid layers separated by aqueous medium. They form spontaneously when lipids comprising phospholipids are suspended in an excess of aqueous solution. The lipid components undergo self-rearrangement before the formation of closed structures and entrap water and dissolved solutes between the lipid bilayers (Ghosh and Bachhawat, 1991). Lipophilic molecules or molecules with lipophilic regions may also dissolve in or associate with the lipid bilayer.

In specific aspects, a lipid and/or CPT I may be, for example, encapsulated in the aqueous interior of a liposome, interspersed within the lipid bilayer of a liposome, attached to a liposome via a linking molecule that is associated with both the liposome and the CPT I, entrapped in a liposome, complexed with a liposome, etc.

H. Making Liposomes

A liposome used according to the present invention can be made by different methods, as would be known to one of ordinary skill in the art.

A liposome can be prepared by mixing lipids in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 min to 2 hours, depending on the desired volume of the liposomes. The composition can be dried further in a desiccator under vacuum. The dried lipids generally are discarded after about 1 week because of a tendency to deteriorate with time.

Dried lipids can be hydrated at approximately 25-50 mM phospholipid in sterile, pyrogen-free water by rotation until all the lipid film is resuspended. The aqueous liposomes can be then separated into aliquots, each placed in a vial, lyophilized and sealed under vacuum.

In other alternative methods, liposomes can be prepared in accordance with other known laboratory procedures (e.g., see Bangham et al., 1965; Gregoriadis, 1979; Deamer and Uster, 1983; Szoka and Papahadjopoulos, 1978, each incorporated herein by reference in relevant part). These methods differ in their respective abilities to entrap aqueous material and their respective aqueous space-to-lipid ratios.

The dried lipids or lyophilized liposomes prepared as described above may be dehydrated and reconstituted in a solution of inhibitory peptide and diluted to an appropriate concentration with an suitable solvent, e.g., DPBS. The mixture is then vigorously shaken in a vortex mixer. Unencapsulated additional materials, such as agents including but not limited to hormones, drugs, nucleic acid constructs and the like, are removed by centrifugation at 29,000× g and the liposomal pellets washed. The washed liposomes are resuspended at an appropriate total phospholipid concentration, e.g., about 50-200 mM. The amount of additional material or active agent encapsulated can be determined in accordance with standard methods. After determination of the amount of additional material or active agent encapsulated in the liposome preparation, the liposomes may be diluted to appropriate concentrations and stored at 4° C. until use. A pharmaceutical composition comprising the liposomes will usually include a sterile, pharmaceutically acceptable carrier or diluent, such as water or saline solution.

The size of a liposome varies depending on the method of synthesis. Liposomes in the present invention can be a variety of sizes. In certain embodiments, the liposomes are small, e.g., less than about 100 nm, about 90 nm, about 80 nm, about 70 nm, about 60 nm, or less than about 50 nm in external diameter. In preparing such liposomes, any protocol described herein, or as would be known to one of ordinary skill in the art may be used. Additional non-limiting examples of preparing liposomes are described in U.S. Pat. Nos. 4,728,578, 4,728,575, 4,737,323, 4,533,254, 4,162,282, 4,310,505, and 4,921,706; International Applications PCT/US85/01161 and PCT/US89/05040; U.K. Patent Application GB 2193095 A; Mayer et al., 1986; Hope et al., 1985; Mayhew et al. 1987; Mayhew et al., 1984; Cheng et al., 1987; and Liposome Technology, 1984, each incorporated herein by reference).

A liposome suspended in an aqueous solution is generally in the shape of a spherical vesicle, having one or more concentric layers of lipid bilayer molecules. Each layer consists of a parallel array of molecules represented by the formula XY, wherein X is a hydrophilic moiety and Y is a hydrophobic moiety. In aqueous suspension, the concentric layers are arranged such that the hydrophilic moieties tend to remain in contact with an aqueous phase and the hydrophobic regions tend to self-associate. For example, when aqueous phases are present both within and without the liposome, the lipid molecules may form a bilayer, known as a lamella, of the arrangement XY-YX. Aggregates of lipids may form when the hydrophilic and hydrophobic parts of more than one lipid molecule become associated with each other. The size and shape of these aggregates will depend upon many different variables, such as the nature of the solvent and the presence of other compounds in the solution.

The production of lipid formulations often is accomplished by sonication or serial extrusion of liposomal mixtures after (I) reverse phase evaporation (II) dehydration-rehydration (III) detergent dialysis and (IV) thin film hydration. In one aspect, a contemplated method for preparing liposomes in certain embodiments is heating sonicating, and sequential extrusion of the lipids through filters or membranes of decreasing pore size, thereby resulting in the formation of small, stable liposome structures. This preparation produces liposomal/polypeptide or liposomes only of appropriate and uniform size, which are structurally stable and produce maximal activity. Such techniques are well known to those of skill in the art (see, for example Martin, 1990). Of course, any other methods of liposome preparation can be used by the skilled artisan to obtain a desired liposome formulation in the present invention.

I. Lipid-Mediated Delivery

Lipid-mediated nucleic acid delivery and expression of foreign DNA in vitro has been very successful (Nicolau and Sene, 1982; Fraley et al., 1979; Nicolau et al., 1987). Wong et al. (1980) demonstrated the feasibility of lipid-mediated delivery and expression of foreign DNA in cultured chick embryo, HeLa and hepatoma cells. Lipid based non-viral formulations provide an alternative to adenoviral gene therapies. Although many cell culture studies have documented lipid based non-viral gene transfer, systemic gene delivery via lipid based formulations has been limited. A major limitation of non-viral lipid based gene delivery is the low delivery efficiency of the cationic lipids that comprise the non-viral delivery vehicle. The in vivo toxicity of liposomes partially explains the discrepancy between in vitro and in vivo gene transfer results. Another factor contributing to this contradictory data is the difference in lipid vehicle stability in the presence and absence of serum proteins. The interaction between lipid vehicles and serum proteins has a dramatic impact on the stability characteristics of lipid vehicles (Yang and Huang, 1997). Cationic lipids attract and bind negatively charged serum proteins. Lipid vehicles associated with serum proteins are either dissolved or taken up by macrophages leading to their removal from circulation. Current in vivo lipid delivery methods use subcutaneous, intradermal, intratumoral, or intracranial injection to avoid the systemic toxicity and stability problems associated with cationic lipids in the circulation. The interaction of lipid vehicles and plasma proteins is responsible for the disparity between the efficiency of in vitro (Felgner et al., 1987) and in vivo gene transfer (Zhu et al., 1993; Philip et al., 1993; Solodin et al., 1995; Liu et al., 1995; Thierry et al., 1995; Tsukamoto et al., 1995; Aksentijevich et al., 1996).

III. Aerosol Delivery Formulation

An aerosol is a two-phase system containing a gas and individual particles, either in solid or liquid form (Swift, D.L., 1985). The aerosol can be used to deliver pharmaceutically acceptable compositions to the trachea, pharynx, bronchi, etc for the treatment and/or prevention of cancers and other pulmonary related diseases. The aerosol delivery can also be used for delivery of pharmaceutically acceptable compositions to the bloodstream through adsorption of the pharmaceutical agent in the lungs. This can be advantageous because of the rapid adsorption into the blood from the extremely large surface area of the lungs compared to the stomach. Also, pharmaceutically active agents that are destroyed in the stomach can be administered by the aerosol delivery formulation of the current invention.

On inhalation, the aerosol passes through the trachea, which branches more than 17 times into successively smaller tubes that constitute the bronchial network, eventually reaching the grapelike clusters of tiny air sacs known as alveoli. Each breath of air is distributed deep into the lung tissue, to the alveolar epithelium, the surface area of which measures ˜100 m² in adults—roughly equivalent to the surface area of a standard singles tennis court. This large area is made up of about half a billion alveoli, from which oxygen passes into the bloodstream via an extensive capillary network. The barrier for delivery of compounds through the lungs is the tightly packed, single-cell-thick layer known as the pulmonary epithelium. In the lungs, the epithelium of the airway is very different from that of the alveolus. Thick, ciliated, mucus-covered cells line the surface of the airway, but the epithelial cell layer thins out as it reaches deeper into the lungs, until reaching the tightly packed alveolar epithelium. Most protein absorption occurs in the alveoli, where the body absorbs peptides and proteins into the bloodstream by a natural process known as transcytosis (http://pubs.acs.org/hotartcl/chemtech/-97/dec/deep.htm).

The formulation of the present invention is introduced into the lungs by an appropriate method. The aerosol may be generated by a medical nebulizer system which is designed to deliver the aerosol through a mouthpiece, facemask, etc. Various nebulizers are known in the art, such as those described in U.S. Pat. No. 4,268,460, U.S. Pat. No. 4,253,468, U.S. Pat. No. 4,046,146, U.S. Pat. No. 4,649,911, U.S. Pat. No. 4,510,929, U.S. Pat. No. 4,627,432, U.S. Pat. No. 6,089,228 and U.S. Pat. No. 6,138,668, each of which is herein incorporated by reference.

The aerosol formulation can be used to encapsulate either small and large drug molecules. The aerosol formulation can also be used for controlled release of encapsulated drugs over a time period ranging from less than an hour, about 1 hour, about 2 hours, about 3 hours, about 4 hours, about 5 hours, about 6 hours, about 7 hours, about 8 hours, about 9 hours, about 10, about 11 hours, hours, about 12 hours, about 13 hours, about 14 hours, about 15 hours, about 16 hours, about 17 hours, about 18 hours, about 1 day, about 2 days, about 3 days, about 4 days, about 5 days, about 6 days, about 7 days, about 8 days, about 9 days, about 10 days, about 12 days, about 13 days, about 14 days, about 15 days, about 16 days, about 17 days, about 18 days, about 1 month, about 2 months, about 3 months, about 4 months, about 5 months, about 6 months, about 7 months, about 8 months, about 9 months, about 10 months, about 11 months, about 1 year, about 2 years, about 3 years, about 4 years, or any range of time therein.

The aerosol formulation is developed such that the formulation is able to protect the pharmaceutical composition from shearing forces associated with aerosol delivery and other extreme conditions associated with aerosol delivery and deposition in the lungs.

It is known in the art that, in targeting drugs to various tissues of the lungs, the size of the particles delivered is varied to obtain an appropriate size range. Larger particles are generally deposited in the airways or nasopharynx while smaller particles are delivered deeper into the lungs. Small particles, with a mean diameter of less than about 10 μm, 7 μm, 5 μm, 2 μm, 1 μm, 0.5 μm, 0.1 μm or 0.05 μm are preferred for delivery into the lungs. Larger particles will not reach the alveoli. For delivery into the airways, larger particles may be preferred, with a mean diameter of less than about 20 μm, 10 μm or 5 μm. The particles can comprise either solids or liquid drops. The particle size in suspension in a nebulizer is preferably between about 0.05 and 3.0 μm.

An important consideration when considering a formulation for delivery into a patient is the toxicity of the formulation. Many aerosol formulations are made to protect the DNA and increase stability, however, this commonly leads to a composition with high toxicity (Bousiff et al., 1995; Boussif et al., 1996).

IV. Combining Polycationic Polymers, Cationic Lipids and Other Components

In certain embodiments, it is contemplated that a desirable composition includes the combination of two or more of: a polycationic polymer, a lipid, a cationic lipid, polyethyleneglycol (PEG), polyethylenimine (PEI), a nucleic acid, and a pharmaceutically acceptable component. Each of the components may be at a different concentration than the others (either higher concentration or lower concentration), desirable compositions may be produced by an adaptation of the methods described herein.

Thus, in certain embodiments of the present invention, a ratio of concentrations of one of a polycationic polymer, a lipid, a cationic lipid, polyethyleneglycol (PEG), polyethylenimine (PEI), a nucleic acid, or a pharmaceutically acceptable component may be combined with any of the other components. This ratio may be about 1:1, about 1.1:1, about 1.2:1, about 1.3:1, about 1.4:1, about 1.5:1, about 1.6:1, about 1.7:1, about 1.8:1, about 1.9:1, about 2.0:1, about 2.1:1, about 2.2:1, about 2.3:1, about 2.4:1, about 2.5:1, about 2.6:1, about 2.7:1, about 2.8:1, about 2.9:1, about 3.0:1, about 3.1:1, about 3.2:1, about 3.3:1, about 3.4:1, about 3.5:1, about 3.6:1, about 3.7:1, about 3.8:1, about 3.9:1, about 4.0:1, about 4.1:1, about 4.2:1, about 4.3:1, about 4.4:1, about 4.5:1, about 4.6:1, about 4.7:1, about 4.8:1, about 4.9:1, about 5.0:1, about 5.1:1, about 5.2:1, about 5.3:1, about 5.4:1, about 5.5:1, about 5.6:1, about 5.7:1, about 5.8:1, about 5.9:1, about 6.0:1, about 6.1:1, about 6.2:1, about 6.3:1, about 6.4:1, about 6.5:1, about 6.6:1, about 6.7:1, about 6.8:1, about 6.9:1, about 7.0:1, about 7.1:1, about 7.2:1, about 7.3:1, about 7.4:1, about 7.5:1, about 7.6:1, about 7.7:1, about 7.8:1, about 7.9:1, about 8.0:1, about 8.1:1, about 8.2:1, about 8.3:1, about 8.4:1, about 8.5:1, about 8.6:1, about 8.7:1, about 8.8:1, about 8.9:1, about 9.0:1, about 9.1:1, about 9.2:1, about 9.3:1, about 9.4:1, about 9.5:1, about 9.6:1, about 9.7:1, about 9.8:1, about 9.9:1, about 10.0:1, about 10.1:1, about 10.2:1, about 10:3:1, about 10.4:1, about 10.5:1, about 10.6:1, about 10.7:1, about 10.8:1, a bout 10.9:1, about 11.0:1, about 11.1:1, about 11.2:1, about 11.3:1, about 11.4:1, about 11.5:1, about 11.6:1, about 11:1, about 11.8:1, about 11.9:1, about 12.0:1, about 12.2:1, about 12.3:1, about 12.4:1, about 12.5:1, about 12.6:1, about 12.7:1, about 12.8:1, about 12.9:1, about 13.0:1, about 13.1:1, about 13.2:1, about 13.3:1, about 13.4:1, about 13.5:1, about 13.6:1, about 13.7:1, about 13.8:1, about 13.9:1, about 14.0:1, about 14.1:1, about 14.2:1, about 14.3:1, about 14.4:1, about 14.5:1, about 14.6:1, about 14.7:1, about 14.8:1, about 14.9:1, about 15.0:1, about 15.1:1, about 15.2:1, about 15.3:1, about 15.4:1, about 15.5:1, about 15.6:1, about 15.7:1, about 15.8:1, about 15.9:1, about 16.0:1, about 16.1:1, about 16.2:1, about 16.3:1, about 16.4:1, about 16.5:1, about 16.6:1, about 16.7:1, about 16.8:1, about 16.9:1, about 17.0:1, about 17.1:1, about 17.2:1, about 17.3:1, about 17.4:1, about 17.5:1, about 17.6:1, about 17.7:1, about 17.8:1, about 17.9:1, about 18.0:1, about 18.1:1, about 18.2:1, about 18.3:1, about 18.4:1, about 18.5:1, about 18.6:1, about 18.7:1, about 18.8:1, about 18.9:1, about 19.0:1, about 19.1:1, about 19.2:1, about 19.3:1, about 19.4:1, about 19.5:1, about 19.6:1, about 19.7:1, about 19.8:1, about 19.9:1, about 20.0:1, about 50:1, about 100:1, about 500:1, about 1,000:1, about 5,000:1, about 10,000:1, about 100,000: 1, about 1,000,000:1 or greater , and any integer derivable therein, and any range derivable therein. In a non-limiting example of such a derivable range, the concentration ratio may be less than about 6.0:1. In a non-limiting example of such a derivable range, the concentration ratio may be less than about 1:6.0. In another non-limiting example of such a derivable range, the concentration ratio may be less than about 1.4:1 to about 6.0:1. In another non-limiting example of such a derivable range, the concentration ratio may be less than about 1.4:1 to about 5.0:1. In another non-limiting example of such a derivable range, the concentration ratio may be less than about 4.0:1. In another non-limiting example of such a derivable range, the concentration ratio may be less than about 1.4:1 to about 3.5:1. In another non-limiting example of such a derivable range, the concentration ration may be less than about 1.4:1 to about 3.0:1. In another non-limiting example of such a derivable range, the concentration ration may be less than about 2:1 to about 3.0:1. In another non-limiting example of such a derivable range, the concentration ratio may be less than about 5.0:1. In another non-limiting example of such a derivable range, the concentration ratio may be less than about 4.0:1. In another non-limiting example of such a derivable range, the concentration ratio may be less than about 3.5:1. In another non-limiting example of such a derivable range, the concentration ratio may be less than about 3.0:1.

Thus, in certain embodiments of the present invention, a ratio of volumes of a liquid composition (e.g., solution, emulsion, suspension, etc.) comprising either one of a polycationic polymer or a nucleic acid combined with another liquid medium comprising the other component may be about 1.4:1, about 1.5:1, about 1.6:1, about 1.7:1, about 1.8:1, about 1.9:1, about 2.0:1, about 2.1:1, about 2.2:1, about 2.3:1, about 2.4:1, about 2 .5:1, about 2.6:1, about 2.7:1, about 2.8:1, about 2.9:1, about 3.0:1, about 3.1:1, about 3.2:1, about 3.3:1, about 3.4:1, about 3.5:1, about 3.6:1, about 3.7:1, about 3.8:1, about 3.9:1, about 4.0:1, about 4.1:1, about 4.2:1, about 4.3:1, about 4.4:1, about 4.5:1, about 4.6:1, about 4.7:1, about 4.8:1, about 4.9:1, about 5.0:1, about 5.1:1, about 5.2:1, about 5.3:1, about 5.4:1, about 5.5:1, about 5.6:1, about 5.7:1, about 5.8:1, about 5.9:1, about 6.0:1, about 6.1:1, about 6.2:1, about 6.3:1, about 6.4:1, about 6.5:1, about 6.6:1, about 6.7:1, about 6.8:1, about 6.9:1, about 7.0:1, about 7.1:1, about 7.2:1, about 7.3:1, about 7.4:1, about 7.5:1, about 7.6:1, about 7.7:1, about 7.8:1, about 7.9:1, about 8.0:1, about 8.1:1, about 8.2:1, about 8.3:1, about 8.4:1, about 8.5:1, about 8.6:1, about 8.7:1, about 8.8:1, about 8.9:1, about 9.0:1, about 9.1:1, about 9.2:1, about 9.3:1, about 9.4:1, about 9.5:1, about 9.6:1, about 9.7:1, about 9.8:1, about 9.9:1, about 10.0:1, about 10.1:1, about 10.2:1, about 10:3:1, about 10.4:1, about 10.5:1, about 10.6:1, about 10.7:1, about 10.8:1, about 10.9:1, about 11.0:1, about 11.1:1, about 11.2:1, about 11.3:1, about 11.4:1, about 11.5:1, about 11.6:1, about 11.:1, about 11.8:1, about 11.9:1, about 12.0:1, about 12.2:1, about 12.3:1, about 12.4:1, about 12.5:1, about 12.6:1, about 12.7:1, about 12.8:1, about 12.9:1, about 13.0:1, about 13.1:1, about 13.2:1, about 13.3:1, about 13.4:1, about 13.5:1, about 13.6:1, about 13.7:1, about 13.8:1, about 13.9:1, about 14.0:1, about 14.1:1, about 14.2:1, about 14.3:1, about 14.4:1, about 14.5:1, about 14.6:1, about 14.7:1, about 14.8:1, about 14.9:1, about 15.0:1, about 15.1:1, about 15.2:1, about 15.3:1, about 15.4:1, about 15.5:1, about 15.6:1, about 15.7:1, about 15.8:1, about 15.9:1, about 16.0:1, about 16.1:1, about 16.2:1, about 16.3:1, about 16.4:1, about 16.5:1, about 16.6:1, about 16.7:1, about 16.8:1, about 16.9:1, about 17.0:1, about 17.1:1, about 17.2:1, about 17.3:1, about 17.4:1, about 17.5:1, about 17.6:1, about 17.7:1, about 17.8:1, about 17.9:1, about 18.0:1, about 18.1:1, about 18.2:1, about 18.3:1, about 18.4:1, about 18.5:1, about 18.6:1, about 18.7:1, about 18.8:1, about 18.9:1, about 19.0:1, about 19.1:1, about 19.2:1, about 19.3:1, about 19.4:1, about 19.5:1, about 19.6:1, about 19.7:1, about 19.8:1, about 19.9:1, about 20.0:1, about 50:1, about 100:1, about 500:1, about 1,000:1, about 5,000:1, about 10,000:1, about 1100,000:1, about 1,000,000:1 or greater, and any integer derivable therein, and any range derivable therein. In a non-limiting example of such a derivable range, the volume of liquid medium comprising polycationic polymer to liquid medium comprising a nucleic acid may be less than about 6.0:1. In a non-limiting example of such a derivable range, the volume of liquid medium comprising polycationic polymer to liquid medium comprising a nucleic acid may be less than about 1:6.0. In another non-limiting example, the volume of liquid medium comprising polycationic polymer to solution comprising a nucleic acid may be between about 1.4:1 to about 6.0:1. In another non-limiting example, the volume of liquid medium comprising polycationic polymer to solution comprising a nucleic acid may be between about 1.4:1 to about 5.0:1. In another non-limiting example, the volume of liquid medium comprising polycationic polymer to solution comprising a nucleic acid may be between about 1.4:1 to about 4.0:1. In another non-limiting example, the volume of liquid medium comprising polycationic polymer to solution comprising a nucleic acid may be between about 1.4:1 to about 3.5:1. In another non-limiting example, the volume of liquid medium comprising polycationic polymer to solution comprising a nucleic acid may be between about 1.4:1 to about 3.0:1. In another non-limiting example, the volume of liquid medium comprising polycationic polymer to solution comprising a nucleic acid may be between about 2:1 to about 3.0:1. In another non-limiting example, the volume of liquid medium comprising polycationic polymer to solution comprising a nucleic acid may be less than about 5.0:1. In another non-limiting example, the volume of liquid medium comprising polycationic polymer to solution comprising a nucleic acid may be less than about 4.0:1. In another non-limiting example, the volume of liquid medium comprising polycationic polymer to solution comprising a nucleic acid may be less than about 3.5:1. In another non-limiting example, the volume of liquid medium comprising polycationic polymer to solution comprising a nucleic acid may be less than about 3.0:1.

In other embodiments of the present invention, a ratio of cationic moieties or residues of the polycation(s) combined with anionic moieties of the nucleic acid(s), or visa verce is about 1:1, about 1.1:, about 1.2:1, about 1.3:1, about 1.4:1, about 1.5:1, about 1.6:1, about 1.7:1, about 1.8:1, about 1.9:1, about 2.0:1, about 2.1:1, about 2.2:1, about 2.3:1, about 2.4:1, about 2.5:1, about 2.6:1, about 2.7:1, about 2.8:1, about 2.9:1, about 3.0:1, about 3.1:1, about 3.2:1, about 3.3:1, about 3.4:1, about 3.5:1, about 3.6:1, about 3.7:1, about 3.8:1, about 3.9:1, about 4.0:1, about 4.1:1, about 4.2:1, about 4.3:1, about 4.4:1, about 4.5:1, about 4.6:1, about 4.7:1, about 4.8:1, about 4.9:1, about 5.0:1, about 5.1:1, about 5.2:1, about 5.3:1, about 5.4:1, about 5.5:1, about 5.6:1, about 5.7:1, about 5.8:1, about 5.9:1, about 6.0:1, about 6.1:1, about 6.2:1, about 6.3:1, about 6.4:1, about 6.5:1, about 6.6:1, about 6.7:1, about 6.8:1, about 6.9:1, about 7.0:1, about 7.1:1, about 7.2:1, about 7.3:1, about 7.4:1, about 7.5:1, about 7.6:1, about 7.7:1, about 7.8:1, about 7.9:1, about 8.0:1, about 8.1:1, about 8.2:1, about 8.3:1, about 8.4:1, about 8.5:1, about 8.6:1, about 8.7:1, about 8.8:1, about 8.9:1, about 9.0:1, about 9.1:1, about 9.2:1, about 9.3:1, about 9.4:1, about 9.5:1, about 9.6:1, about 9.7:1, about 9.8:1, about 9.9:1, about 10.0:1, about 10.1:1, about 10.2:1, about 10:3:1, about 10.4:1, about 10.5:1, about 10.6:1, about 10.7:1, about 10.8:1, about 10.9:1, about 11.0:1, about 11.1:1, about 11.2:1, about 11.3:1, about 11.4:1, about 11.5:1, about 11.6:1, about 11.:1, about 11.8:1, about 11.9:1, about 12.0:1, about 12.2:1, about 12.3:1, about 12.4:1, about 12.5:1, about 12.6:1, about 12.7:1, about 12.8:1, about 12.9:1, about 13.0:1, about 13.1:1, about 13.2:1, about 13.3:1, about 13.4:1, about 13.5:1, about 13.6:1, about 13.7:1, about 13.8:1, about 13.9:1, about 14.0:1, about 14.1:1, about 14.2:1, about 14.3:1, about 14.4:1, about 14.5:1, about 14.6:1, about 14.7:1, about 14.8:1, about 14.9:1, about 15.0:1, about 15.1:1, about 15.2:1, about 15.3:1, about 15.4:1, about 15.5:1, about 15.6:1, about 15.7:1, about 15.8:1, about 15.9:1, about 16.0:1, about 16.1:1, about 16.2:1, about 16.3:1, about 16.4:1, about 16.5:1, about 16.6:1, about 16.7:1, about 16.8:1, about 16.9:1, about 17.0:1, about 17.1:1, about 17.2:1, about 17.3:1, about 17.4:1, about 17.5:1, about 17.6:1, about 17.7:1, about 17.8:1, about 17.9:1, about 18.0:1, about 18.1:1, about 18.2:1, about 18.3:1, about 18.4:1, about 18.5:1, about 18.6:1, about 18.7:1, about 18.8:1, about 18.9:1, about 19.0:1, about 19.1:1, about 19.2:1, about 19.3:1, about 19.4:1, about 19.5:1, about 19.6:1, about 19.7:1, about 19.8:1, about 19.9:1, about 20.0:1, about 50:1, about 100:1, about 500:1, about 1,000:1, about 5,000:1, about 10,000:1, about 100,000:1, about 1,000,000:1 or greater, and any integer derivable therein, and any range derivable therein. In a non-limiting example of a range of cationic moieties to anionic moieties, the number of cationic moieties to anionic moieties may be less than about 6.0:1. In another non-limiting example, the number of cationic moieties to anionic moieties may be less than about 1.4:1 to about 6.0:1. In another non-limiting example, the number of cationic moieties to anionic moieties may be less than about 1.4:1 to about 5.0:1. In another non-limiting example, the number of cationic moieties to anionic moieties may be less than about 1.4:1 to about 4.0:1. In another non-limiting example, the number of cationic moieties to anionic moieties may be less than about 1.4:1 to about 3.5:1. In another non-limiting example, the number of cationic moieties to anionic moieties may be less than about 1.4:1 to about 3.0:1. In another non-limiting example, the number of cationic moieties to anionic moieties may be less than about 2:1 to about 3.0:1. In another non-limiting example, the number of cationic moieties to anionic moieties may be less than about 5.0:1. In another non-limiting example, the number of cationic moieties to anionic moieties may be less than about 4.0:1. In another non-limiting example, the number of cationic moieties to anionic moieties may be less than about 3.5:1. In another non-limiting example, the number of cationic moieties to anionic moieties may be less than about 3.0:1. In a further non-limiting example, the number of cationic to anionic moieties are about 2.4:1 to about 2.7:1. In an additional non-limiting example, the number of cationic moieties to anionic moieties is from about 1.5:1 to about 6:1.

The compositions comprising the polycationic polymers, cationic lipids, PEG, PEI, nucleic acid(s), and pharmaceutically acceptable agents may be combined by any method described herein or as would be known to one of ordinary skill in the art. For example, the composition comprising a polycationic polymer may be added to a composition comprising a nucleic acid, composition comprising a nucleic acid may be added to a composition comprising a polycation, and/or both compositions may be added to each other. Other non-limiting examples of adding various aerosol delivery formulation components are described herein.

V. Nucleic Acid Compositions

Certain embodiments of the present invention concern a purified nucleic acid. In certain aspects, a purified nucleic acid comprises a wild-type or a mutant nucleic acid. In particular aspects, a nucleic acid encodes for or comprises a transcribed nucleic acid. In particular aspects, a nucleic acid encodes a protein, polypeptide, peptide.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 8 and about 100 nucleobases in length. The term “polynucleotide” refers to at least one molecule of greater than about 100 nucleobases in length.

These definitions generally refer to a single-stranded molecule, but in specific embodiments will also encompass an additional strand that is partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule. As used herein, a single stranded nucleic acid may be denoted by the prefix “ss”, a double stranded nucleic acid by the prefix “ds”, and a triple stranded nucleic acid by the prefix “ts.”

A. Nucleobases

As used herein a “nucleobase” refers to a heterocyclic base, such as for example, a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those a purine or pyrimidine substituted by one or more of an alkyl, caboxyalkyl, amino, hydroxyl, halogen (i.e., fluoro, chloro, bromo, or iodo), thiol or alkylthiol moiety. Preferred alkyl (e.g., alkyl, caboxyalkyl, etc.) moieties comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like. A table of non-limiting, purine and pyrimidine derivatives and analogs is also provided herein below.

TABLE 3
Purine and Pyrmidine Derivatives or Analogs

| Abbr. | Modified base description | Abbr. | Modified base description |
|---|---|---|---|
| Ac4c | 4-acetylcytidine | Mam | 5-methoxyaminomethyl-2- |
| | | 5s2u | thiouridine |
| Chm | 5-(carboxyhydroxyl- | Man | Beta,D-mannosylqueosine |
| 5u | methyl)-uridine | q | |
| Cm | 2′-O-methylcytidine | Mcm | 5-methoxycarbonylmethyl-2- |
| | | 5s2u | thiouridine |
| Cmn | 5-carboxymethylamino- | Mcm | 5-methoxycarbonylmethyl- |
| m5s | methyl-2-thioridine | 5u | uridine |
| 2u | | | |
| Cmn | 5-carboxymethylamino- | Mo5u | 5-methoxyuridine |
| m5u | methyluridine | | |
| D | Dihydrouridine | Ms2i | 2-methylthio-N6-isopentenyl- |
| | | 6a | adenosine |
| Fm | 2′-O-methylpseudouridine | Ms2t | N-((9-beta-D-ribofuranosyl-2- |
| | | 6a | methylthiopurine-6- |
| | | | yl)carbamoyl)threonine |
| Gal q | Beta,D-galactosylqueosine | Mt6a | N-((9-beta-D- |
| | | | ribofuranosylpurine-6-yl)N- |
| | | | methyl-carbamoyl)threonine |
| Gm | 2′-O-methylguanosine | Mv | Uridine-5-oxyacetic acid |
| | | | methylester |
| I | Inosine | o5u | Uridine-5-oxyacetic acid (v) |
| I6a | N6-isopentenyladenosine | Osyw | Wybutoxosine |
| m1a | 1-methyladenosine | P | Pseudouridine |
| m1f | 1-methylpseudouridine | Q | Queosine |
| m1g | 1-methylguanosine | s2c | 2-thiocytidine |
| m1I | 1 -methylinosine | s2t | 5-methyl-2-thiouridine |
| m22g | 2,2-dimethylguanosine | s2u | 2-thiouridine |
| m2a | 2-methyladenosine | s4u | 4-thiouridine |
| m2g | 2-methylguanosine | T | 5-methyluridine |
| m3c | 3-methylcytidine | t6a | N-((9-beta-D- |
| | | | ribofuranosylpurine-6- |
| | | | yl)carbamoyl)threonine |
| m5c | 5-methylcytidine | Tm | 2′-O-methyl-5-methyluridine |
| m6a | N6-methyladenosine | Um | 2′-O-methyluridine |
| m7g | 7-methylguanosine | Yw | Wybutosine |
| Mam | 5-methylaminomethyl- | X | 3-(3-amino-3-carboxypropyl)- |
| 5u | uridine | | uridine, (acp3)u |

A nucleobase may be comprised in a nucleoside or nucleotide, using any chemical or natural synthesis method described herein or known to one of ordinary skill in the art.

B. Nucleosides

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9 position of a purine or a 7-deazapurine to the 1′-position of a 5-carbon sugar. In another non-limiting example, a nucleoside comprising a pyrimidine nucleobase (i.e., C, T or U) typically covalently attaches a 1 position of a pyrimidine to a 1′-position of a 5-carbon sugar (Komberg and Baker, 1992).

C. Nucleotides

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

D. Nucleic Acid Analogs

A nucleic acid may comprise, or be composed entirely of, a derivative or analog of a nucleobase, a nucleobase linker moiety and/or backbone moiety that may be present in a naturally occurring nucleic acid. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, incorporated herein by reference).

A non-limiting example of a nucleic acid analog is a “polyether nucleic acid”, described in U.S. Pat. No. 5,908,845, incorporated herein by reference. In a polyether nucleic acid, one or more nucleobases are linked to chiral carbon atoms in a polyether backbone.

Another non-limiting example is a “peptide nucleic acid”, also known as a “PNA”, “peptide-based nucleic acid analog” or “PENAM”, described in U.S. Pat. Nos. 5,786,461, 5891,625, 5,773,571, 5,766,855, 5,736,336, 5,719,262, 5,714,331, 5,539,082, and WO 92/20702, each of which is incorporated herein by reference. Peptide nucleic acids generally have enhanced sequence specificity, binding properties, and resistance to enzymatic degradation in comparison to molecules such as DNA and RNA (Egholm et al., 1993; PCT/EP/01219). A peptide nucleic acid generally comprises one or more nucleotides or nucleosides that comprise a nucleobase moiety, a nucleobase linker moeity that is not a 5-carbon sugar, and/or a backbone moiety that is not a phosphate backbone moiety. Examples of nucleobase linker moieties described for PNAs include aza nitrogen atoms, amido and/or ureido tethers (see for example, U.S. Pat. No. 5,539,082). Examples of backbone moieties described for PNAs include an aminoethylglycine, polyamide, polyethyl, polythioamide, polysulfinamide or polysulfonamide backbone moiety.

In certain embodiments, a nucleic acid analogue such as a peptide nucleic acid may be used to inhibit nucleic acid amplification, such as in PCR, to reduce false positives and discriminate between single base mutants, as described in U.S. Pat. No. 5891,625. Other modifications and uses of nucleic acid analogs are known in the art, and are encompassed by the invention. In a non-limiting example, U.S. Pat. No. 5,786,461 describes PNAs with amino acid side chains attached to the PNA backbone to enhance solubility of the molecule. In another example, the cellular uptake property of PNAs is increased by attachment of a lipophilic group. Examples of this is described in U.S. Pat. Nos. 5,766,855, 5,719,262, 5,714,331 and 5,736,336, which describe PNAs comprising naturally and non-naturally occurring nucleobases and alkylamine side chains that provide improvements in sequence specificity, solubility and/or binding affinity relative to a naturally occurring nucleic acid.

E. Preparation of Nucleic Acids

A nucleic acid may be made by any technique known to one of ordinary skill in the art, such as for example, chemical synthesis or recombinant production. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. A non-limiting example of an enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al., 1989, incorporated herein by reference).

VI. VECTORS

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both incorporated herein by reference).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

A. Promoters and Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al., 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination (as per, for example, the Eukaryotic Promoter Data Base EPDB, http://www.epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Tables 4 lists non-limiting examples of elements/promoters that may be employed, in the context of the present invention, to regulate the expression of a RNA. Table 5 provides non-limiting examples of inducible elements, which are regions of a nucleic acid sequence that can be activated in response to a specific stimulus.

TABLE 4
Promoter and/or Enhancer

| Promoter/Enhancer | References |
|---|---|
| Immunoglobulin | Banerji et al., 1983; Gilles et al., 1983; |
| Heavy Chain | Grosschedl et al., 1985; Atchinson et al., 1986, |
| | 1987; Imler et al, 1987; Weinberger et al., 1984; |
| | Kiledjian et al., 1988; Porton et al.; 1990 |
| Immunoglobulin | Queen et al., 1983; Picard et al., 1984 |
| Light Chain | |
| T-Cell Receptor | Luria et al., 1987; Winoto et al., 1989; |
| | Redondo et al.; 1990 |
| HLA DQ a and/or | Sullivan et al., 1987 |
| DQ β | |
| β-Interferon | Goodbourn et al, 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II | Sherman et al., 1989 |
| HLA-Dra | |
| β-Actin | Kawamoto et al., 1988; Ng et al.; 1989 |
| Muscle Creatine | Jaynes et al., 1988; Horlick et al., 1989; |
| Kinase (MCK) | Johnson et al., 1989 |
| Prealbumin | Costa et al., 1988 |
| (Transthyretin) | |
| Elastase I | Ornitz et al., 1987 |
| Metallothionein | Karin et al., 1987; Culotta et al., 1989 |
| (MTII) | |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| γ-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion | Hirsh et al., 1990 |
| Molecule (NCAM) | |
| α₁-Antitrypain | Latimer et al., 1990 |
| H2B (TH2B) Histone | Hwang et al., 1990 |
| Mouse and/or Type I | Ripe et al., 1989 |
| Collagen | |
| Glucose-Regulated | Chang et al., 1989 |
| Proteins | |
| (GRP94 and GRP78) | |
| Rat Growth Hormone | Larsen et al., 1986 |
| Human Serum | Edbrooke et al., 1989 |
| Amyloid A (SAA) | |
| Troponin I (TN I) | Yutzey et al., 1989 |
| Platelet-Derived | Pech et al., 1989 |
| Growth Factor (PDGF) | |
| Duchenne Muscular | Klamut et al., 1990 |
| Dystrophy | |
| SV40 | Banerji et al., 1981; Moreau et al., 1981; Sleigh et |
| | al., 1985; Firak et al., 1986; Herr et at., 1986; |
| | Imbra et al., 1986; Kadesch et al., 1986; Wang et |
| | al., 1986; Ondek et al., 1987; Kuhl et al., 1987; |
| | Schaffner et al., 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; |
| | Hen et al., 1986; Satake et al., 1988; Campbell |
| | and/or Villarreal, 1988 |
| Retroviruses | Kriegler et al., 1982, 1983; Levinson et al., 1982; |
| | Kriegler et al., 1983, 1984a, b, 1988; Bosze et |
| | al., 1986; Miksicek et al., 1986; Celander et |
| | al., 1987; Thiesen et al., 1988; Celander et |
| | al., 1988; Choi et al., 1988; Reisman et al., 1989 |
| Papilloma Virus | Campo et al., 1983; Lusky et al, 1983; Spandidos |
| | and/or Wilkie, 1983; Spalholz et al., 1985; |
| | Lusky et al., 1986; Cripe et al., 1987; Gloss et |
| | al., 1987; Hirochika et al., 1987; Stephens et |
| | al., 1987 |
| Hepatitis B Virus | Bulla et al., 1986; Jameel et al., 1986; Shaul et |
| | al., 1987; Spandau et al, 1988; Vannice et |
| | al., 1988 |
| Human Immuno- | Muesing et al., 1987; Hauber et al, 1988; |
| deficiency Virus | Jakobovits et al., 1988; Feng et al., 1988; |
| | Takebe et al., 1988; Rosen et al., 1988; |
| | Berkhout et al, 1989; Laspia et al., 1989; Sharp et |
| | al., 1989; Braddock et al., 1989 |
| Cytomegalovirus | Weber et al., 1984; Boshart et al., 1985; |
| (CMV) | Foecking et al., 1986 |
| Gibbon Ape | Holbrook et al, 1987; Quinn et al., 1989 |
| Leukemia Virus | |

TABLE 5
Inducible Elements

| Element | Inducer | References |
|---|---|---|
| MT II | Phorbol Ester | Palmiter et al., 1982; |
| | (TFA) Heavy | Haslinger et al., 1985; Searle |
| | metals | et al., 1985; Stuart et al., |
| | | 1985; Imagawa et al., 1987, |
| | | Karin et al., 1987; Angel et |
| | | al., 1987b; McNeall et |
| | | al., 1989 |
| MMTV (mouse | Glucocorticoids | Huang et al., 1981; Lee et |
| mammary tumor | | at., 1981; Majors et al., 1983; |
| virus) | | Chandler et al., 1983; Lee et |
| | | at., 1984; Ponta et al., 1985; |
| | | Sakai et al., 1988 |
| β-Interferon | Poly(rI)x | Tavernier et al., 1983 |
| | Poly(rc) | |
| Adenovirus 5 E2 | E1A | Imperiale et al., 1984 |
| Collagenase | Phorbol Ester | Angel et al., 1987a |
| | (TPA) | |
| Stromelysin | Phorbol Ester | Angel et al., 1987b |
| | (TPA) | |
| SV40 | Phorbol Ester | Angel et al., 1987b |
| | (TPA) | |
| Murine MX Gene | Interferon, | Hug et al., 1988 |
| | Newcastle | |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene | Interferon | Blanar et al., 1989 |
| H-2κb | | |
| HSP70 | E1A, SV40 Large | Taylor et al., 1989, 1990a, |
| | T Antigen | 1990b |
| Proliferin | Phorbol Ester- | Mordacq et al., 1989 |
| | TPA | |
| Tumor Necrosis Factor | PMA | Hensel et al, 1989 |
| Thyroid Stimulating | Thyroid Hormone | Chatterjee et al., 1989 |
| Hormone α Gene | | |

The identity of tissue-specific promoters or elements, as well as assays to characterize their activity, is well known to those of skill in the art. Nonlimiting examples of such regions include the human LIMK2 gene (Nomoto et al., 1999), the somatostatin receptor 2 gene (Krauset aL, 1998), murine epididymal retinoic acid-binding gene (Lareyreet al., 1999), human CD4 (Zhao-Emonetet al., 1998), mouse alpha2 (XI) collagen (Tsumaki, et al., 1998), D1A dopamine receptor gene (Lee, et al., 1997), insulin-like growth factor II (Wu et al., 1997), and human platelet endothelial cell adhesion molecule-1 (Almendro et al., 1996).

B. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple nucleic acids can be efficiently expressed using a single promoter/enhancer to transcribe a single message (see U.S. Pat. Nos. 5,925,565 and 5,935,819, each herein incorporated by reference).

C. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

D. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference.)

E. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to be more stable and are translated more efficiently. Thus, in other embodiments involving eukaryotes, it is preferred that the terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

F. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

G. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

H. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genetic constructs that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is calorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

I. Plasmid Vectors

In certain embodiments, a plasmid vector is contemplated for use to transform a host cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells, In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEM™-1 1 may be utilized in making a recombinant phage vector which can be used to transform host cells, such as, for example, E. coli LE392.

Further useful plasmid vectors include pIN vectors (Inouye et al., 1985); and pGEX vectors, for use in generating glutathione S-transferase (GST) soluble fusion proteins for later purification and separation or cleavage. Other suitable fusion proteins are those with β-galactosidase, ubiquitin, and the like.

Bacterial host cells, for example, E. coli, comprising the expression vector, are gr own in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

VII. Nucleic Acid Delivery and Cell Transformation

Suitable methods for contacting a nucleic acid component or pharmaceutically acceptable agent with a cell, for transformation of an organelle, a cell, a tissue or an organism, for use with the current invention are believed to include virtually any method by which a nucleic acid (e.g., DNA) can be introduced into an organelle, a cell, a tissue or an organism, as described herein or as would be known to one of ordinary skill in the art. Such methods can be adapted to use with the aerosol formulation of the present invention.

VIII. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous nucleic acid encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells which do not contain a recombinantly introduced nucleic acid.

In certain embodiments, it is contemplated that RNAs or proteinaceous sequences may be co-expressed with other selected RNAs or proteinaceous sequences in the same host cell. Co-expression may be achieved by co-transfecting the host cell with two or more distinct recombinant vectors. Alternatively, a single recombinant vector may be constructed to include multiple distinct coding regions for RNAs, which could then be expressed in host cells transfected with the single vector.

Host cells may be derived from prokaryotes or eukaryotes, depending upon whether the desired result is replication of the vector or expression of part or all of the vector-encoded nucleic acid sequences. Numerous cell lines and cultures are available for use as a host cell, and they can be obtained through the American Type Culture Collection (ATCC), which is an organization that serves as an archive for living cultures and genetic materials (www.atcc.org).

A tissue may comprise a host cell or cells to be transformed or contacted with a nucleic acid and/or and additional agent. The tissue may be part or separated from an organism. In certain embodiments, a tissue may comprise, but is not limited to, adipocytes, alveolar, ameloblasts, axon, basal cells, blood (e.g., lymphocytes), blood vessel, bone, bone marrow, brain, breast, cartilage, cervix, colon, cornea, embryonic, endometrium, endothelial, epithelial, esophagus, facia, fibroblast, follicular, ganglion cells, glial cells, goblet cells, kidney, liver, lung, lymph node, muscle, neuron, ovaries, pancreas, peripheral blood, prostate, skin, small intestine, spleen, stem cells, stomach, testes, anthers, ascite tissue, and all cancers thereof.

IX. Genetic Therapy Agents

Gene therapy now is becoming a viable alternative to various conventional therapies, especially in the area of cancer treatment. Limitations such as long term expression of transgenes and immuno-destruction of target cells through the expression of vector products, which have been said to limit the implementation of genetic therapies, are not concerns in cancer therapies, where destruction of cancer cells is desired.

A tumor cell resistance to agents, such as chemotherapeutic and radiotherapeutic agents, represents a major problem in clinical oncology. It is important in gene transfer therapies, especially those involving treatment of cancer, to kill as many of the cells as quickly as possible. One goal of current cancer research is to find ways to improve the efficacy of one or more anti-cancer agents by combining such an agent with gene therapy. Thus, the use of “combination” therapies may be favored. Such combinations may include gene therapy and radiotherapy or chemotherapy. For example, Rothet al. (1996) have demonstrated that a combination of DNA damaging agents and p53 gene therapy provides increased killing of tumor cells in vivo. In another example, the herpes simplex-thymidine kinase (HS-tK) gene, when delivered to brain tumors by a retroviral vector system, successfully induced susceptibility to the antiviral agent ganciclovir (Culver et al., 1992). In the context of the present invention, it is contemplated that the aerosol delivery system of the current invention could be used for gene therapy.

Yet another type of combination therapy involves the use of multi-gene therapy. In this situation, more than one therapeutic gene would be transferred into a target cell. The genes could be from the same functional group (e.g., both tumor suppressors, both cytokines, etc.) or from different functional groups (e.g., a tumor suppressor and a cytokine). By presenting particular combinations of therapeutic genes to a target cell, it may be possible to augment the overall effect of either or both genes on the physiology of the target cell.

A. Inducers of Cellular Proliferation

In one embodiment of the present invention, it is contemplated that anti-sense mRNA directed to a particular inducer of cellular proliferation is used to prevent expression of the inducer of cellular proliferation. The proteins that induce cellular proliferation further fall into various categories dependent on function. The commonality of all of these proteins is their ability to regulate cellular proliferation. A table listing non-limiting examples of oncogenes that may be targeted by the methods and compositions of the present invention is shown below.

TABLE 6
Oncogenes

| Gene | Source | Human Disease | Function |
|---|---|---|---|
| Growth | | | FGF family |
| Factors¹ | | | member |
| HST/KS | Transfection | | |
| INT-2 | MMTV promoter | | FGF family |
| | Insertion | | member |
| INTI/ | MMTV promoter | | Factor-like |
| WNTI | Insertion | | |
| SIS | Simian sarcoma | | PDGF B |
| | virus | | |

Receptor Tyrosine Kinases^{1,2}

| | | | |
|---|---|---|---|
| ERBB/ | Avian erytbroblasto- | Amplified, | EGF/TGF-α/ |
| HER | sis Virus; | deleted | amphiregulin/ |
| | ALV promoter | Squamous cell | hetacellulin |
| | Insertion; amplified | Cancer; | receptor |
| | Human tumors | glioblastoma | |
| ERBB-2/ | Transfected from rat | Amplified | Regulated by |
| NEU/ | Glioblatoms | breast, | NDF/heregulin |
| HER-2 | | Ovarian, gastric | and EGF-related |
| | | cancers | factors |
| FMS | SM feline sarcoma | | CSF-1 receptor |
| | virus | | |
| KIT | HZ feline sarcoma | | MGF/Steel |
| | virus | | receptor |
| | | | hematopoieis |
| TRK | Transfection from | | NGF (nerve |
| | Human colon cancer | | growth factor) |
| | | | receptor |
| MET | Transfection from | | Scatter factor/ |
| | Human | | HGF receptor |
| | osteosarcoma | | |
| RET | Translocations and | Sporadic thyroid | Orphan receptor |
| | point mutations | cancer; | Tyr kinase |
| | | Familial | |
| | | medullary | |
| | | Thyroid cancer; | |
| | | multiple | |
| | | endocrine | |
| | | neoplasias 2A | |
| | | and 2B | |
| ROS | URII avian sarcoma | | Orphan receptor |
| | Virus | | Tyr kinase |
| PDGF | Translocation | Chronic | TEL (ETS-like |
| receptor | | | transcription |
| | | Myclomonocytic | factor)/PDGF |
| | | Leukemia | receptor gene |
| | | | fusion |
| TGF-/β | | Colon carcinoma | |
| receptor | | Mismatch | |
| | | mutation Target | |

NONRECEPTOR TYROSINE KINASES¹

| | | | |
|---|---|---|---|
| ABI. | Abelson Mul.V | Chronic | Interact with RB, |
| | | myelogenous | RNA |
| | | Leukemia | polymerase, |
| | | translocation | CRK, CBL |
| | | with BCR | |
| FPS/FES | Avian Fujinami | | |
| | SV; GA FeSV | | |
| LCK | Mul.V (murine | | Src family; T cell |
| | leukemia Virus) | | signaling; |
| | promoter Insertion | | interacts CD4/ |
| | | | CD8 T cells |
| SRC | Avian Rous sarcoma | | Membrane- |
| | Virus | | associated Tyr |
| | | | kinase with |
| | | | signaling |
| | | | function; |
| | | | activated by |
| | | | receptor kinases |
| YES | Avian Y73 virus | | Src family; |
| | | | signaling |

SER/THR PROTEIN KINASES¹

| | | |
|---|---|---|
| AKT | AKT8 murine | Regulated by |
| | retrovirus | PI(3)K?; |
| | | regulate 70-kd |
| | | S6 k? |
| MOS | Maloney murine SV | GVBD; |
| | | cystostatic |
| | | factor; MAP |
| | | kinase kinase |
| PIM-1 | Promoter insertion | |
| | Mouse | |
| RAF/MIL | 3611 murine SV; | Signaling in RAS |
| | MH2 avian SV | Pathway |

MISCELLANEOUS CELL SURFACE¹

| | | | |
|---|---|---|---|
| APC | Tumor | Colon cancer | Interacts with |
| | suppressor | | catenins |
| DCC | Tumor | Colon cancer | CAM domains |
| | suppressor | | |
| E-cadherin | Candidate | Breast cancer | Extracellular |
| | tumor | | homotypic |
| | Suppressor | | binding; |
| | | | intracellular |
| | | | interacts with |
| | | | catenins |
| PTC/ | Tumor | Nevoid basal cell | 12 transmem- |
| NBCCS | suppressor and | cancer Syndrome | brane domain; |
| | Drosophilia | (Gorline Syndrome) | signals through |
| | homology | | Gli homogue |
| | | | CI to |
| | | | antagonize |
| | | | hedgehog |
| | | | pathway |
| TAN-1 | Translocation | T-ALI. | Signaling? |
| Notch | | | |
| homolo- | | | |
| gue | | | |

MISCELLANEOUS SIGNALING^{1,3}

| | | | |
|---|---|---|---|
| BCL-2 | Translocation | B-cell lymphoma | Apoptosis |
| CBL | Mu Cas NS-1 | | Tyrosine- |
| | V | | phosphorylated |
| | | | RING |
| | | | finger interact |
| | | | Ab1 |
| CRK | Ct1010 ASV | | Adapted SH2/ |
| | | | SH3 interact |
| | | | Ab1 |
| DPC4 | Tumor | Pancreatic cancer | TGF-β-related |
| | suppressor | | signaling |
| | | | pathway |
| MAS | Transfection | | Possible |
| | and | | angiotensin |
| | Tumorigenicity | | receptor |
| NCK | | | Adaptor SH2/SH3 |

GUANINE NUCLEOTIDE EXCHANGERS AND
BINDING PROTEINS^{3,4}

| | | | |
|---|---|---|---|
| BCR | | Translocated with | Exchanger; |
| | | ABL in CML | protein kinase |
| DBL | Transfection | | Exchanger |
| GSP | | | |
| NF-1 | Hereditary | Tumor suppressor | RAS GAP |
| | tumor | Neurofibromatosis | |
| | Suppressor | | |
| OST | Transfection | | Exchanger |
| Harvey- | HaRat SV; Ki | Point mutations | Signal cascade |
| Kirsten, | RaSV; | in many | |
| N-RAS | Balb- | human tumors | |
| | MoMuSV; | | |
| | Transfection | | |
| VAV | Transfection | | S112/S113; |
| | | | exchanger |

NUCLEAR PROTEINS AND TRANSCRIPTION
FACTORS^{1,5-9}

| | | | |
|---|---|---|---|
| BRCA1 | Heritable | Mammary | Localization |
| | suppressor | cancer/ovarian | unsettled |
| | | cancer | |
| BRCA2 | Heritable | Mammary cancer | Function |
| | suppressor | | unknown |
| ERBA | Avian erythro- | | Thyroid hormone |
| | blastosis | | receptor |
| | Virus | | (transcription) |
| ETS | Avian E26 | | DNA binding |
| | virus | | |
| EVII | MuLV | AML | Transcription |
| | promotor | | factor |
| | Insertion | | |
| FOS | FBI/FBR | | 1 transcription |
| | murine | | factor with |
| | osteosarcoma | | c-JUN |
| | viruses | | |
| GLI | Amplified | Glioma | Zinc finger; |
| | glioma | | cubitus |
| | | | interruptus |
| | | | homologue |
| | | | is in hedgehog |
| | | | signaling |
| | | | pathway; |
| | | | inhibitory link |
| | | | PTC and |
| | | | hedgehog |
| HMGI/ | Translocation | Lipoma | Gene fusions high |
| LIM | t(3:12) | | mobility group |
| | t(12:15) | | HMGI-C |
| | | | (XT-hook) and |
| | | | transcription |
| | | | factor |
| | | | LIM or acidic |
| | | | domain |
| JUN | ASV-17 | | Transcription |
| | | | factor AP-1 with |
| | | | FOS |
| MLL/ | Translocation/ | Acute myeloid | Gene fusion of |
| VHRX + | fusion ELL | leukemia | DNA-binding and |
| ELI/MEN | with MLL | | methyl transferase |
| | Trithorax-like | | MLL with |
| | gene | | ELI RNA pol II |
| | | | elongation factor |
| MYB | Avian | | DNA binding |
| | myeloblastosis | | |
| | Virus | | |
| MYC | Avian MC29; | Burkitt′s lymphoma | DNA binding |
| | Translocation | | with MAX |
| | B-cell | | partner; cyclin |
| | Lymphomas; | | regulation; |
| | promoter | | interact RB?; |
| | Insertion | | regulate |
| | avian leukosis | | apoptosis? |
| | Virus | | |
| N-MYC | Amplified | Neuroblastoma | |
| L-MYC | | Lung cancer | |
| REL | Avian | | NF-κB family |
| | Retriculo- | | transcription |
| | endotheliosis | | factor |
| | Virus | | |
| SKI | Avian SKV770 | | Transcription |
| | Retrovirus | | factor |
| VHL | Heritable | Von Hippel-Landau | Negative |
| | suppressor | Syndrome | regulator or |
| | | | elongin; |
| | | | transcriptional |
| | | | elongation |
| | | | complex |
| WT-¹ | | Wilm's tumor | Transcription |
| | | | factor |

CELL CYCLE/DNA DAMAGE RESPONSE¹⁰⁻²¹

| | | | |
|---|---|---|---|
| ATM | Hereditary | Ataxia- | Protein/lipid |
| | disorder | telangiectasia | kinase homology; |
| | | | DNA damage |
| | | | response |
| | | | upstream in P53 |
| | | | pathway |
| BCL-2 | Bad | Follicular | Anti-poptotic |
| | Bak | lymphoma | Pro-apoptotic |
| | Bax | | Pro-apoptotic |
| | Bid | | Pro-apoptotic |
| | Bik | | Pro-apoptotic |
| | Bim | | Pro-apoptotic |
| | Bok | | Pro-apoptotic |
| FACC | Point mutation | Fanconi's anemia | |
| | | group C | |
| | | (predisposition | |
| | | Leukemia | |
| FHIT | Fragile site | Lung carcinoma | Histidine triad- |
| | 3p14.2 | | related |
| | | | diadenosine 5′, |
| | | | 3″″-P¹.p⁴ |
| | | | tetraphosphate |
| | | | asymmetric |
| | | | hydrolase |
| HML1/ | | HNPCC | Mismatch repair; |
| MutL | | | MutL |
| | | | homologue |
| HMSH2/ | | HNPCC | Mismatch repair; |
| MutS | | | MutS |
| | | | homologue |
| HPMS1 | | HNPCC | Mismatch repair; |
| | | | MutL |
| | | | homologue |
| HPMS2 | | HNPCC | Mismatch repair; |
| | | | MutL |
| | | | homologue |
| INK4/ | Adjacent INK- | Candidate MTS1 | P16 CDK |
| MTSI | 4B at | Suppressor and | inhibitor |
| | 9p21; CDK | MLM Melanoma | |
| | complexes | gene | |
| INK4B/ | | Candidate | P15 CDK |
| MTS2 | | suppressor | inhibitor |
| MDM-2 | Amplified | Sarcoma | Negative |
| | | | regulator p53 |
| p53 | Association | Mutated >50% | Transcription |
| | with SV40 | human tumors, | factor; checkpoint |
| | T antigen | including hereditary | control; apoptosis |
| | | Li-Fraumeni | |
| | | syndrome | |
| PRAD1/ | Translocation | Parathyroid | Cyclin D |
| BCL1 | with | adenoma; B-CLL | |
| | Parathyroid | | |
| | hormone | | |
| | or IgG | | |
| RB | Hereditary | Retinoblastoma; | Interact cyclin/ |
| | Retinoblastoma; | Osteosarcoma; | cdk; regulate E2F |
| | Association | breast cancer; other | transcription |
| | with many | sporadic cancers | factor |
| | DNA virus | | |
| | tumor | | |
| | Antigens | | |
| XPA | | Xeroderma | Excision repair; |
| | | Pigmentosum; skin | photo- |
| | | cancer | product |
| | | predisposition | recognition; |
| | | | zinc finger |

For example, a form of PDGF, the sis oncogene, is a secreted growth factor. Oncogenes rarely arise from genes encoding growth factors, and at the present, is the only known naturally-occurring oncogenic growth factor.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The ErbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

Other proteins such as Jun, Fos and Myc are proteins that directly exert their effects on nuclear functions as transcription factors.

B. Inhibitors of Cellular Proliferation

In certain embodiments, the restoration of the activity of an inhibitor of cellular proliferation through a genetic construct is contemplated. Tumor suppressor oncogenes function to inhibit excessive cellular proliferation. The inactivation of these genes destroys their inhibitory activity, resulting in unregulated proliferation. The tumor suppressors p53, p16 and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as large-T antigen and E1B. The protein is found in normal tissues and cells, but at concentrations which are minute by comparison with transformed cells or tumor tissue.

Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrate, dominant negative alleles (Weinberg, 1991).

Another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16 NK4 has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a newly described class of CDK-inhibitory proteins that also includes p16^{B}, p19, p21^{WAF1}, and p27^{KIP1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Kamb et al., 1994; Okamoto et al., 1994; Nobori et al., 1995; Arap et al., 1995). Restoration of wild-type p16^{INK4} function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf erb, fms, trk, ret, gsp, hst, abl, E1A, p300, genes involved in angiogenesis (e.g., VEGF, FGF, thrombospondin, BAI-1, GDAIF, or their receptors) and MCC.

C. Regulators of Programmed Cell Death

In certain embodiments, it is contemplated that genetic constructs that stimulate apoptosis will be used to promote the death of diseased or undesired tissue. Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., BCl_{XL}, Bcl_{W}, Bcl_{S}, Mcl-1, A1, Bfl-1) or counteract Bcl-2 function and promote cell death (e.g., Bad, Bak, Bax, Bid, Bik, Bim, Bok, Harakiri).

X. Genetic Vaccines

In certain embodiments, an immune response may be promoted by transfecting or inoculating an animal with a nucleic acid encoding an antigen. One or more cells comprised within a target animal then express the sequences encoded by the nucleic acid after administration of the nucleic acid to the animal. Thus, the vaccine may comprise “genetic vaccine” useful for immunization protocols. A vaccine may also be in the form, for example, of a nucleic acid (e.g., a cDNA or an RNA) encoding all or part of the peptide or polypeptide sequence of an antigen. Expression in vivo by the nucleic acid may be, for example, by a plasmid type vector, a viral vector, or a viral/plasmid construct vector.

For a pharmaceutically acceptable formulation to be useful as a vaccine, an antigenic composition encoded by or comprised in a pharmaceutically acceptable formulation must induce an immune response to the antigen in a cell, tissue or animal (e.g., a human). As used herein, an “antigenic composition” may comprise an antigen (e.g., a peptide or polypeptide), a nucleic acid encoding an antigen (e.g., an antigen expression vector), or a cell expressing or presenting an antigen. In other embodiments, the antigenic composition is in a mixture that comprises an additional immunostimulatory agent or nucleic acids encoding such an agent. Immunostimulatory agents include but are not limited to an additional antigen, an immunomodulator, an antigen presenting cell or an adjuvant. In other embodiments, one or more of the additional agent(s) is covalently bonded to the antigen or an immunostimulatory agent, in any combination. In certain embodiments, the antigenic composition is conjugated to or comprises an HLA anchor motif amino acids.

A vaccine of the present invention may vary in its composition of components. In a non-limiting example, a nucleic encoding an antigen might also be formulated with a proteinaceous adjuvant. Of course, it will be understood that various compositions described herein may further comprise additional components. In another non-limiting example, a vaccine may comprise one or more adjuvants. A vaccine of the present invention, and its various components, may be prepared and/or administered by any method disclosed herein or as would be known to one of ordinary skill in the art, in light of the present disclosure.

The nucleotide and protein, polypeptide and peptide encoding sequences for various genes have been previously disclosed, and may be found at computerized databases known to those of ordinary skill in the art. One such database is the National Center for Biotechnology Information's Genbank and GenPept databases (http://www.ncbi.nlm.nih.gov/). The coding regions for these known genes may be amplified, combined (e.g., ligated) with the sequences to produce nucleic acid vectors, described herein, administered to a cell, tissue, organ or organism and/or expressed using the techniques disclosed herein or by any technique that would be know to those of ordinary skill in the art (e.g., Sambrook et al., 1989). Though a nucleic acid may be expressed in an in vitro expression system, in preferred embodiments the nucleic acid comprises a vector for in vivo replication and/or expression.

A. Cellular Vaccine Antigens

In another embodiment, a vaccine may comprise a cell expressing the antigen. The cell may be isolated from a culture, tissue, organ or organism and administered to an animal as a cellular vaccine. Thus, the present invention contemplates a “cellular vaccine.”

The cell may be transfected with a nucleic acid encoding an antigen to enhance its expression of the antigen. Of course, the cell may also express one or more additional vaccine components, such as immunomodulators or adjuvants. A vaccine may comprise all or part of the cell.

In particular embodiments, it is contemplated that nucleic acids encoding antigens of the present invention may be transfected into plants, particularly edible plants, and all or part of the plant material used to prepare a vaccine, such as for example, an oral vaccine. Such methods are described in U.S. Pat. Nos. 5,484,719, 5,612,487, 5,914,123, 5,977,438 and 6,034,298, each incorporated herein by reference.

B. Additional Vaccine Components

It is contemplated that an antigenic composition of the invention may be combined with one or more additional components to form a more effective vaccine. Non-limiting examples of additional components include, for example, one or more additional antigens, immunomodulators or adjuvants to stimulate an immune response to an antigenic composition of the present invention and/or the additional component(s).

1. Immunomodulators

For example, it is contemplated that immunomodulators can be included in the vaccine to augment a cell's or a patient's (e.g. an animal's) response. Immunomodulators can be included as purified proteins, nucleic acids encoding immunomodulators, and/or cells that express immunomodulators in the vaccine composition. The following sections list non-limiting examples of immunomodulators that are of interest, and it is contemplated that various combinations of immunomodulators may be used in certain embodiments (e.g., a cytokine and a chemokine).

Interleukins, cytokines, nucleic acids encoding interleukins or cytokines, and/or cells expressing such compounds are contemplated as possible vaccine components. Interleukins and cytokines, include but are not limited to interleukin 1 (IL-1), IL-2, IL-3, IL-4, IL-5, IL-6, IL-7, IL-8, IL-9, IL-10, IL-11, IL-12, IL-13, IL-14, IL-15, IL-18, β-interferon, α-interferon, γ-interferon, angiostatin, thrombospondin, endostatin, GM-CSF, G-CSF, M-CSF, METH-1, METH-2, tumor necrosis factor, TGFβ, LT and combinations thereof.

Chemokines, nucleic acids that encode for chemokines, and/or cells that express such also may be used as vaccine components. Chemokines generally act as chemo-attractants to recruit immune effector cells to the site of chemokine expression. It may be advantageous to express a particular chemokine coding sequence in combination with, for example, a cytokine coding sequence, to enhance the recruitment of other immune system components to the site of treatment. Such chemokines include, for example, RANTES, MCAF, MIP1-alpha, MIP1-Beta, IP-10 and combinations thereof. The skilled artisan will recognize that certain cytokines are also known to have chemo-attractant effects and could also be classified under the term chemokines.

In certain embodiments, an antigenic composition's may be chemically coupled to a carrier or recombinantly expressed with a immunogenic carrier peptide or polypeptide (e.g., a antigen-carrier fusion peptide or polypeptide) to enhance an immune reaction. Exemplary and preferred immunogenic carrier amino acid sequences include hepatitis B surface antigen, keyhole limpet hemocyanin (KLH) and bovine serum albumin (BSA). Other albumins such as ovalbumin, mouse serum albumin or rabbit serum albumin also can be used as immunogenic carrier proteins. Means for conjugating a polypeptide or peptide to a immunogenic carrier protein are well known in the art and include, for example, glutaraldehyde, m-maleimidobenzoyl-N-hydroxysuccinimide ester, carbodiimide and bis-biazotized benzidine.

It may be desirable to coadminister biologic response modifiers (BRM), which have been shown to upregulate T cell immunity or downregulate suppressor cell activity. Such BRMs include, but are not limited to, cimetidine (CIM; 1200 mg/d) (Smith/Kline, PA); low-dose cyclophosphamide (CYP; 300 mg/m²) (Johnson/Mead, NJ), or a nucleic acid encoding a proteinaceous sequence involved in one or more immune helper functions, such as B-7.

2. Adjuvants

Immunization protocols have used adjuvants to stimulate responses for many years, and as such adjuvants are well known to one of ordinary skill in the art. Some adjuvants affect the way in which antigens are presented. For example, the immune response is increased when protein antigens are precipitated by alum. Emulsification of antigens also prolongs the duration of antigen presentation.

In one aspect, an adjuvant effect is achieved by use of an agent such as alum used in about 0.05 to about 0.1% solution in phosphate buffered saline. Alternatively, the antigen is made as an admixture with synthetic polymers of sugars (Carbopol®) used as an about 0.25% solution. Adjuvant effect may also be made my aggregation of the antigen in the vaccine by heat treatment with temperatures ranging between about 70° to about 101° C. for a 30-second to 2-minute period, respectively. Aggregation by reactivating with pepsin treated (Fab) antibodies to albumin, mixture with bacterial cell(s) such as C. parvum or an endotoxin or a lipopolysaccharide components of Gram minute period, respectively. Aggregation by reactivating with pepsin treated (Fab) antibodies to albumin, mixture with bacterial cell(s) such as C. parvum or an endotoxin or a lipopolysaccharide components of a Gram-active bacteria, emulsion in physiologically acceptable oil vehicles such as mannide mono-oleate (Aracel A) or emulsion with a 20% solution of a perfluorocarbon (Fluosol-DA®) used as a block substitute also may be employed. Some adjuvants, for example, are certain organic molecules obtained from bacteria, act on the host rather than on the antigen. An example is muramyl dipeptide (N-acetylmuramyl-L-alanyl-D-isoglutamine [MDP]), a bacterial peptidoglycan. The effects of MDP, as with most adjuvants, are not fully understood. MDP stimulates macrophages but also appears to stimulate B cells directly. The effects of adjuvants, therefore, are not antigen-specific. If they are administered together with a purified antigen, however, they can be used to selectively promote the response to the antigen.

Adjuvants have been used experimentally to promote a generalized increase in immunity against unknown antigens (e.g., U.S. Pat. No. 4,877,611). This has been attempted particularly in the treatment of cancer. For many cancers, there is compelling evidence that the immune system participates in host defense against the tumor cells, but only a fraction of the likely total number of tumor-specific antigens are believed to have been identified to date. However, using the present invention, the inclusion of a suitable adjuvant into the membrane of an irradiated tumor cell will likely increase the anti-tumor response irrespective of the molecular identification of the prominent antigens. This is a particularly important and time-saving feature of the invention.

In certain embodiments, hemocyanins and hemoerythrins may also be used in the invention. The use of hemocyanin from keyhole limpet (KLH) is preferred in certain embodiments, although other molluscan and arthropod hemocyanins and hemoerythrins may be employed.

Various polysaccharide adjuvants may also be used. For example, the use of various pneumococcal polysaccharide adjuvants on the antibody responses of mice has been described (Yin et al., 1989). The doses that produce optimal responses, or that otherwise do not produce suppression, should be employed as indicated (Yin et al., 1989). Polyamine varieties of polysaccharides are particularly preferred, such as chitin and chitosan, including deacetylated chitin.

Another group of adjuvants are the muramyl dipeptide (MDP, N-acetylmuramyl-L-alanyl-D-isoglutamine) group of bacterial peptidoglycans. Derivatives of muramyl dipeptide, such as the amino acid derivative threonyl-MDP, and the fatty acid derivative MTPPE, are also contemplated.

U.S. Pat. No. 4,950,645 describes a lipophilic disaccharide-tripeptide derivative of muramyl dipeptide which is described for use in artificial liposomes formed from phosphatidyl choline and phosphatidyl glycerol. It is the to be effective in activating human monocytes and destroying tumor cells, but is non-toxic in generally high doses. The compounds of U.S. Pat. No. 4,950,645 and PCT Patent Application WO 91/16347, are contemplated for use with cellular carriers and other embodiments of the present invention.

Another adjuvant contemplated for use in the present invention is BCG. BCG (bacillus Calmette-Guerin, an attenuated strain of Mycobacterium) and BCG-cell wall skeleton (CWS) may also be used as adjuvants in the invention, with or without trehalose dimycolate. Trehalose dimycolate may be used itself. Trehalose dimycolate administration has been shown to correlate with augmented resistance to influenza virus infection in mice (Azuma et al., 1988). Trehalose dimycolate may be prepared as described in U.S. Pat. No. 4,579,945.

BCG is an important clinical tool because of its immunostimulatory properties. BCG acts to stimulate the reticulo-endothelial system, activates natural killer cells and increases proliferation of hematopoietic stem cells. Cell wall extracts of BCG have proven to have excellent immune adjuvant activity. Molecular genetic tools and methods for mycobacteria have provided the means to introduce foreign nucleic acids into BCG (Jacobs et al., 1987; Husson et al., 1990; Martin et al., 1990).

Live BCG is an effective and safe vaccine used worldwide to prevent tuberculosis. BCG and other mycobacteria are highly effective adjuvants, and the immune response to mycobacteria has been studied extensively. With nearly 2 billion immunizations, BCG has a long record of safe use in man (Luelmo, 1982; Lotteet al., 1984). It is one of the few vaccines that can be given at birth, it engenders long-lived immune responses with only a single dose, and there is a worldwide distribution network with experience in BCG vaccination. An exemplary BCG vaccine is sold as TICE® BCG (Organon Inc., West Orange, N.J.).

Amphipathic and surface active agents, e.g., saponin and derivatives such as QS21 (Cambridge Biotech), form yet another group of adjuvants for use with the immunogens of the present invention. Nonionic block copolymer surfactants (Rabinovich et al., 1994; Hunter et al., 1991) may also be employed. Oligonucleotides are another useful group of adjuvants (Yamamoto et al., 1988). Quil A and lentinen are other adjuvants that may be used in certain embodiments of the present invention.

One group of adjuvants preferred for use in the invention are the detoxified endotoxins, such as the refined detoxified endotoxin of U.S. Pat. No. 4,866,034. These refined detoxified endotoxins are effective in producing adjuvant responses in mammals. Of course, the detoxified endotoxins may be combined with other adjuvants to prepare multi-adjuvant-incorporated cells. For example, combination of detoxified endotoxins with trehalose dimycolate is particularly contemplated, as described in U.S. Pat. No. 4,435,386. Combinations of detoxified endotoxins with trehalose dimycolate and endotoxic glycolipids is also contemplated (U.S. Pat. No. 4,505,899), as is combination of detoxified endotoxins with cell wall skeleton (CWS) or CWS and trehalose dimycolate, as described in U.S. Pat. Nos. 4,436,727, 4,436,728 and 4,505,900. Combinations of just CWS and trehalose dimycolate, without detoxified endotoxins, is also envisioned to be useful, as described in U.S. Pat. No. 4,520,019.

In other embodiments, the present invention contemplates that a variety of adjuvants may be employed in the membranes of cells, resulting in an improved immunogenic composition. The only requirement is, generally, that the adjuvant be capable of incorporation into, physical association with, or conjugation to, the cell membrane of the cell in question. Those of skill in the art will know the different kinds of adjuvants that can be conjugated to cellular vaccines in accordance with this invention and these include alkyl lysophosphilipids (ALP); BCG; and biotin (including biotinylated derivatives) among others. Certain adjuvants particularly contemplated for use are the teichoic acids from Gram negative cells. These include the lipoteichoic acids (LTA), ribitol teichoic acids (RTA) and glycerol teichoic acid (GTA). Active forms of their synthetic counterparts may also be employed in connection with the invention (Takada et al., 1995).

Various adjuvants, even those that are not commonly used in humans, may still be employed in animals, where, for example, one desires to raise antibodies or to subsequently obtain activated T cells. The toxicity or other adverse effects that may result from either the adjuvant or the cells, e.g., as may occur using non-irradiated tumor cells, is irrelevant in such circumstances.

One group of adjuvants preferred for use in some embodiments of the present invention are those that can be encoded by a nucleic acid (e.g., DNA or RNA). It is contemplated that such adjuvants may be encoded in a nucleic acid (e.g., an expression vector) encoding the antigen, or in a separate vector or other construct. These nucleic acids encoding the adjuvants can be delivered directly, such as for example with lipids or liposomes.

3. Excipients, Salts and Auxiliary Substances

An antigenic composition of the present invention may be mixed with one or more additional components (e.g., excipients, salts, etc.) which are pharmaceutically acceptable and compatible with at least one active ingredient (e.g., antigen). Suitable excipients are, for example, water, saline, dextrose, glycerol, ethanol and combinations thereof.

An antigenic composition of the present invention may be formulated into the vaccine as a neutral or salt form. A pharmaceutically-acceptable salt, includes the acid addition salts (formed with the free amino groups of the peptide) and those which are formed with inorganic acids such as, for example, hydrochloric or phosphoric acid, or such organic acids as acetic, oxalic, tartaric, mandelic, and the like. A salt formed with a free carboxyl group also may be derived from an inorganic base such as, for example, sodium, potassium, ammonium, calcium, or ferric hydroxide, and such organic bases as isopropylamine, trimethylamine, 2-ethylamino ethanol, histidine, procaine, and combinations thereof In addition, if desired, an antigentic composition may comprise minor amounts of one or more auxiliary substances such as for example wetting or emulsifying agents, pH buffering agents, etc. which enhance the effectiveness of the antigenic composition or vaccine.

D. Vaccine Component Purification

In any case, a vaccine component (e.g., a nucleic acid encoding a proteinaceous composition) may be isolated and/or purified from the chemical synthesis reagents, cell or cellular components. In a method of producing the vaccine component, purification is accomplished by any appropriate technique that is described herein or well known to those of skill in the art (e.g., Sambrook et al., 1989). Although preferred for use in certain embodiments, there is no general requirement that an antigenic composition of the present invention or other vaccine component always be provided in their most purified state. Indeed, it is contemplated that less substantially purified vaccine component, which is nonetheless enriched in the desired compound, relative to the natural state, will have utility in certain embodiments, such as, for example, total recovery of protein product, or in maintaining the activity of an expressed protein. However, it is contemplate that inactive products also have utility in certain embodiments, such as, e.g., in determining antigenicity via antibody generation.

The present invention also provides purified, and in preferred embodiments, substantially purified vaccines or vaccine components. The term “purified vaccine component” as used herein, is intended to refer to at least one vaccine component (e.g., a proteinaceous composition, isolatable from cells), wherein the component is purified to any degree relative to its naturally-obtainable state, e.g., relative to its purity within a cellular extract or reagents of chemical synthesis. In certain aspects wherein the vaccine component is a proteinaceous composition, a purified vaccine component also refers to a wild-type or mutant protein, polypeptide, or peptide free from the environment in which it naturally occurs.

Where the term “substantially purified” is used, this will refer to a composition in which the specific compound (e.g., a protein, polypeptide, or peptide) forms the major component of the composition, such as constituting about 50% of the compounds in the composition or more. In preferred embodiments, a substantially purified vaccine component will constitute more than about 60%, about 70%, about 80%, about 90%, about 95%, about 99% or even more of the compounds in the composition.

In certain embodiments, a vaccine component may be purified to homogeneity. As applied to the present invention, “purified to homogeneity,” means that the vaccine component has a level of purity where the compound is substantially free from other chemicals, biomolecules or cells. For example, a purified peptide, polypeptide or protein will often be sufficiently free of other protein components so that degradative sequencing may be performed successfully. Various methods for quantifying the degree of purification of a vaccine component will be known to those of skill in the art in light of the present disclosure. These include, for example, determining the specific protein activity of a fraction (e.g., antigenicity), or assessing the number of polypeptides within a fraction by gel electrophoresis.

Various techniques suitable for use in chemical, biomolecule or biological purification, well known to those of skill in the art, may be applicable to preparation of a vaccine component of the present invention. These include, for example, precipitation with ammonium sulfate, PEG, antibodies and the like or by heat denaturation, followed by centrifugation; fractionation, chromatographic procedures, including but not limited to, partition chromatograph (e.g., paper chromatograph, thin-layer chromatograph (TLC), gas-liquid chromatography and gel chromatography) gas chromatography, high performance liquid chromatography, affinity chromatography, supercritical flow chromatography ion exchange, gel filtration, reverse phase, hydroxylapatite, lectin affinity; isoelectric focusing and gel electrophoresis (see for example, Sambrook et al., 1989; and Freifelder, Physical Biochemistry, Second Edition, pages 238-246, incorporated herein by reference).

Given many DNA and proteins are known (see for example, the National Center for Biotechnology Information's Genbank and GenPept databases (http://www.ncbi.nlm.nih.gov/)), or may be identified and amplified using the methods described herein, any purification method for recombinately expressed nucleic acid or proteinaceous sequences known to those of skill in the art can now be employed. In certain aspects, a nucleic acid may be purified on polyacrylamide gels, and/or cesium chloride centrifugation gradients, or by any other means known to one of ordinary skill in the art (see for example, Sambrook et al., 1989, incorporated herein by reference). In further aspects, a purification of a proteinaceous sequence may be conducted by recombinantly expressing the sequence as a fusion protein. Such purification methods are routine in the art. This is exemplified by the generation of an specific protein-glutathione S-transferase fusion protein, expression in E. coli, and isolation to homogeneity using affinity chromatography on glutathione-agarose or the generation of a polyhistidine tag on the N- or C-terminus of the protein, and subsequent purification using Ni-affinity chromatography. In particular aspects, cells or other components of the vaccine may be purified by flow cytometry. Flow cytometry involves the separation of cells or other particles in a liquid sample, and is well known in the art (see, for example, U.S. Pat. Nos. 3,826,364, 4,284,412, 4,989,977, 4,498,766, 5,478,722, 4,857,451, 4,774,189, 4,767,206, 4,714,682, 5,160,974 and 4,661,913). Any of these techniques described herein, and combinations of these and any other techniques known to skilled artisans, may be used to purify and/or assay the purity of the various chemicals, proteinaceous compounds, nucleic acids, cellular materials and/or cells that may comprise a vaccine of the present invention. As is generally known in the art, it is believed that the order of conducting the various purification steps may be changed, or that certain steps may be omitted, and still result in a suitable method for the preparation of a substantially purified antigen or other vaccine component.

E. Vaccine Preparations

Once produced, synthesized and/or purified, an antigen or other vaccine component may be prepared as a vaccine for administration to a patient. The preparation of a vaccine is generally well understood in the art, as exemplified by U.S. Pat. Nos. 4,608,251, 4,601,903, 4,599,231, 4,599,230, and 4,596,792, all incorporated herein by reference. Such methods may be used to prepare a vaccine comprising an antigenic composition as active ingredient(s), in light of the present disclosure. In preferred embodiments, the compositions of the present invention are prepared to include pharmacologically acceptable vaccines.

F. Vaccine Administration

A vaccination schedule and dosages may be varied on a patient by patient basis, taking into account, for example, factors such as the weight and age of the patient, the type of disease being treated, the severity of the disease condition, previous or concurrent therapeutic interventions, the manner of administration and the like, which can be readily determined by one of ordinary skill in the art.

A vaccine is administered in a manner compatible with the dosage formulation, and in such amount as will be therapeutically effective and immunogenic. For example, the intramuscular route may be preferred in the case of toxins with short half lives in vivo. The quantity to be administered depends on the subject to be treated, including, e.g., the capacity of the individual's immune system to synthesize antibodies, and the degree of protection desired. The dosage of the vaccine will depend on the route of administration and will vary according to the size of the host. Precise amounts of an active ingredient required to be administered depend on the judgment of the practitioner. In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. However, a suitable dosage range may be, for example, of the order of several hundred micrograms active ingredient per vaccination. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per vaccination, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above. A suitable regime for initial administration and booster administrations (e.g., inoculations) are also variable, but are typified by an initial administration followed by subsequent inoculation(s) or other administration(s). It is also understood that the dosage may be increased for aerosol delivery because of the lower efficiency of delivery compared to intravenous and oral delivery methods.

In many instances, it will be desirable to have multiple administrations of the vaccine, usually not exceeding six vaccinations, more usually not exceeding four vaccinations and preferably one or more, usually at least about three vaccinations. The vaccinations will normally be at from two to twelve week intervals, more usually from three to five week intervals. Periodic boosters at intervals of 1-5 years, usually three years, will be desirable to maintain protective levels of the antibodies.

The course of the immunization may be followed by assays for antibodies for the supernatant antigens. The assays may be performed by labeling with conventional labels, such as radionuclides, enzymes, fluorescent probes, and the like. These techniques are well known and may be found in a wide variety of patents, such as U.S. Pat. Nos. 3,791,932; 4,174,384 and 3,949,064, as illustrative of these types of assays. Other immune assays can be performed and assays of protection from challenge with the antigen can be performed, following immunization.

G. Enhancement of an Immune Response

The present invention includes a method of enhancing the immune response in a subject comprising the steps of contacting one or more lymphocytes with an antigenic composition. In certain embodiments the one or more lymphocytes is comprised in an animal, such as a human. In other embodiments, the lymphocyte(s) may be isolated from an animal or from a tissue (e.g., blood) of the animal. In certain preferred embodiments, the lymphocyte(s) are peripheral blood lymphocyte(s). In certain embodiments, the one or more lymphocytes comprise a T-lymphocyte or a B-lymphocyte. In a particularly preferred facet, the T-lymphocyte is a cytotoxic T-lymphocyte.

The enhanced immune response may be an active or a passive immune response. Alternatively, the response may be part of an adoptive immunotherapy approach in which lymphocyte(s) are obtained with from an animal (e.g., a patient), then pulsed with composition comprising an antigenic composition. In a preferred embodiment, the lymphocyte(s) may be administered to the same or different animal (e.g., same or different donors).

1. Cytotoxic T Lymphocytes

In certain embodiments, T-lymphocytes are specifically activated by contact with an antigenic composition of the present invention. In certain embodiments, T-lymphocytes are activated by contact with an antigen presenting cell that is or has been in contact with an antigenic composition of the invention.

T cells express a unique antigen binding receptor on their membrane (T-cell receptor), which can only recognize antigen in association with major histocompatibility complex (MHC) molecules on the surface of other cells. There are several populations of T cells, such as T helper cells and T cytotoxic cells. T helper cells and T cytotoxic cells are primarily distinguished by their display of the membrane bound glycoproteins CD4 and CD8, respectively. T helper cells secret various lymphokines, that are crucial for the activation of B cells, T cytotoxic cells, macrophages and other cells of the immune system. In contrast, a T cytotoxic cells that recognizes an antigen-MHC complex proliferates and differentiates into an effector cell called a cytotoxic T lymphocyte (CTL). CTLs eliminate cells of the body displaying antigen by producing substances that result in cell lysis.

CTL activity can be assessed by methods described herein or as would be known to one of skill in the art. For example, CTLs may be assessed in freshly isolated peripheral blood mononuclear cells (PBMC), in a phytohaemaglutinin-stimulated IL-2 expanded cell line established from PBMC (Bernard et al., 1998) or by T cells isolated from a previously immunized subject and restimulated for 6 days with DC infected with an adenovirus vector containing antigen using standard 4 h ⁵¹Cr release microtoxicity assays. In another fluorometric assay developed for detecting cell-mediated cytotoxicity, the fluorophore used is the non-toxic molecule alamarBlue (Nociari et al., 1998). The alamarBlue is fluorescently quenched (i.e., low quantum yield) until mitochondrial reduction occurs, which then results in a dramatic increase in the alamarBlue fluorescence intensity (i.e., increase in the quantum yield). This assay is reported to be extremely sensitive, specific and requires a significantly lower number of effector cells than the standard ⁵¹Cr release assay.

In certain aspects, T helper cell responses can be measured by in vitro or in vivo assay with peptides, polypeptides or proteins. In vitro assays include measurement of a specific cytokine release by enzyme, radioisotope, chromaphore or fluorescent assays. In vivo assays include delayed type hypersensitivity responses called skin tests, as would be known to one of ordinary skill in the art.

2. Antigen Presenting Cells

In general, the term “antigen presenting cell” can be any cell that accomplishes the goal of the invention by aiding the enhancement of an immune response (i.e., from the T-cell or -B-cell arms of the immune system) against an antigen. Such cells can be defined by those of skill in the art, using methods disclosed herein and in the art. As is understood by one of ordinary skill in the art (see for example Kuby, 1993, incorporated herein by reference), and used herein certain embodiments, a cell that displays or presents an antigen normally or preferentially with a class II major histocompatability molecule or complex to an immune cell is an “antigen presenting cell.” In certain aspects, a cell (e.g., an APC cell) may be fused with another cell, such as a recombinant cell or a tumor cell that expresses the desired antigen. Methods for preparing a fusion of two or more cells is well known in the art, such as for example, the methods disclosed in Goding, pp. 65-66, 71-74 1986; Campbell, pp. 75-83, 1984; Kohler and Milstein, 1975; Kohler and Milstein, 1976, Gefter et al., 1977, each incorporated herein by reference. In some cases, the immune cell to which an antigen presenting cell displays or presents an antigen to is a CD4⁺ TH cell. Additional molecules expressed on the APC or other immune cells may aid or improve the enhancement of an immune response. Secreted or soluble molecules, such as for example, immunomodulators and adjuvants, may also aid or enhance the immune response against an antigen. Such molecules are well known to one of skill in the art, and various examples are described herein.

XI. Cancer Treatments

A therapeutic composition may be delivered to a cell, tissue or organism for the treatment of cancer using the formulation of the present invention. One or more agents effective in the treatment of hyperproliferative disease, such as, for example, an anti-cancer agent may be used. An “anti-cancer” agent is capable of negatively affecting cancer in a subject, for example, by killing one or more cancer cells, inducing apoptosis in one or more cancer cells, reducing the growth rate of one or more cancer cells, reducing the incidence or number of metastases, reducing a tumor's size, inhibiting a tumor's growth, reducing the blood supply to a tumor or one or more cancer cells, promoting an immune response against one or more cancer cells or a tumor, preventing or inhibiting the progression of a cancer, or increasing the life-span of a subject with a cancer. Anti-cancer agents include, for example, chemotherapy agents (chemotherapy), radiotherapy agents (radiotherapy), a surgical procedure (surgery), immune therapy agents (immunotherapy), genetic therapy agents (gene therapy), hormonal therapy, other biological agents (biotherapy) and/or alternative therapies. Such an agent would be provided either alone or in a combined amount with another agent in an amount effective to kill or inhibit proliferation of a cancer cell.

Cancers that can be treated by the current invention include, but are not limited to cancer of the lung, upper airway primary or secondary, head or neck, bladder, kidneys, pancreas, mouth, throat, pharynx, larynx, esophagus, brain, liver, spleen, kidney, lymph node, small intestine, pancreas, blood cells, colon, stomach, breast, endometrium, prostate, testicle, ovary, skin, bone marrow and blood cancer. It is preferred that lung and upper airway cancers are treated by the aerosol formulation of this invention. These lung and upper airway cancers are defined by a number of histologic classifications including: squamous cell carcinomas such as squamous carcinoma; small cell carcinomas such as oat cell carcinoma, intermediate cell type carcinoma, combined oat and cell carcinoma; adenocarcinomas such as acinar adenocarcinoma, papillary adenocarcinoma, bronchioloalveolar carcinoma, and solid carcinoma with mucus formation; large cell carcinoma such as giant cell carcinoma and clear cell carcinoma; adenosquamous carcinoma; carcinoid; and bronchial gland carcinomas such as adenoid cystic, and mucoepidermoid carcinoma.

Administration of the anti-cancer agent or agents to a cell, tissue or organism may follow general protocols for the administration of chemotherapeutics via aerosol, taking into account the toxicity, if any. It is expected that the treatment cycles would be repeated as necessary. In particular embodiments, it is contemplated that various additional agents may be applied in any combination with the present invention.

A. Chemotherapeutic Agents

The term “chemotherapy” refers to the use of drugs to treat cancer. A “chemotherapeutic agent” is used to connote a compound or composition that is administered in the treatment of cancer. One subtype of chemotherapy known as biochemotherapy involves the combination of a chemotherapy with a biological therapy.

Chemotherapeutic agents include, but are not limited to, 5-fluorouracil, bleomycin, busulfan, camptothecin, carboplatin, chlorambucil, cisplatin (CDDP), cyclophosphamide, dactinomycin, daunorubicin, doxorubicin, epirubicin, estrogen receptor binding agents, etoposide (VP16), famesyl-protein transferase inhibitors, gemcitabine, ifosfamide, mechlorethamine, melphalan, mitomycin, navelbine, nitrosurea, plicomycin, procarbazine, raloxifene, tamoxifen, taxol, temazolomide (an aqueous form of DTIC), transplatinum, topotecan, vinblastine and methotrexate, vincristine, or any analog or derivative variant of the foregoing. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, corticosteroid hormones, mitotic inhibitors, and nitrosoureas, hormone agents, miscellaneous agents, and any analog or derivative variant thereof.

Chemotherapeutic agents and methods of administration, dosages, etc. are well known to those of skill in the art (see for example, the “Physicians Desk Reference”, Goodman & Gilman's “The Pharmacological Basis of Therapeutics” and in “Remington's Pharmaceutical Sciences”, incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject. Examples of specific chemotherapeutic agents and dose regimes are also described herein. Of course, all of these dosages and agents described herein are exemplary rather than limiting, and other doses or agents may be used by a skilled artisan for a specific patient or application. Any dosage in-between these points, or range derivable therein is also expected to be of use in the invention.

Alkylating agents are drugs that directly interact with genomic DNA to prevent the cancer cell from proliferating. This category of chemotherapeutic drugs represents agents that affect all phases of the cell cycle, that is, they are not phase-specific. Alkylating agents can be implemented to treat, for example, chronic leukemia, non-Hodgkin's lymphoma, Hodgkin's disease, multiple myeloma, and particular cancers of the breast, lung, and ovary. An alkylating agent, may include, but is not limited to, a nitrogen mustard, an ethylenimene, a methylmelamine, an alkyl sulfonate, a nitrosourea or a triazines.

They include but are not limited to: busulfan, chlorambucil, cisplatin, cyclophosphamide (cytoxan), dacarbazine, ifosfamide, mechlorethamine (mustargen), and melphalan. In specific aspects, troglitazaone can be used to treat cancer in combination with any one or more of these alkylating agents, some of which are discussed below.

1. Nitrogen Mustards

A nitrogen mustard may be, but is not limited to, mechlorethamine (HN₂), which is used for Hodgkin's disease and non-Hodgkin's lymphomas; cyclophosphamide and/or ifosfamide, which are used in treating such cancers as acute or chronic lymphocytic leukemias, Hodgkin's disease, non-Hodgkin's lymphomas, multiple myeloma, neuroblastoma, breast, ovary, lung, Wilm's tumor, cervix testis and soft tissue sarcomas; melphalan (L-sarcolysin), which has been used to treat such cancers as multiple myeloma, breast and ovary; and chlorambucil, which has been used to treat diseases such as, for example, chronic lymphatic (lymphocytic) leukemia, malignant lymphomas including lymphosarcoma, giant follicular lymphoma, Hodgkin's disease and non-Hodgkin's lymphomas.

Chlorambucil (also known as leukeran) is a bifunctional alkylating agent of the nitrogen mustard type that has been found active against selected human neoplastic diseases. Chlorambucil is known chemically as 4-[bis(2-chlorethyl)amino]benzenebutanoic acid.

Chlorambucil is available in tablet form for oral administration. It is rapidly and completely absorbed from the gastrointestinal tract. For example, after a single oral dose of about 0.6 mg/kg to about 1.2 mg/kg, peak plasma chlorambucil levels are reached within one hour and the terminal half-life of the parent drug is estimated at about 1.5 hours. About 0.1 mg/kg/day to about 0.2 mg/kg/day or about 3 6 mg/m²/day to about 6 mg/m²/day or alternatively about 0.4 mg/kg may be used for antineoplastic treatment. Chlorambucil is not curative by itself but may produce clinically useful palliation.

Cyclophosphamide is 2H-1,3,2-Oxazaphosphorin-2-amine, NN-bis(2-chloroethyl)tetrahydro-, 2-oxide, monohydrate; termed Cytoxan available from Mead Johnson; and Neosar available from Adria. Cyclophosphamide is prepared by condensing 3-amino-1-propanol with NN-bis(2-chlorethyl) phosphoramidic dichloride [(ClCH₂CH₂)₂N-POCl₂] in dioxane solution under the catalytic influence of triethylamine. The condensation is double, involving both the hydroxyl and the amino groups, thus effecting the cyclization.

Unlike other β-chloroethylamino alkylators, it does not cyclize readily to the active ethyleneimonium form until activated by hepatic enzymes. Thus, the substance is stable in the gastrointestinal tract, tolerated well and effective by the oral and parental routes and does not cause local vesication, necrosis, phlebitis or even pain.

Suitable oral doses for adults include, for example, about 1 mg/kg/day to about 5 mg/kg/day (usually in combination), depending upon gastrointestinal tolerance; or about 1 mg/kg/day to about 2 mg/kg/day; intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. In some aspects, a dose of about 250 mg/kg/day may be administered as an antineoplastic. Because of gastrointestinal adverse effects, the intravenous route is preferred for loading. During maintenance, a leukocyte count of about 3000/mm3 to 4000/mm³ usually is desired. The drug also sometimes is administered intramuscularly, by infiltration or into body cavities. It is available in dosage forms for injection of about 100 mg, about 200 mg and about 500 mg, and tablets of about 25 mg and about 50 mg.

Melphalan, also known as alkeran, L-phenylalanine mustard, phenylalanine mustard, L-PAM, or L-sarcolysin, is a phenylalanine derivative of nitrogen mustard. Melphalan is a bifunctional alkylating agent which is active against selective human neoplastic diseases. It is known chemically as 4-[bis(2-chloroethyl)amino]-L-phenylalanine.

Melphalan is the active L-isomer of the compound and was first synthesized in 1953 by Bergel and Stock; the D-isomer, known as medphalan, is less active against certain animal tumors, and the dose needed to produce effects on chromosomes is larger than that required with the L-isomer. The racemic (DL—) form is known as merphalan or sarcolysin. Melphalan is insoluble in water and has a pKa₁ of about 2.1. Melphalan is available in tablet form for oral administration and has been used to treat multiple myeloma. Available evidence suggests that about one third to one half of the patients with multiple myeloma show a favorable response to oral administration of the drug.

Melphalan has been used in the treatment of epithelial ovarian carcinoma. One commonly employed regimen for the treatment of ovarian carcinoma has been to administer melphalan at a dose of about 0.2 mg/kg daily for five days as a single course. Courses are repeated about every four to five weeks depending upon hematologic tolerance (Smith and Rutledge, 1975; Young et al., 1978). Alternatively in certain embodiments, the dose of melphalan used could be as low as about 0.05 mg/kg/day or as high as about 3 mg/kg/day or greater.

2. Ethylenimenes and Methylmelamines

An ethylenimene and/or a methylmelamine include, but are not limited to, hexamethylmelamine, used to treat ovary cancer; and thiotepa, which has been used to treat bladder, breast and ovary cancer.

3. Alkyl Sulfonates

An alkyl sulfonate includes but is not limited to such drugs as busulfan, which has been used to treat chronic granulocytic leukemia.

Busulfan (also known as myleran) is a bifunctional alkylating agent. Busulfan is known chemically as 1,4-butanediol dimethanesulfonate. Busulfan is available in tablet form for oral administration, wherein for example, each scored tablet contains about 2 mg busulfan and the inactive ingredients magnesium stearate and sodium chloride.

Busulfan is indicated for the palliative treatment of chronic myelogenous (myeloid, myelocytic, granulocytic) leukemia. Although not curative, busulfan reduces the total granulocyte mass, relieves symptoms of the disease, and improves the clinical state of the patient. Approximately 90% of adults with previously untreated chronic myelogenous leukemia will obtain hematologic remission with regression or stabilization of organomegaly following the use of busulfan. Busulfan has been shown to be superior to splenic irradiation with respect to survival times and maintenance of hemoglobin levels, and to be equivalent to irradiation at controlling splenomegaly.

4. Nitrosourea

Nitrosureas, like alkylating agents, inhibit DNA repair proteins. They are used to treat non-Hodgkin's lymphomas, multiple myeloma, malignant melanoma, in addition to brain tumors. A nitrosourea include but is not limited to a carmustine (BCNU), a lomustine (CCNU), a semustine (methyl-CCNU) or a streptozocin. Semustine has been used in such cancers as a primary brain tumor, a stomach or a colon cancer. Stroptozocin has been used to treat diseases such as a malignant pancreatic insulinoma or a malignalnt carcinoid. Streptozocin has been used to treat such cancers as a malignant melanoma, Hodgkin's disease and soft tissue sarcomas.

Carmustine (sterile carmustine) is one of the nitrosoureas used in the treatment of certain neoplastic diseases. It is 1,3 bis (2-chloroethyl)-1-nitrosourea. It is lyophilized pale yellow flakes or congealed mass with a molecular weight of 214.06. It is highly soluble in alcohol and lipids, and poorly soluble in water. Carmustine is administered by intravenous infusion after reconstitution as recommended Although it is generally agreed that carmustine alkylates DNA and RNA, it is not cross resistant with other alkylators. As with other nitrosoureas, it may also inhibit several key enzymatic processes by carbamoylation of amino acids in proteins.

Carmustine is indicated as palliative therapy as a single agent or in established combination therapy with other approved chemotherapeutic agents in brain tumors such as glioblastoma, brainstem glioma, medullobladyoma, astrocytoma, ependymoma, and metastatic brain tumors. Also it has been used in combination with prednisone to treat multiple myeloma. Carmustine has been used in treating such cancers as a multiple myeloma or a malignant melanoma. Carmustine has proved useful, in the treatment of Hodgkin's Disease and in non-Hodgkin's lymphomas, as secondary therapy in combination with other approved drugs in patients who relapse while being treated with primary therapy, or who fail to respond to primary therapy.

Sterile carmustine is commonly available in 100 mg single dose vials of lyophilized material. The recommended dose of carmustine as a single agent in previously untreated patients is about 150 mg/m² to about 200 mg/m² intravenously every 6 weeks. This may be given as a single dose or divided into daily injections such as about 75 mg/m² to about 100 mg/m² on 2 successive days. When carmustine is used in combination with other myelosuppressive drugs or in patients in whom bone marrow reserve is depleted, the doses should be adjusted accordingly. Doses subsequent to the initial dose should be adjusted according to the hematologic response of the patient to the preceding dose. It is of course understood that other doses may be used in the present invention, for example about 10 mg/m², about 20 mg/m², about 30 mg/m², about 40 mg/m², about 50 mg/m², about 60 mg/m², about 70 mg/m², about 80 mg/m², about 90 mg/m² to about 100 mg/m².

Lomustine is one of the nitrosoureas used in the treatment of certain neoplastic diseases. It is 1-(2-chloro-ethyl)-3-cyclohexyl-1 nitrosourea. It is a yellow powder with the empirical formula of C₉H₁₆ClN₃O₂ and a molecular weight of 233.71. Lomustine is soluble in 10% ethanol (about 0.05 mg/mL) and in absolute alcohol (about 70 mg/mL). Lomustine is relatively insoluble in water (less than about 0.05 mg/mL). It is relatively unionized at a physiological pH. Inactive ingredients in lomustine capsules are: magnesium stearate and mannitol.

Although it is generally agreed that lomustine alkylates DNA and RNA, it is not cross resistant with other alkylators. As with other nitrosoureas, it may also inhibit several key enzymatic processes by carbamoylation of amino acids in proteins.

Lomustine may be given orally. Following oral administration of radioactive lomustine at doses ranging from about 30 mg/m² to 100 mg/m², about half of the radioactivity given was excreted in the form of degradation products within 24 hours. The serum half-life of the metabolites ranges from about 16 hours to about 2 days. Tissue levels are comparable to plasma levels at 15 minutes after intravenous administration.

Lomustine has been shown to be useful as a single agent in addition to other treatment modalities, or in established combination therapy with other approved chemotherapeutic agents in both primary and metastatic brain tumors, in patients who have already received appropriate surgical and/or radiotherapeutic procedures. Lomustine has been used to treat such cancers as small-cell lung cancer. It has also proved effective in secondary therapy against Hodgkin's Disease in combination with other approved drugs in patients who relapse while being treated with primary therapy, or who fail to respond to primary therapy.

The recommended dose of lomustine in adults and children as a single agent in previously untreated patients is about 130 mg/m2 as a single oral dose every 6 weeks. In individuals with compromised bone marrow function, the dose should be reduced to about 100 mg/m² every 6 weeks. When lomustine is used in combination with other myelosuppressive drugs, the doses should be adjusted accordingly. It is understood that other doses may be used for example, about 20 mg/m², about 30 mg/m², about 40 mg/m², about 50 mg/m², about 60 mg/m², about 70 mg/M², about 80 mg/m², about 90 mg/m², about 100 mg/m² to about 120 mg/m².

A triazine include but is not limited to such drugs as a dacabazine (DTIC; dimethyltriazenoimidaz olecarboxamide), used in the treatment of such cancers as a malignant melanoma, Hodgkin's disease and a soft-tissue sarcoma.

B. Antimetabolites

Antimetabolites disrupt DNA and RNA synthesis. Unlike alkylating agents, they specifically influence the cell cycle during S phase. They have used to combat chronic leukemias in addition to tumors of breast, ovary and the gastrointestinal tract. Antimetabolites can be differentiated into various categories, such as folic acid analogs, pyrimidine analogs and purine analogs and related inhibitory compounds. Antimetabolites include but are not limited to, 5-fluorouracil (5-FU), cytarabine (Ara-C), fludarabine, gemcitabine, and methotrexate.

1. Folic Acid Analogs

Folic acid analogs include but are not limited to compounds such as methotrexate (amethopterin), which has been used in the treatment of cancers such as acute lymphocytic leukemia, choriocarcinoma, mycosis fungoides, breast, head and neck, lung and osteogenic sarcoma.

2. Pyrimidine Analogs

Pyrimidine analogs include such compounds as cytarabine (cytosine arabinoside), 5-fluorouracil (fluouracil; 5-FU) and floxuridine (fluorode-oxyuridine; FudR). Cytarabine has been used in the treatment of cancers such as acute granulocytic leukemia and acute lymphocytic leukemias. Floxuridine and 5-fluorouracil have been used in the treatment of cancers such as breast, colon, stomach, pancreas, ovary, head and neck, urinary bladder and topical premalignant skin lesions.

5-Fluorouracil (5-FU) has the chemical name of 5-fluoro-2,4 (1H,3H)-pyrimidinedione. Its mechanism of action is thought to be by blocking the methylation reaction of deoxynridylic acid to thymidylic acid. Thus, 5-FU interferes with the synthesis of deoxyribonucleic acid (DNA) and to a lesser extent inhibits the formation of ribonucleic acid (RNA). Since DNA and RNA are essential for cell division and proliferation, it is thought that the effect of 5-FU is to create a thymidine deficiency leading to cell death. Thus, the effect of 5-FU is found in cells that rapidly divide, a characteristic of metastatic cancers.

3. Purine Analogs and Related Inhibitors

Purine analogs and related compounds include, but are not limited to, mercaptopurine (6-mercaptopurine; 6-MP), thioguanine (6-thioguanine; TG) and pentostatin (2-deoxycoformycin). Mercaptopurine has been used in acute lymphocytic, acute granulocytic and chronic granulocytic leukemias. Thrioguanine has been used in the treatment of such cancers as acute granulocytic leukemia, acute lymphocytic leukemia and chronic lymphocytic leukemia. Pentostatin has been used in such cancers as hairy cell leukemias, mycosis fungoides and chronic lymphocytic leukemia.

C. Natural Products

Natural products generally refer to compounds originally isolated from a natural source, and identified has having a pharmacological activity. Such compounds, analogs and derivatives thereof may be, isolated from a natural source, chemically synthesized or recombinantly produced by any technique known to those of skill in the art. Natural products include such categories as mitotic inhibitors, antitumor antibiotics, enzymes and biological response modifiers.

1. Mitotic Inhibitors

Mitotic inhibitors include plant alkaloids and other natural agents that can inhibit either protein synthesis required for cell division or mitosis. They operate during a specific phase during the cell cycle. Mitotic inhibitors include, for example, docetaxel, etoposide (VP 16), teniposide, paclitaxel, taxol, vinblastine, vincristine, and vinorelbine.

Epipodophyllotoxins include such compounds as teniposide and VP16. VP16 is also known as etoposide and is used primarily for treatment of testicular tumors, in combination with bleomycin and cisplatin, and in combination with cisplatin for small-cell carcinoma of the lung. Teniposide and VP16 are also active against cancers such as testis, other lung cancer, Hodgkin's disease, non-Hodgkin's lymphomas, acute granulocytic leukemia, acute nonlymphocytic leukemia, carcinoma of the breast, and Kaposi's sarcoma associated with acquired immunodeficiency syndrome (AIDS).

VP16 is available as a solution (e.g., 20 mg/ml) for intravenous administration and as 50 mg, liquid-filled capsules for oral use. For small-cell carcinoma of the lung, the intravenous dose (in combination therapy) is can be as much as about 100 mg/m² or as little as about 2 mg/m², routinely about 35 mg/m², daily for about 4 days, to about 50 mg/m², daily for about 5 days have also been used. When given orally, the dose should be doubled. Hence the doses for small cell lung carcinoma may be as high as about 200 mg/m² to about 250 mg/m². The intravenous dose for testicular cancer (in combination therapy) is about 50 mg/m² to about 100 mg/m² daily for about 5 days, or about 100 mg/m² on alternate days, for three doses. Cycles of therapy are usually repeated about every 3 to 4 weeks. The drug should be administered slowly (e.g., about 30 minutes to about 60 minutes) as an infusion in order to avoid hypotension and bronchospasm, which are probably due to the solvents used in the formulation.

Taxoids are a class of related compounds isolated from the bark of the ash tree, Taxus brevifolia. Taxoids include but are not limited to compounds such as docetaxel and paclitaxel.

Paclitaxel binds to tubulin (at a site distinct from that used by the vinca alkaloids) and promotes the assembly of microtubules. Paclitaxel is being evaluated clinically; it has activity against malignant melanoma and carcinoma of the ovary. In certain aspects, maximal doses are about 30 mg/m² per day for about 5 days or about 210 mg/m² to about 250 mg/m² given once about every 3 weeks.

Vinca alkaloids are a type of plant alkaloid identified to have pharmaceutical activity. They include such compounds as vinblastine (VLB) and vincristine.

2. Vinblastine

Vinblastine is an example of a plant alkaloid that can be used for the treatment of cancer and precancer. When cells are incubated with vinblastine, dissolution of the microtubules occurs.

Unpredictable absorption has been reported after oral administration of vinblastine or vincristine. At the usual clinical doses the peak concentration of each drug in plasma is approximately 0.4 mM. Vinblastine and vincristine bind to plasma proteins. They are extensively concentrated in platelets and to a lesser extent in leukocytes and erythrocytes.

After intravenous injection, vinblastine has a multi-phasic pattern of clearance from the plasma; after distribution, drug disappears from plasma with half-lives of approximately 1 and 20 hours. Vinblastine is metabolized in the liver to biologically activate derivative desacetylvinblastine. Approximately 15% of an administered dose is detected intact in the urine, and about 10% is recovered in the feces after biliary excretion. Doses should be reduced in patients with hepatic dysfunction. At least a 50% reduction in dosage is indicated if the concentration of bilirubin in plasma is greater than 3 mg/dl (about 50 mM). After a single dose of 0.3 mg/kg of body weight, myelosuppression reaches its maximum in about 7 days to about 10 days. If a moderate level of leukopenia (approximately 3000 cells/mm³) is not attained, the weekly dose may be increased gradually by increments of about 0.05 mg/kg of body weight. In regimens designed to cure testicular cancer, vinblastine is used in doses of about 0.3 mg/kg about every 3 weeks irrespective of blood cell counts or toxicity.

An important clinical use of vinblastine is with bleomycin and cisplatin in the curative therapy of metastatic testicular tumors. Beneficial responses have been reported in various lymphomas, particularly Hodgkin's disease, where significant improvement may be noted in 50 to 90% of cases. The effectiveness of vinblastine in a high proportion of lymphomas is not diminished when the disease is refractory to alkylating agents. It is also active in Kaposi's sarcoma, testis cancer, neuroblastoma, and Letterer-Siwe disease (histiocytosis X), as well as in carcinoma of the breast and choriocarcinoma in women.

Doses of about 0.1 mg/kg to about 0.3 mg/kg can be administered or about 1.5 mg/m² to about 2 mg/m2 can also be administered. Alternatively, about 0.1 mg/m², about 0.12 mg/m², about 0.14 mg/m², about 0.15 mg/m², about 0.2 mg/m², about 0.25 mg/m², about 0.5 mg/m², about 1.0 mg/m², about 1.2 mg/m², about 1.4 mg/m², about 1.5 mg/m², about 2.0 mg/m², about 2.5 mg/m², about 5.0 mg/m², about 6 mg/m², about 8 mg/m², about 9 mg/M², about 10 mg/m², to about 20 mg/m², can be given.

3. Vincristine

Vincristine blocks mitosis and produces metaphase arrest. It seems likely that most of the biological activities of this drug can be explained by its ability to bind specifically to tubulin and to block the ability of protein to polymerize into microtubules. Through disruption of the microtubules of the mitotic apparatus, cell division is arrested in metaphase. The inability to segregate chromosomes correctly during mitosis presumably leads to cell death.

The relatively low toxicity of vincristine for normal marrow cells and epithelial cells make this agent unusual among anti-neoplastic drugs, and it is often included in combination with other myelosuppressive agents.

Unpredictable absorption has been reported after oral administration of vinblastine or vincristine. At the usual clinical doses the peak concentration of each drug in plasma is about 0.4 mM.

Vinblastine and vincristine bind to plasma proteins. They are extensively concentrated in platelets and to a lesser extent in leukocytes and erythrocytes. Vincristine has a multi-phasic pattern of clearance from the plasma; the terminal half-life is about 24 hours. The drug is metabolized in the liver, but no biologically active derivatives have been identified. Doses should be reduced in patients with hepatic dysfunction. At least a 50% reduction in dosage is indicated if the concentration of bilirubin in plasma is greater than about 3 mg/dl (about 50 mM).

Vincristine sulfate is available as a solution (e.g., 1 mg/ml) for intravenous injection. Vincristine used together with corticosteroids is presently the treatment of choice to induce remissions in childhood leukemia; the optimal dosages for these drugs appear to be vincristine, intravenously, about 2 mg/m² of body-surface area, weekly; and prednisone, orally, about 40 mg/m², daily. Adult patients with Hodgkin's disease or non-Hodgkin's lymphomas usually receive vincristine as a part of a complex protocol. When used in the MOPP regimen, the recommended dose of vincristine is about 1.4 mg/m². High doses of vincristine seem to be tolerated better by children with leukemia than by adults, who may experience sever neurological toxicity. Administration of the drug more frequently than every 7 days or at higher doses seems to increase the toxic manifestations without proportional improvement in the response rate. Precautions should also be used to avoid extravasation during intravenous administration of vincristine. Vincristine (and vinblastine) can be infused into the arterial blood supply of tumors in doses several times larger than those that can be administered intravenously with comparable toxicity.

Vincristine has been effective in Hodgkin's disease and other lymphomas. Although it appears to be somewhat less beneficial than vinblastine when used alone in Hodgkin's disease, when used with mechlorethamine, prednisone, and procarbazine (the so-called MOPP regimen), it is the preferred treatment for the advanced stages (III and IV) of this disease. In non-Hodgkin's lymphomas, vincristine is an important agent, particularly when used with cyclophosphamide, bleomycin, doxorubicin, and prednisone. Vincristine is more useful than vinblastine in lymphocytic leukemia. Beneficial response have been reported in patients with a variety of other neoplasms, particularly Wilms' tumor, neuroblastoma, brain tumors, rhabdomyosarcoma, small cell lung, and carcinomas of the breast, bladder, and the male and female reproductive systems.

Doses of vincristine include about 0.01 mg/kg to about 0.03 mg/kg or about 0.4 mg/m² to about 1.4 mg/m² can be administered or about 1.5 mg/m² to about 2 mg/m² can also be administered. Alternatively, in certain embodiments, about 0.02 mg/m², about 0.05 mg/m², about 0.06 mg/m², about 0.07 mg/m², about 0.08 mg/m², about 0.1 mg/m², about 0.12 mg/m², about 0.14 mg/m², about 0.15 mg/m², about 0.2 mg/m², about 0.25 mg/m² can be given as a constant intravenous infusion.

D. Antitumor Antibiotics

Antitumor antibiotics have both antimicrobial and cytotoxic activity. These drugs also interfere with DNA by chemically inhibiting enzymes and mitosis or altering cellular membranes. These agents are not phase specific so they work in all phases of the cell cycle. Thus, they are widely used for a variety of cancers. Examples of antitumor antibiotics include, but are not limited to, bleomycin, dactinomycin, daunorubicin, doxorubicin (Adriamycin), plicamycin (mithramycin) and idarubicin. Widely used in clinical setting for the treatment of neoplasms these compounds generally are administered through intravenous bolus injections or orally.

1. Doxorubicin

Doxorubicin hydrochloride, 5,12-Naphthacenedione, (8s-cis)-10-[(3-amino-2,3,6-trideoxy-a-L-lyxo-hexopyranosyl)oxy]-7,8,9,10-tetrahydro-6,8,11-trihydroxy-8-(hydroxyacetyl)-1-methoxy-hydrochloride (hydroxydaunorubicin hydrochloride, Adriamycin) is used in a wide antineoplastic spectrum. It binds to DNA and inhibits nucleic acid synthesis, inhibits mitosis and promotes chromosomal aberrations.

Administered alone, it is the drug of first choice for the treatment of thyroid adenoma and primary hepatocellular carcinoma. It is a component of 31 first-choice combinations for the treatment of diseases including ovarian, endometrial and breast tumors, bronchogenic oat-cell carcinoma, non-small cell lung carcinoma, stomach, genitourinary, thyroid, gastric adenocarcinoma, retinoblastoma, neuroblastoma, mycosis fungoides, pancreatic carcinoma, prostatic carcinoma, bladder carcinoma, myeloma, diffuse histiocytic lymphoma, Wilms' tumor, Hodgkin's disease, adrenal tumors, osteogenic sarcoma, soft tissue sarcoma, Ewing's sarcoma, rhabdomyosarcoma and acute lymphocytic leukemia. It is an alternative drug for the treatment of other diseases such as islet cell, cervical, testicular and adrenocortical cancers. It is also an immunosuppressant.

Doxorubicin is absorbed poorly and is preferably administered intravenously. The pharmacokinetics are multicompartmental. Distribution phases have half-lives of 12 minutes and 3.3 hours. The elimination half-life is about 30 hours, with about 40% to about 50% secreted into the bile. Most of the remainder is metabolized in the liver, partly to an active metabolite (doxorubicinol), but a few percent is excreted into the urine. In the presence of liver impairment, the dose should be reduced.

In certain embodiments, appropriate intravenous doses are, adult, about 60 mg/m² to about 75 mg/m² at about 21-day intervals or about 25 mg/m² to about 30 mg/m² on each of 2 or 3 successive days repeated at about 3 week to about 4 week intervals or about 20 mg/m² once a week. The lowest dose should be used in elderly patients, when there is prior bone-marrow depression caused by prior chemotherapy or neoplastic marrow invasion, or when the drug is combined with other myelopoietic suppressant drugs. The dose should be reduced by about 50% if the serum bilirubin lies between about 1.2 mg/dL and about 3 mg/dL and by about 75% if above about 3 mg/dL. The lifetime total dose should not exceed about 550 Mg/M2 in patients with normal heart function and about 400 mg/m² in persons having received mediastinal irradiation. In certain embodiments, and alternative dose regiment may comprise about 30 mg/m² on each of 3 consecutive days, repeated about every 4 week. Exemplary doses may be about 10 mg/m², about 20 mg/m², about 30 mg/m², about 50 mg/m², about 100 mg/m², about 150 mg/m², about 175 mg/m², about 200 mg/m², about 225 mg/m², about 250 mg/m², about 275 mg/m², about 300 mg/m², about 350 mg/m², about 400 mg/m², about 425 mg/m², about 450 mg/m², about 475 mg/m², to about 500 mg/m².

2. Daunorubicin

Daunorubicin hydrochloride, 5,12-Naphthacenedione, (8S-cis)-8-acetyl-10-[(3-amino-2,3,6-trideoxy-a-L-lyxo-hexanopyranosyl)oxy]-7,8,9,10-tetrahydro-6,8,11-trihydroxy-10-methoxy-, hydrochloride; also termed cerubidine and available from Wyeth. Daunorubicin (daunomycin; rubidomycin) intercalates into DNA, blocks DAN-directed RNA polymerase and inhibits DNA synthesis. It can prevent cell division in doses that do not interfere with nucleic acid synthesis.

In combination with other drugs it is often included in the first-choice chemotherapy of diseases such as, for example, acute granulocytic leukemia, acute myelocytic leukemia in adults (for induction of remission), acute lymphocytic leukemia and the acute phase of chronic myelocytic leukemia. Oral absorption is poor, and it preferably given by other methods (e.g., intravenously). The half-life of distribution is 45 minutes and of elimination, about 19 hours. The half-life of its active metabolite, daunorubicinol, is about 27 hours. Daunorubicin is metabolized mostly in the liver and also secreted into the bile (about 40%). Dosage must be reduced in liver or renal insufficiencies.

Generally, suitable intravenous doses are (base equivalent): adult, younger than 60 years, about 45 mg/m²/day (about 30 mg/m² for patients older than 60 year.) for about 1 day, about 2 days or about 3 days about every 3 weeks or 4 weeks or about 0.8 mg/kg/day for about 3 days, about 4 days, about 5 days to about 6 days about every 3 weeks or about 4 weeks; no more than about 550 mg/m² should be given in a lifetime, except only about 450 mg/M² if there has been chest irradiation; children, about 25 mg/m once a week unless the age is less than 2 years. or the body surface less than about 0.5 m, in which case the weight-based adult schedule is used. It is available in injectable dosage forms (base equivalent) of about 20 mg (as the base equivalent to about 21.4 mg of the hydrochloride). Exemplary doses may be about 10 mg/m², about 20 mg/m², about 30 mg/m², about 50 mg/m², about 100 Mg/m², about 150 mg/m², about 175 mg/m², about 200 mg/m², about 225 mg/m², about 250 mg/m², about 275 mg/m², about 300 mg/m², about 350 mg/m², about 400 mg/m², about 425 mg/m², about 450 mg/m², about 475 mg/m2, to about 500 mg/m².

3. Mitomycin

Mitomycin (also known as mutamycin and/or mitomycin-C) is an antibiotic isolated from the broth of Streptomyces caespitosus which has been shown to have antitumor activity. The compound is heat stable, has a high melting point, and is freely soluble in organic solvents.

Mitomycin selectively inhibits the synthesis of deoxyribonucleic acid (DNA). The guanine and cytosine content correlates with the degree of mitomycin-induced cross-linking. At high concentrations of the drug, cellular RNA and protein synthesis are also suppressed. Mitomycin has been used in tumors such as stomach, cervix, colon, breast, pancreas, bladder and head and neck.

In humans, mitomycin is rapidly cleared from the serum after intravenous administration. Time required to reduce the serum concentration by about 50% after a 30 mg. bolus injection is 17 minutes. After injection of 30 mg, 20 mg, or 10 mg I.V., the maximal serum concentrations were 2.4 mg/mL, 1.7 mg/mL, and 0.52 mg/mL, respectively. Clearance is effected primarily by metabolism in the liver, but metabolism occurs in other tissues as well. The rate of clearance is inversely proportional to the maximal serum concentration because, it is thought, of saturation of the degradative pathways. Approximately 10% of a dose of mitomycin is excreted unchanged in the unne. Since metabolic pathways are saturated at relatively low doses, the percent of a dose excreted in urine increases with increasing dose. In children, excretion of intravenously administered mitomycin is similar.

4. Actinomycin D

Actinomycin D (Dactinomycin) [50-76-0]; C₆₂H₈₆N₁₂O₁₆ (1255.43) is an antineoplastic drug that inhibits DNA-dependent RNA polymerase. It is often a component of first-choice combinations for treatment of diseases such as, for example, choriocarcinoma, embryonal rhabdomyosarcoma, testicular tumor, Kaposi's sarcoma and Wilms' tumor. Tumors that fail to respond to systemic treatment sometimes respond to local perfilsion. Dactinomycin potentiates radiotherapy. It is a secondary (efferent) immunosuppressive.

In certain specific aspects, actinomycin D is used in combination with agents such as, for example, primary surgery, radiotherapy, and other drugs, particularly vincristine and cyclophosphamide. Antineoplastic activity has also been noted in Ewing's tumor, Kaposi's sarcoma, and soft-tissue sarcomas. Dactinomycin can be effective in women with advanced cases of choriocarcinoma. It also produces consistent responses in combination with chlorambucil and methotrexate in patients with metastatic testicular carcinomas. A response may sometimes be observed in patients with Hodgkin's disease and non-Hodgkin's lymphomas. Dactinomycin has also been used to inhibit immunological responses, particularly the rejection of renal transplants.

Half of the dose is excreted intact into the bile and 10% into the urine; the half-life is about 36 hours. The drug does not pass the blood-brain barrier. Actinomycin D is supplied as a lyophilized powder (0/5 mg in each vial). The usual daily dose is about 10 mg/kg to about 15 mg/kg; this is given intravenously for about 5 days; if no manifestations of toxicity are encountered, additional courses may be given at intervals of about 3 weeks to about 4 weeks. Daily injections of about 100 mg to about 400 mg have been given to children for about 10 days to about 14 days; in other regimens, about 3 mg/kg to about 6 mg/kg, for a total of about 125 mg/kg, and weekly maintenance doses of about 7.5 mg/kg have been used. Although it is safer to administer the drug into the tubing of an intravenous infusion, direct intravenous injections have been given, with the precaution of discarding the needle used to withdraw the drug from the vial in order to avoid subcutaneous reaction. Exemplary doses may be about 100 mg/m², about 150 mg/m², about 175 mg/m², about 200 mg/m², about 225 mg/m², about 250 mg/m², about 275 mg/m², about 300 mg/m², about 350 mg/m², about 400 mg/m², about 425 mg/m², about 450 mg/m², about 475 mg/m², to about 500 mg/m².

5. Bleomycin

Bleomycin is a mixture of cytotoxic glycopeptide antibiotics isolated from a strain of Streptomyces verticillus. Although the exact mechanism of action of bleomycin is unknown, available evidence would seem to indicate that the main mode of action is the inhibition of DNA synthesis with some evidence of lesser inhibition of RNA and protein synthesis.

In mice, high concentrations of bleomycin are found in the skin, lungs, kidneys, peritoneum, and lymphatics. Tumor cells of the skin and lungs have been found to have high concentrations of bleomycin in contrast to the low concentrations found in hematopoietic tissue. The low concentrations of bleomycin found in bone marrow may be related to high levels of bleomycin degradative enzymes found in that tissue.

In patients with a creatinine clearance of greater than about 35 mL per minute, the serum or plasma terminal elimination half-life of bleomycin is approximately 115 minutes. In patients with a creatinine clearance of less than about 35 mL per minute, the plasma or serum terminal elimination half-life increases exponentially as the creatinine clearance decreases. In humans, about 60% to about 70% of an administered dose is recovered in the urine as active bleomycin. In specific embodiments, bleomycin may be given by the intramuscular, intravenous, or subcutaneous routes. It is freely soluble in water. Because of the possibility of an anaphylactoid reaction, lymphoma patients should be treated with two units or less for the first two doses. If no acute reaction occurs, then the regular dosage schedule may be followed.

In preferred aspects, bleomycin should be considered a palliative treatment. It has been shown to be useful in the management of the following neoplasms either as a single agent or in proven combinations with other approved chemotherapeutic agents in squamous cell carcinoma such as head and neck (including mouth, tongue, tonsil, nasopharynx, oropharynx, sinus, palate, lip, buccal mucosa, gingiva, epiglottis, larynx), esophagus, lung and genitourinary tract, Hodgkin's disease, non-Hodgkin's lymphoma, skin, penis, cervix, and vulva. It has also been used in the treatment of lymphomas and testicular carcinoma.

Improvement of Hodgkin's Disease and testicular tumors is prompt and noted within 2 weeks. If no improvement is seen by this time, improvement is unlikely. Squamous cell cancers respond more slowly, sometimes requiring as long as 3 weeks before any improvement is noted.

E. Hormones and Antagonists

Hormonal therapy may also be used in conjunction with the present invention and/or in combination with any other cancer therapy or agent(s). The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

1. Adrenocorticosteroids

Corticosteroid hormones are useful in treating some types of cancer (e.g., non-Hodgkin's lymphoma, acute and chronic lymphocytic leukemias, breast cancer, and multiple myeloma). Though these hormones have been used in the treatment of many non-cancer conditions, they are considered chemotherapy drugs when they are implemented to kill or slow the growth of cancer cells. Corticosteroid hormones can increase the effectiveness of other chemotherapy agents, and consequently, they are frequently used in combination treatments. Prednisone and dexamethasone are examples of corticosteroid hormones.

2. Other Hormones and Antagonists

Progestins such as hydroxyprogesterone caproate, medroxyprogesterone acetate, and megestrol acetate have been used in cancers of the endometrium and breast. Estrogens such as diethylstilbestrol and ethinyl estradiol have been used in cancers such as breast and prostate. Antiestrogens such as tamoxifen have been used in cancers such as breast. Androgens such as testosterone propionate and fluoxymesterone have also been used in treating breast cancer. Antiandrogens such as flutamide have been used in the treatment of prostate cancer. Gonadotropin-releasing hormone analogs such as leuprolide have been used in treating prostate cancer. U.S. Pat. No. 4,418,068, incorporated herein by reference, discloses antiestrogenic and antiandrogenic benzothiophenes, such as, for example, 6-hydroxy-2-(4-hydroxyphenyl)-3-[4-(2-piperidinoethoxy)benzoyl]benzo[b]thiophene, and esters, ethers, and salts thereof for the treatment of cancers such as prostate and breast cancer.

F. Miscellaneous Agents

Some chemotherapy agents do not qualify into the previous categories based on their activities. They include, but are not limited to, platinum coordination complexes, anthracenedione, substituted urea, methyl hydrazine derivative, adrenalcortical suppressant, amsacrine, L-asparaginase, and tretinoin. It is contemplated that they are included within the compositions and methods of the present invention for use in combination therapies.

1. Platinum Coordination Complexes

Platinum coordination complexes include such compounds as carboplatin and cisplatin (cis-DDP). Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes, such as for example, intravenous, subcutaneous, intratumoral or intraperitoneal injection. Cisplatin can be used alone or in combination with other agents, with efficacious doses used in clinical applications of about 15 mg/m² to about 20 mg/m² for 5 days every three weeks for a total of three courses being contemplated in certain embodiments. Doses may be, for example, about 0.50 mg/m², about 1.0 mg/m², about 1.50 mg/m², about 1.75 mg/m², about 2.0 mg/m², about 3.0 mg/m², about 4.0 mg/m², about 5.0 mg/m², to about 10 mg/m².

2. Other Agents

An anthracenedione such as mitoxantrone has been used for treating acute granulocytic leukemia and breast cancer. A substituted urea such as hydroxyurea has been used in treating chronic granulocytic leukemia, polycythemia vera, essential thrombocytosis and malignant melanoma. A methyl hydrazine derivative such as procarbazine (N-methylhydrazine, MIH) has been used in the treatment of Hodgkin's disease. An adrenocortical suppressant such as mitotane has been used to treat adrenal cortex cancer, while aminoglutethimide has been used to treat Hodgkin's disease.

G. Radiotherapeutic Agents

Radiotherapeutic agents include radiation and waves that induce DNA damage for example, yirradiation, X-rays, proton beam irradiation, UV-irradiation, microwaves, electronic emissions, radioisotopes, and the like. Therapy may be achieved by irradiating the localized tumor site with the above described forms of radiation. It is most likely that all of these agents effect a broad range of damage DNA, on the precursors of DNA, the replication and repair of DNA, and the assembly and maintenance of chromosomes.

Radiotherapeutic agents and methods of administration, dosages, etc. are well known to those of skill in the art, and may be combined with the invention in light of the disclosures herein. For example, dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

H. Immunotherapeutic Agents

An immunotherapeutic agent generally relies on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (e.g., a chemotherapeutic, a radionuclide, a ricin A chain, a cholera toxin, a pertussis toxin, etc.) and serve merely as a targeting agent. Such antibody conjugates are called immunotoxins, and are well known in the art (see U.S. Pat. No. 5,686,072, U.S. Pat. No. 5,578,706, U.S. Pat. No. 4,792,447, U.S. Pat. No. 5,045,451, U.S. Pat. No. 4,664,911, and U.S. Pat. No. 5,767,072, each incorporated herein by reference). Alternatively, the effector may be a lymphocyte carrying a surface molecule that interacts, either directly or indirectly, with a tumor cell target. Various effector cells include cytotoxic T cells and NK cells.

In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

1. Immune Stimulators

In a specific aspect of immunotherapy is to use an immune stimulating molecule as an agent, or more preferably in conjunction with another agent, such as for example, a cytokines such as for example IL-2, IL-4, IL-12, GM-CSF, tumor necrosis factor; interferons alpha, beta, and gamma; F42K and other cytokine analogs; a chemokine such as for example MIP-1, MIP-lbeta, MCP-1, RANTES, IL-8; or a growth factor such as for example FLT3 ligand.

One particular cytokine contemplated for use in the present invention is tumor necrosis factor. Tumor necrosis factor (TNF; Cachectin) is a glycoprotein that kills some kinds of cancer cells, activates cytokine production, activates macrophages and endothelial cells, promotes the production of collagen and collagenases, is an inflammatory mediator and also a mediator of septic shock, and promotes catabolism, fever and sleep. Some infectious agents cause tumor regression through the stimulation of TNF production. TNF can be quite toxic when used alone in effective doses, so that the optimal regimens probably will use it in lower doses in combination with other drugs. Its immunosuppressive actions are potentiated by gamma-interferon, so that the combination potentially is dangerous. A hybrid of TNF and interferon-a also has been found to possess anti-cancer activity.

Another cytokine specifically contemplate is interferon alpha. Interferon alpha has been used in treatment of hairy cell leukemia, Kaposi's sarcoma, melanoma, carcinoid, renal cell cancer, ovary cancer, bladder cancer, non-Hodgkin's lymphomas, mycosis fungoides, multiple myeloma, and chronic granulocytic leukemia.

2. Passive Immunotherapy

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

Preferably, human monoclonal antibodies are employed in passive immunotherapy, as they produce few or no side effects in the patient. However, their application is somewhat limited by their scarcity and have so far only been administered intralesionally. For example, human monoclonal antibodies to ganglioside antigens have been administered intralesionally to patients suffering from cutaneous recurrent melanoma (Irie & Morton, 1986). Regression was observed in six out of ten patients, following, daily or weekly, intralesional injections. In another study, moderate success was achieved from intralesional injections of two human monoclonal antibodies (Irie et al., 1989).

It may be favorable to administer more than one monoclonal antibody directed against two different antigens or even antibodies with multiple antigen specificity. Treatment protocols also may include administration of lymphokines or other immune enhancers (Bajorin et al., 1988).

3. Active Immunotherapy

In active immunotherapy, an antigenic peptide, polypeptide or protein, or an autologous or allogenic tumor cell composition or “vaccine” is administered, generally with a distinct bacterial adjuvant (Ravindranath & Morton, 1991; Morton & Ravindranath, 1996; Morton et al., 1992; Mitchell et al., 1990; Mitchell et al., 1993). In melanoma immunotherapy, those patients who elicit high IgM response often survive better than those who elicit no or low IgM antibodies (Morton et al., 1992). IgM antibodies are often transient antibodies and the exception to the rule appears to be anti-ganglioside or anticarbohydrate antibodies.

4. Adoptive Immunotherapy

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989). To achieve this, one would administer to an animal, or human patient, an immunologically effective amount of activated lymphocytes in combination with an adjuvant-incorporated anigenic peptide composition as described herein. The activated lymphocytes will most preferably be the patient's own cells that were earlier isolated from a blood or tumor sample and activated (or “expanded”) in vitro. This form of immunotherapy has produced several cases of regression of melanoma and renal carcinoma, but the percentage of responders were few compared to those who did not respond.

I. Other Biological Agents

It is contemplated that other agents may be used in combination with the present invention to improve the therapeutic efficacy of treatment. These additional agents include, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents such as for example, hyperthermia.

It is further contemplated that the upregulation of cell surface receptors or their ligands such as Fas/Fas ligand, DR4 or DR5/TRAIL would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population.

In other embodiments, cytostatic or differentiation agents can be used in combination with the present invention to improve the anti-hyerproliferative efficacy of the treatments.

Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as, for example, the antibody c225, could be used in combination with the present invention to improve the treatment efficacy.

Another form of therapy for use in conjunction with the present invention and/or other agent(s) includes hyperthennia, which is a procedure in which a patient's tissue is exposed to high temperatures (up to 106° F.). External or internal heating devices may be involved in the application of local, regional, or whole-body hyperthernia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

XII. Treatment of Lung Disease and Other Diseases

The pharmaceutical compositions that can be delivered by the formulations and method of the instant invention can be used to treat a variety of diseases that affect the lungs. These diseases includes diseases of the airways such as asthma, bronchilitis, cystic fibrosis, bronchiectasis, chronic obstructive pulmonary disease (COPD) which includes asthmatic bronchitis, chronic bronchitis (with normal airflow), chronic obstructive bronchitis, bullous disease, and emphysema, as well as other diseases characterized by structural changes in the airways that limit or obstruct the flow of air in or out of the lungs. Other diseases that can be treated using the aerosol delivery formulation of the current invention include diseases of the pleura, or the membrane that surrounds the lungs, such as infections like pneumonia and tuberculosis and other diseases that are characterized by air or fluid accumulating in the pleural space. Still other diseases include the diseases of the interstitium, the space between the tissues of the lungs which cause the lungs to stiffen and scar and can be caused by drugs, poisons, infections, or radiation. Disorders of the gas exchange or blood circulation in the lungs can also be treated using the delivery method of the current invention. These diseases include pulmonary edema, pulmonary embolism, respiratory failure, and pulmonary hypertension (http://www.4woman.gov/x/faq/lung_disease.htm).

Diseases that affect other areas of the pulmonary system and mucosa can also be treated using pharmaceutical compositions delivered by the method of the instant invention. These diseases include but are not limited to rhinitis, sinusitis, chronic sinusitis, celiac disease, diabetes and hypertension. The methods and formulations of this invention can also be used to treat diseases which are commonly treated by oral administration, but where the therapeutic agent is easily destroyed in digestive track.

XIII. Treatment of Disease via the Blood Stream

It is an aspect of the current invention that the aerosol delivery of pharmaceutical compositions of the current invention can be used to treat diseases and cancers via aerosol delivery to the blood stream. The surface area of the lungs is similar to the size of a tennis court in a normal adult, and the tissue is highly adsorptive. Adsorption into the bloodstream is sometimes faster than subcutaneous injections. Although adsorption in the lungs is rapid, it tends to be less efficient than injections. For example, the bioavailability of aerosolized insulin is 10-15% of injected insulin (Henry, 2000). Therefore, the amount injected may be adjusted, or the pharmaceutical agent may be altered for more efficient delivery.

The aerosol delivery of pharmaceutical compositions can be used in place of oral delivery for many therapeutic agents wherein the agent's effectiveness is reduced or destroyed by deleterious interactions between the therapeutic agent and the digestive track.

XIV. Diagnostic Agents

The invention also relates to an in vivo method of imaging a disease state, such as a cancerous tumor, using aerosol delivery of a diagnostic agent. Specifically, aerosol delivery of the current invention can be used to deliver diagnostic agents to the lungs, blood, or tissue. The particles are useful for diagnosis of pulmonary function abnormalities, structural abnormalities, tumors, blockages, and mismatches in ventilation and perfusion. This method involves administering to a subject an imaging-effective amount of a diagnostic agent and a pharmaceutically effective carrier and detecting the binding of the diagnostic agent to the diseased tissue. The term “in vivo imaging” refers to any method which permits the detection of a diagnostic agent delivered with the aerosol formulation of the present invention that specifically binds to a diseased tissue located in the subject's body. A “subject” is a mammal, preferably a human. An “imaging effective amount” means that the amount of the detectably-labeled monoclonal antibody, or fragment thereof, administered is sufficient to enable detection of binding of the monoclonal antibody or fragment thereof to the diseased tissue.

The diagnostic agent can be any biocompatible or pharmacologically acceptable agent which is trapped into the pores of the aerosol formulation or incorporated into the polymeric or lipid material. The biocompatible or pharmacologically acceptable agent can be a gas such as argon or nitrogen or an imaging agent including the commercially available agents for use in positron emission tomography, computer assisted tomography, single photon emission computerized tomography, x-ray, fluoroscopy, and magnetic resonance imaging.

A factor to consider in selecting a radionuclide for in vivo diagnosis is that the half-life of a nuclide be long enough so that it is still detectable at the time of maximum uptake by the target, but short enough so that deleterious radiation upon the host, as well as background, is minimized. Ideally, a radionuclide used for in vivo imaging will lack a particulate emission, but produce a large number of photons in a 140-2000 keV range, which may be readily detected by conventional gamma cameras.

A radionuclide may be bound to a polypeptide either directly or indirectly by using an intermediary functional group. Intermediary functional groups which are often used to bind radioisotopes which exist as metallic ions to antibody are diethylenetriaminepentaacetic acid (DTPA) and ethylene diaminetetracetic acid (EDTA). Examples of metallic ions suitable for use in this invention are ^{99m}Tc, ¹²³I, ¹¹¹In, ¹³¹I, ⁹⁷Ru, ⁶⁷Cu, ⁶⁷Ga, ¹²⁵I, ⁶⁸Ga, ⁷²As, ⁸⁹Zr, and ²⁰¹Tl.

In accordance with this invention, the aerosol formulation comprising a diagnostic agent may be labeled by any of several techniques known to the art. The methods of the present invention also may use paramagnetic isotopes for purposes of in vivo detection. Elements particularly useful in Magnetic Resonance Imaging (“MRI”) include ¹⁵⁷Gd, ⁵⁵Mn, ¹⁶²Dy, ⁵²Cr, and ⁵⁶Fe.

After a sufficient time has lapsed after aerosol administration for the diagnostic agent to bind with the diseased tissue, for example 30 min to 48 h, the area of the subject under investigation is examined by routine imaging techniques such as MRI, SPECT, planar scintillation imaging and emerging imaging techniques, as well. The exact protocol will necessarily vary depending upon factors specific to the patient, as noted above, and depending upon the body site under examination, method of administration and type of label used; the determination of specific procedures would be routine to the skilled artisan. The distribution of the bound radioactive isotope and its increase or decrease with time is then monitored and recorded. By comparing the results with data obtained from studies of clinically normal individuals, the presence and extent of the diseased tissue may be determined.

XV. Pharmaceutical Preparations

Pharmaceutical compositions of the present invention comprise an effective amount of one or more pharmaceutically acceptable composition, or compositions and or an additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards. The dosage, formulation and delivery may be selected for a particular therapeutic application such as those described by Gonda (1990).

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutically acceptable compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/body weight, about 75 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The pharmaceutically acceptable composition or component of such a composition or additional agent may be formulated in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

The composition must be stable under the conditions of manufacture, storage and delivery and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

XVI. Kits

Any of the compositions described herein may be comprised in a kit. In a non-limiting example, a polycationic polymer, a cationic lipid, PEG, PEI, a pharmaceutically acceptable composition and/or an additional agent, may be comprised in a kit. The kits will thus comprise, in suitable container means, an aerosol formulation comprising one or more components of the current invention and/or an additional agent of the present invention. The kit may also contain means for delivering the aerosol formulation such as an inhaler or other pressurized aerosol canister.

The kits may comprise a suitably aliquoted a polycationic polymer, a cationic lipid, PEG, PEI, a pharmaceutically acceptable composition and/or additional agent compositions of the present invention, whether labeled or unlabeled, as may be used to prepare a standard curve for a detection assay. The therapeutic components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the aerosol formulation, one or more components of an aerosol formulation, additional agents, and any other reagent containers in close confinement for commercial sale. Such containers may include injection or blow-molded plastic containers into which the desired vials are retained.

Therapeutic kits of the present invention are kits aerosol formulations comprising a pharmaceutically acceptable composition and/or an additional agent. Such kits will generally contain, in suitable container means, a pharmaceutically acceptable formulation of the pharmaceutically composition, a component of a aerosol formulation and/or an additional agent in a pharmaceutically acceptable formulation. The kit may have a single container means, and/or it may have distinct container means for each compound.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means.

The container means will generally include at least one vial, test tube, flask, bottle, syringe and/or other container means, into which a pharmaceutically acceptable formulation of the pharmaceutically composition, a component of an aerosol formulation and/or an additional agent formulation are placed, preferably, suitably allocated. The kits may also comprise a second container means for containing a sterile, pharmaceutically acceptable buffer and/or other diluent.

The kits of the present invention will also typically include a means for containing the vials in close confinement for commercial sale, such as, e.g., injection and/or blow-molded plastic containers into which the desired vials are retained.

Irrespective of the number and/or type of containers, the kits of the invention may also comprise, and/or be packaged with an instrument for assisting with the delivery of the aerosol formulation within the body of an animal. Such an instrument may be an inhaler, air compressor and/or any such medically approved delivery vehicle.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

When ratios are given as, for example, 2:1, it is understood that, because errors occur in both the formulation and measurement, this ratio encompasses a range of 15% around the given value.

As used herein the specification, “a” or “an” may mean one or more. As used herein in the claim(s), when used in conjunction with the word “comprising”, the words “a” or “an” may mean one or more than one. As used herein “another” may mean at least a second or more.

The following abbreviations are used in the Figures and Examples:

| | |
|---|---|
| Z1 | Protamine:PEI:PEG:DPEPC, with a preferred weight ratio of 10:1:5:2 |
| Z2 | Polylysine:PEG, with a preferred weight ratio of 10:15 |
| Z3 | Protamine:PEG, with a preferred weight ratio of 10:4 |
| Z4 | Polylysine:PEI:PEG:DPEPC, with a preferred weight ratio of |
| | 10:1:16:2 |
| Z5 | Protamine:Polylysine:PEG, with a prefered weight ratio of 10:7:18 |

wherein PEG is polyethyleneglycol, PEI is polyethylenimine, and DPEPC is dipalmitoyl-glyceroethylphosphocholine. Lipofectamine was purchased from Gibco Life Technologies, and G67 liposome formulation obtained from Genzem Co.

### XVII. EXAMPLES

Other objects, features and advantages of the present invention will become apparent from the following examples. It should be understood, however, that the detailed description and the specific examples, while indicating preferred embodiments of the invention, are given by way of illustration only, since various changes and modifications within the spirit and scope of the invention will become apparent to those skilled in the art from this detailed description and examples.

### Example 1

### Phospholipid Reduces the Cytotoxicity of PEI in Culture of Human Normal Bronchial Epithelium Cells

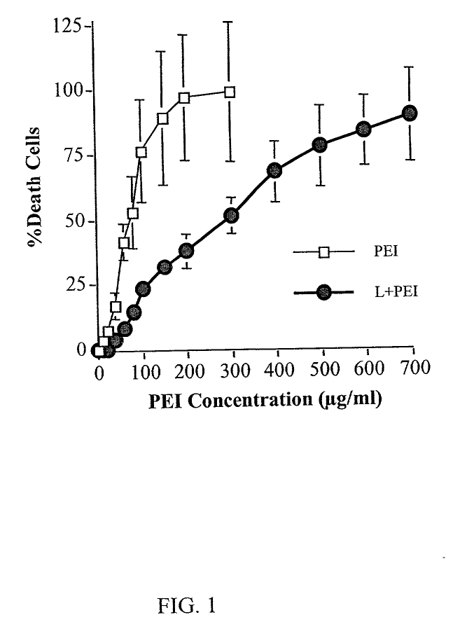

In general cationic polymer such as polyethylenimine (PEI) has relatively higher transfection efficiency and also higher toxicity than most lipid based transfection agents. In order to reduce the toxicity and enhance or at least maintain the transfection efficiency of cationic polymer, the cationic phospholipid DPEPC was used to combine with PEI in a 1:2 weight ratio. Briefly, DPEPC was dissolved in chloroform and dried into a thin film on the wall of flask on a rotary evaporator. The lipid-PEI combination (L-PEI) was obtained by hydrating the lipid thin film with PEI in phosphate buffer solution (PBS). Twenty-four hours later the L-PEI was filtrated by passing a membrane filter with pore size of 0.22 μm. Different amount of suspension containing PEI alone or L-PEI were added onto the human normal bronchial epithelial cells (HNBE) cultured on 6-well plates. Forty-eight hours later, the cells were harvested and the viable cells were counted after Trypan blue staining. The control was non-treated HNBE cells. The data shown in FIG. 1 demonstrates that the cytotoxicity of L-PEI is about 4-fold lower than that of PEI alone (ID50 L+PEI:ID50 PEI=3.8:1). The data in this figure is mean ±one standard deviation from 3 independent experiments.

### Example 2

### Transfection Efficiency of Lipid-PEI combination and PEI

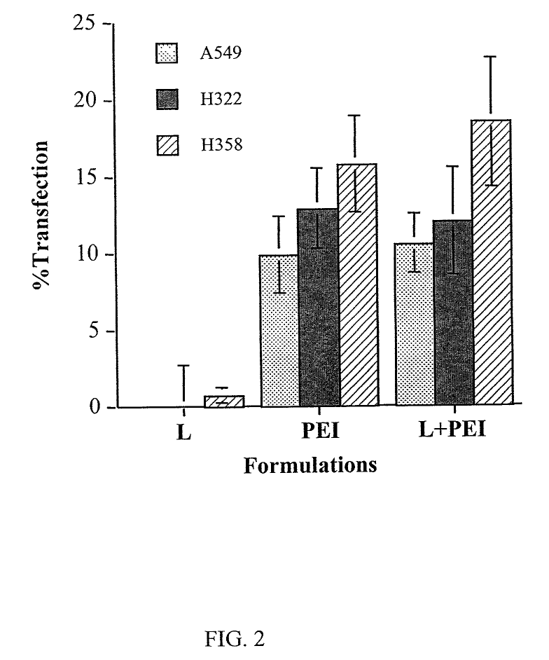

In order to know whether the lipid will affect the transfection efficiency of cationic polymer in the lipid-polymer combination, the transfection efficiency of L-PEI was determined on different cell lines and compared with lipid or PEI alone. The cationic lipid (DPEPC liposomes), PEI, and the L-PEI(1:2 w/w) were complexed with green-fluorescence-protein expression plasmid (GFP) as their own optimal ratio. The three formulations were then generated into aerosol through an air compressor and a nebulizer (40 μg of DNA/ml), separately. The airflow to generate the aerosol was fixed at 4.0 PSI. The aerosols were generated for more than 10 min. The aerosol fog was condensed in a test-tube through a tube that was connected on the output side of the nebulizer. After 10 minutes, about 80 μl of the condensed aerosol liquid from each formulation was collected in sterile test tubes. The condensed aerosol liquids (50 μl/well) were used to transfect human non-small cell lung carcinoma cell lines A549, H322, and H358 cultured in 6-well plates. The optimal transfection conditions for each formulation were used. Forty hours later, the transfection efficiency (% Transfection) was determined by counting percentage of the fluorescent cells under a fluorescence microscope. Each sample was counted 6 random fields with >200 cells/field. FIG. 2 demonstrates that aerosolized lipid-PEI combination of the current invention is better able to transfect human non-small cell lung carcinoma cell lines than the same lipid without PEI or PEI without the cationic lipid, having a much higher transfection efficiency than the lipid alone and similar transfection efficiency to PEI alone. The data is mean ±one standard deviation from 3 independent experiments.

### Example 3

### Transfection Efficiency of a Lipid Combined with Multiple Cationic Polymers

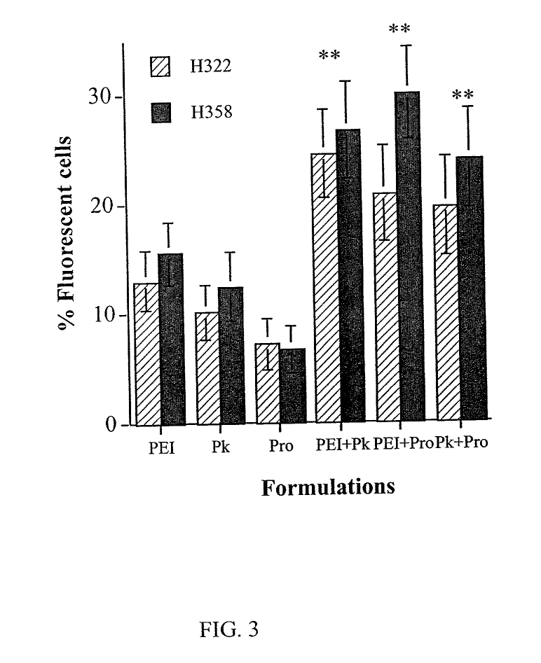

The efficiency of nonviral gene delivery depends on the characteristics of the complex of DNA and its delivery system. While various chemical and stereotype structures of plasmid DNA result in a heterogeneous formation of the complex of DNA and delivery system, e.g., DNA-polymer or DNA-lipid. In order to form more efficient complex, a combination of multiple cationic polymers and endocytosis digested agents may be needed. The inventors designed the formulations composed of multiple cationic polymers and phospholipid. Any two of the three cationic polymers: polylysine (Pk), protamine (Pro) and polyethylenimine (PEI) were combined with their optimal ratio known by the preliminary tests. They were compared with the single polymer formulation in transfection of GFP into human non-small cell lung carcinoma cell lines (H322 and H358). The transfection procedure was the same as described in Example 2. As shown in FIG. 3, under the optimal transfection conditions, the formulations composing of multiple cationic polymers, e.g., PEI+Pk (named as Zl) and PEI+Pro (named as Z4) have significantly higher transfection efficiency than any single polymer formulation. The data is mean ±one standard deviation from 3 independent experiments.

### Example 4

### The Stability of Multiple and Single Cationic Polymer Formulations in Aerosol

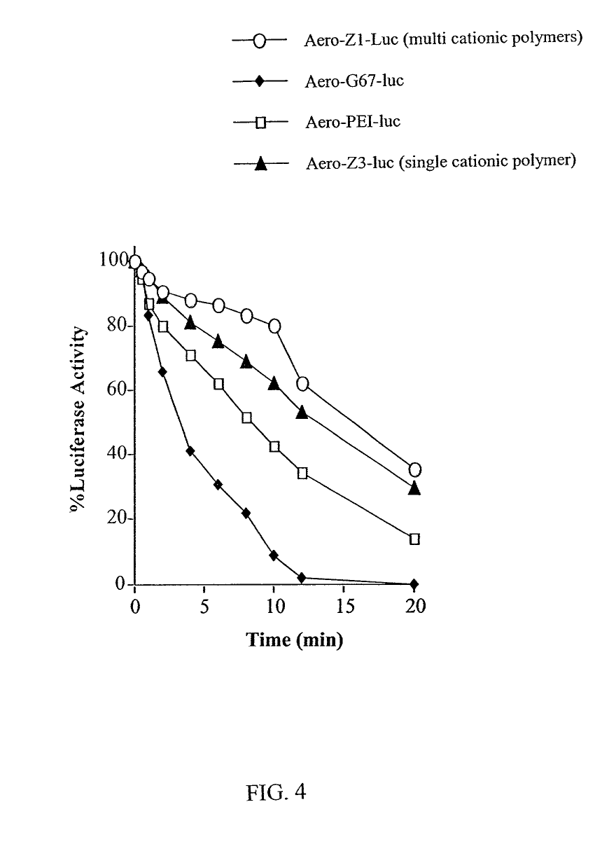

Example 3 demonstrated that multiple cationic polymer formulations are better in transfecting cells in vitro. However, whether the formulations will maintain their transfection efficiency after aerosolization was tested. In this example, stability of formulations that underwent aerosolization was tested. One of the most efficient liposome gene delivery system G67 currently used in clinical trial for aerosol gene delivery to treat cystic fibrosis and the most widely used cationic polymer PEI were used as comparison. The formulations of Z1 (containing multiple cationic polymers), Z3 (containing single cationic polymer), G67 (liposome formulation), or PEI was complexed with a luciferase expression plasmid driven by CMV promoter at the optimal ratios, respectively. Z1-luc, Z3-luc, G67-luc and PEI-luc represent the formulations containing protamine+PEI-luciferase, protamine-luciferase, liposome G67-luciferase, or PEI-luciferase complexes, respectively. Each complex was put in a nebulizer to generate aerosol with 40 pg of DNA in 1.0 ml. The airflow to generate aerosol was fixed at 4.0 PSI. At each designed time point, e.g. 2, 4, 6, 8, 10, 12, and 20 minutes, about 80 μl of condensed aerosol liquid from each formulation was collected in sterile test-tube, then it was used to transfect H358 cells in 6-well plate with 50 μl of the condensed liquid/well. After 24 hours the luciferase activity in one million cells of each well was determined. The luciferase activity in the cells transfected with non-aerosolized formulation (or aerosolized 0 min) was defined as 100%. The results in FIG. 4 indicate that the formulation containing multiple cationic polymers has undergone the aerosolization, therefore it has higher transfection efficiency than those of single polymer or liposome formulation at all time points in the test.

### Example 5

### Delivery of Functional Genes with Aerosolized Formulations Containing Multiple Cationic Polymers in Cultured Cells

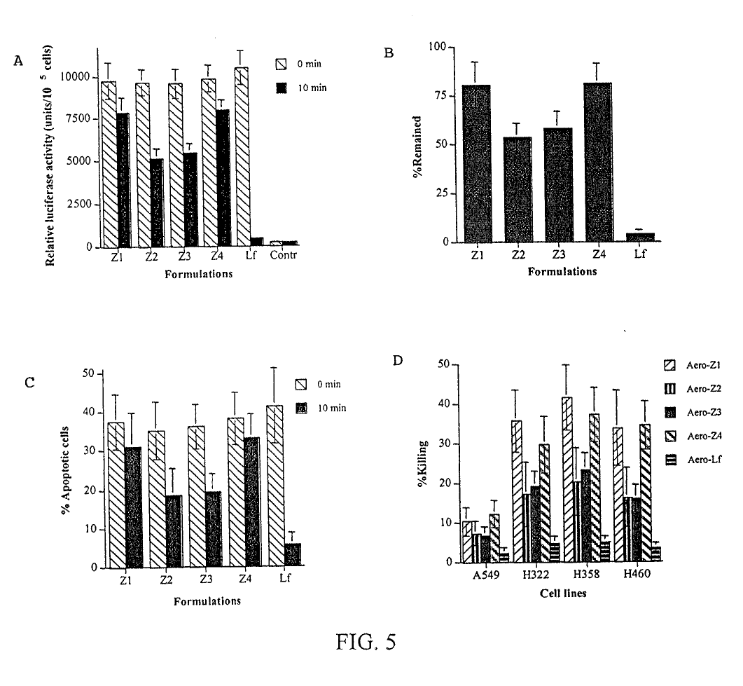

In order to confirm gene-delivery function of the invented formulations by transfecting a functional gene, wild-type p53 was used as prototype gene to deliver into the cultured human lung cancer cell lines. The formulations of Z1, Z4 (containing multiple cationic polymers), Z2, Z3 (containing single cationic polymer), or Lf-(lipofectamine, a commercial cationic liposome formulation) were complexed with CMV promoter driven wild-type p53 gene expression plasmid and p21 promoter driven luciferase expression plasmid (p53:luc=1:1 mol/mol). The complex formulations were put in the reservoir of the nebulizer, separately, with 40 μg of each DNA in 1.0 ml. The airflow to generate aerosol was fixed at 4.0 PSI. The output pipe of the nebulizer was connected to a sterile test-tube on ice to collect the condensed aerosolized liquid. At 0 and 10 minutes, about 80 μl of condensed liquid from each formulation was collected, then it was used to transfect H358 cells in 6-well plate with 50 μL of the condensed liquid/well. Forty hours later the luciferase activity in each well was determined (FIG. 5A). It was found that the transfection efficiencies of all formulations before aerosolization are similar (the difference was <4%). However, after 10 minutes aerosolization the transfection efficiencies are significantly different. In FIG. 5B the percent of luciferase activity remained of each formulation at 10 minutes was presented based on the same experimental results showed in FIG. 5A. The results indicate that the formulations containing multiple cationic polymers are stable under the aerosolization. They are able to remain >80% ability to transfect wild-type p53 gene into the cells. Therefore, there is significant amount of p21 promoter driven luciferase gene expression induced by the transfected p53. However, the single cationic polymer formulations only can remain about 50% ability of transfection, while the liposome formulation almost lost all transfection function after 10 minutes aerosolization.

In another experiment (FIG. 5C), the apoptotic function of wild-type p53 gene delivered by the aerosolized formulations containing multiple cationic polymers to the lung cancer cells was determined. The experimental procedure was similar as that described above. Two days after transfection, the apoptotic cells were determined by Tunel assay. The control was non-treated cells. A similar experiment was repeated by transfecting p53 gene with the aerosolized formulations to multiple cancer cell lines (A549, H322, H358, and H460), but the termination assay used was counting viable cells (FIG. 5D). The results indicate that the aerosolized formulations containing multiple cationic polymers not only effectively delivered the gene into the lung cancer cells in vitro, but also enhanced the expression of the typical functions of p53 gene such as transcription factor, apoptosis induction. These functions can be used for cancer gene therapy. The data shown in this example is mean ±SD from 3 independent experiments.

### Example 6

### Aerosol Efficiency in Mice

In order to estimate the aerosol dose and increase the efficiency of aerosol administration, the amount of aerosol droplets breathed into lung by animal should be determined. The percent of the aerosol breathed in mouse lung in total administered dose is defined as “aerosol efficiency in mice”; this does not take the gene transfection efficiency into consideration. The experiment was designed by labeling formulations with a fluorescence dye and administering the labeling aerosol to mice and measuring the administered dose and the amount of the fluorescence dye in the lungs of mice. Briefly, an equal amount of fluorescent dye calcein was mixed with formulations Z1, Z2, Z3 and Z4. To increase the aerosol breath efficiency the ICR mice (19-21 g) were put in a specially designed restriction cage, name YZ restriction cage, and then the mice in different group were given the same dose of aerosol of each formulation containing calcien, separately. The airflow rate of aerosol was fixed at 4.0 PSI. When the aerosol was given for 2, 6, or 10 minutes, five mice from each group were terminated, their lungs were resected, and the calcein concentrations were immediately quantitatively determined by a fluorescence-spectrophotometer. The administered doses were determined by measuring the initial amount of the formulation and remaining amount of the formulation in the nebulizer reservoir. The results in Table 7 show that aerosol efficiency in mice was about 3% and the difference between the formulations tested is not significant above the error. The data of each time point for each formulation shown in Table 7 is mean +one standard deviation from 5 mice.

TABLE 7
The Aerosol Efficiency in Mice

| Aerosolization time (min) | 2 | 6 | 10 |
|---|---|---|---|
| Accumulated Z1 breathed | 0.46 ± 0.08 | 1.70 ± 0.35 | 3.04 ± 0.72 |
| by mice (% of initial) | | | |
| Accumulated Z2 breathed | 0.42 ± 0.10 | 1.47 ± 0.41 | 2.82 ± 0.75 |
| by mice (% of initial) | | | |
| Accumulated Z3 breathed | 0.45 ± 0.19 | 1.55 ± 0.34 | 2.89 ± 0.97 |
| by mice (% of initial) | | | |
| Accumulated Z4 breathed | 0.51 ± 0.11 | 1.63 ± 0.38 | 3.12 ± 0.81 |
| by mice (% of initial) | | | |

### Example 7

### Gene Expression in Lung of Healthy Mice after Aerosol Gene Delivery

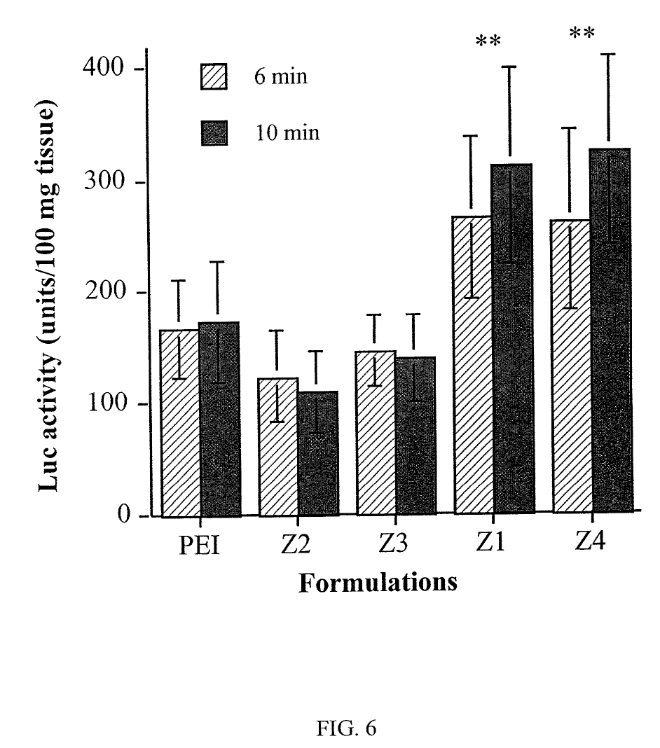

In vivo experiments were used to demonstrate that the formulations containing multiple cationic polymers are more efficient than the formulations containing single cationic polymer in delivering genes to lungs of healthy mice by aerosol administration. The Z1, Z4 (containing multiple cationic polymers), Z2, and Z3 (containing single cationic polymer) were complexed with the reporter luciferase gene using the method described in Example 4. The initial amount of DNA in the nebulizer was 360 μg in 1.2.ml of PBS. The aerosol airflow rate was 4.0 PSI. Female ICR mice (19-21 g) were divided into 12 treatment groups with 3 mice each. The mice were put in the YZ restriction cage for receiving the aerosol. Each formulation was given to two groups of mice who received 6 or 10 minutes aerosol administration, respectively. Forty hours after the administration, mice lungs were resected and homogenized. The luciferase activity per gram of tissue was determined by a luminometer. The data in FIG. 6 is mean one standard deviation from 3 mice of each group.

### Example 8

### Gene Delivery Efficiency and Tissue Distribution of Aerosol Administered Genes in Mice Bearing Lung Cancer

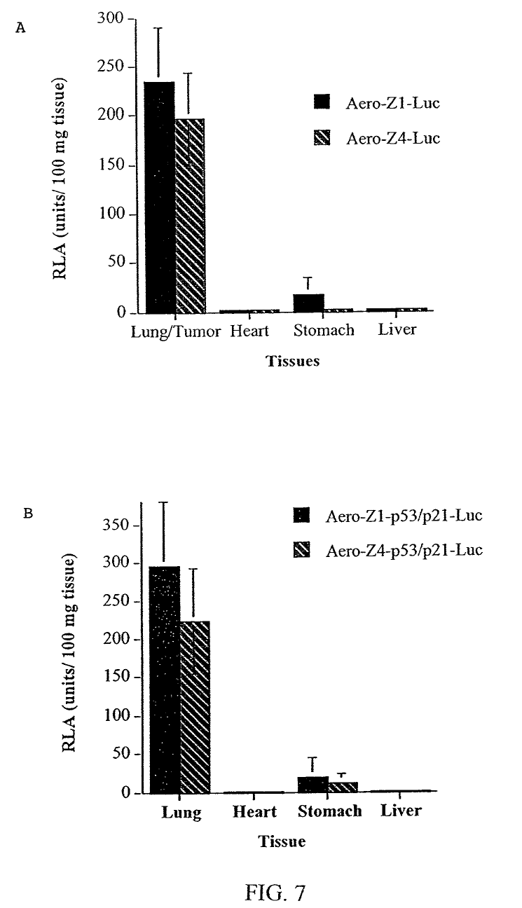

To further confirm the function of the multiple cationic polymers, the mice bearing orthotopic human lung cancer were used for testing the gene delivery efficiency and tissue distribution of the formulations via aerosol administration. In the first experiment, nude mice, 6 to 7 weeks old, were inoculated with the human non-small cell lung carcinoma cell line H358 (2×10⁶ cells/mouse) intratracheally. Seven weeks after the inoculations, the mice were divided into 3 groups with 5 mice each and restricted in the YZ restriction cages before the aerosol administration. The mice were given 10 minutes of aerosol administration of Z1 or Z4 formulation complexed with luciferase expression plasmid (FIG. 7A). The initial amount of formulations in nebulizer was 1.2 ml containing 300 μg of luciferase plasmid. Twenty-four hours after the administration, the organs of mice were resected and the luciferase activity was determined quantitatively. The results in FIG. 7A showed that the formulation containing multiple cationic polymers efficiently and specifically delivered the reporter gene into the lungs and lung tumors of mice via aerosol administration. Little gene expression was found in other tissues. The data is mean ±one standard deviation.

In another experiment, the formulations containing multiple cationic-polymer were complexed with wild-type p53 gene expression plasmid or luciferase expression plasmid driven by p21 promoter (FIG. 7B). The suspension (1.2 ml) was put in the nebulizer reservoir which contained Z1 or Z4 entrapping with 300 μg of CMV promoter driven p53 gene expression plasmid and 300 μg of p21 promoter driven luciferase gene expression plasmid. P53-knockout mice (18-21 g), were given twice 6-minute aerosol administrations with 10-minute interval in YZ restriction cage. Twenty-four hours after the administration the organs of mice were resected. The luciferase levels in the lungs and other organs were determined quantitatively. The data is mean ±one standard deviation from 3 mice. The results in FIG. 7B indicate that because enough amount of wild-type p53 gene and p21 promoter-driven lusiferase gene were delivered and transfected into the lungs of mice, the expressed p53 functioning as a transcription factor was detected in mice.

### Example 9

### Antitumor Activity of Aerosolized Formulations Containing Multiple Cationic Polymers and p53 Gene in Mice Bearing Orthotopic Human Lung Cancer

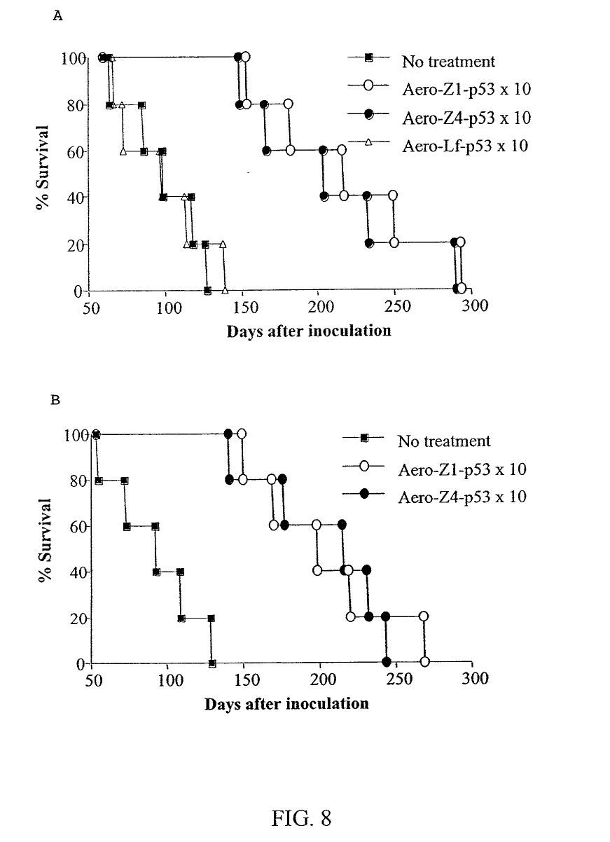

In order to test application potential of the invented formulations, an experimental therapy was designed. Nude mice (7 wks old, 18-20 g) were inoculated with 5×10⁶ human non-small cell lung carcinoma cell lines H358 (FIG. 8A) or H322 (FIG. 8B) intratracheally. The mice in each tumor model were randomly divided into several groups of 5 mice each. Four days after inoculation, the mice in the restriction cages were treated with 10 aerosol administrations of Z1-p53 and Z4-p53, respectively, with 3 days intervals. Each dose was equivalent to 9 μg DNA/mouse. The mice given aerosol G67-liposome-p53 was used as positive control in FIG. 8A and the non treated mice were used as negative control. The survival rate for each group was recorded. This in vivo study demonstrates that the formulations containing of multiple cationic polymers are more efficient than best liposome formulation in delivering the tumor suppressor gene into lungs of mice bearing lung cancer via multiple aerosol administrations. This function resulted in a significant antitumor activity in the mice bearing orthotopic human lung cancer. The data in FIG. 8A and FIG. 8B show that the median survival of mice treated invented formulations carrying p53 was 1.7-fold and 2.3-fold higher than that of the G67-liposome-p53 treated and non-treated in mice, respectively. The data is mean ±one standard deviation from 5 mice.

### Example 10

### Determination of the Subacute Toxicity of formulations Z1-Z5 in mice

The subacute toxicity of formulations Z1, Z2, Z3, Z4 and Z5 was studied in ICR mice after single intratracheal injection. Five different dose levels were used for each formulation. Ten mice were used per dose level. The maximum dose (resulting in 100% animal mortality) and minimum dose (resulting in 100%, animal survival) were selected in preliminary experiments. Animals were observed and weighed daily and animal deaths were recorded. The experiment was terminated on day 14, and the lethal doses, LD₁₀, LD₅₀, LD₉₀, were calculated as described previously (Zou et al., 1995). The results, shown in Table 8 below, indicate the low toxicity of each of the five formulations. The effective dose has been observed to be more than 100 times lower than than LD₁₀.

TABLE 8
Subacute Toxicity after intratracheal injection

| Formulations | LD₁₀ | LD₅₀ | LD₉₀ (mg/kg) |
|---|---|---|---|
| Z1 | 22 ± 8 | 35 ± 12 | 67 ± 25 |
| Z2 | 13 ± 6 | 24 ± 8 | 32 ± 14 |
| Z3 | 89 ± 23 | 147 ± 31 | 232 ± 102 |
| Z4 | 53 ± 17 | 78 ± 22 | 104 ± 45 |
| Z5 | 25 ± 7 | 48 ± 16 | 81 ± 24 |

### XVIII. REFERENCES

The following references, to the extent that they provide exemplary procedural or other details supplementary to those set forth herein, are specifically incorporated herein by reference.

U.S. Pat. No. 3,826,364

U.S. Pat. No. 4,284,412

U.S. Pat. No. 4,498,766

U.S. Pat. No. 4,578,770

U.S. Pat. No. 4,596,792

U.S. Pat. No. 4,599,230

U.S. Pat. No. 4,599,231

U.S. Pat. No. 4,601,903

U.S. Pat. No. 4,608,251

U.S. Pat. No. 4,661,913

U.S. Pat. No. 4,680,338

U.S. Pat. No. 4,714,682

U.S. Pat. No. 4,767,206

U.S. Pat. No. 4,774,189

U.S. Pat. No. 4,857,451

U.S. Pat. No. 4,989,977

U.S. Pat. No. 5,141,648

U.S. Pat. No. 5,160,974

U.S. Pat. No. 5,362,831

U.S. Pat. No. 5,478,722

U.S. Pat. No. 5,563,250

U.S. Pat. No. 5,639,441

U.S. Pat. No. 5,641,662

U.S. Pat. No. 5,744,166

U.S. Pat. No. 5,756,353

U.S. Pat. No. 5,856,456

U.S. Pat. No. 5,880,270

U.S. Pat. No. 5,962,429

U.S. Pat. No. 5,981,501

U.S. Pat. No. 5,985,309

U.S. Pat. No. 6,008,202

U.S. Pat. No. 6,086,913

U.S. Pat. No. 6,090,925

U.S. Pat. No. 6,106,859

Almendro et al., J Immunol., 157(12):541 1-5421, 1996.

Angel et al., Cell, 49:729, 1987b.

Angel et al., Mol. Cell. Biol., 7:2256, 1987a.

Arap et al., Cancer Res., 55:1351-1354,1995.

Atchison and Perry, Cell, 46:253, 1986.

Atchison and Perry, Cell, 48:121, 1987.

Ausubel, et al., In: Molecular Biology. Current Protocols, Greene and Wiley, Harvard Medical School, 1996.

Azuma et al., J Biol Response Mod. 7(5):473-482, 1988.

Bajorin et al., Proc. Annu. Meet. Am. Soc. Clin. Oncol., 7:A967, 1988.

Bakhshi et al., Cell, 41:899, 1985.

Banerji et al., Cell, 27:299, 1981.

Banerji et al., Cell, 35:729, 1983.

Bangham, et al., J. Mol. Biol., 13:238-252, 1965.

Bendas et al., Int. J. Pharm., 181:79 93, 1999.

Berkhout et al., Cell, 59:273, 1989.

Bernard et al., AIDS, 12(16):2125-2139, 1998.

Birchall et al., Int. J. Pharm 197(1-2):221-31, 2000.

Blanar et al., EMBO J, 8:1139, 1989.

Bodine and Ley, EMBO J, 6:2997, 1987.

Boletta et al., Human Gene Therapy, 8:1243-1251, 1997.

Boshart et al., Cell, 41:521, 1985.

Bosze et al., EMBO J, 5:1615, 1986.

Bousiff et al., Proc. Natl. Acad. Sci. USA, 92:7297-7301, 1995.

Boussifet al., Gene Therapy, 3:1074-1080, 1996.

Braddock et al., Cell, 58:269, 1989.

Bulla and Siddiqui, J. Virol., 62:1437, 1986.

Caldas et al., Nat. Genet., 8:27-32,1994.

Campbell and Villarreal, Mol. Cell. Biol., 8:1993, 1988.

Campbell, In: Monoclonal Antibody Technology, Laboratory Techniques in Biochemistry and Molecular Biology, Vol. 13, Burden and Von Knippenberg, Eds. pp. 75-83, Amsterdam, Elseview, 1984.

Campere and Tilghman, Genes and Dev., 3:537, 1989.

Campo et al., Nature, 303:77, 1983.

Canfield et al., Methods Enzymol. 189:418-422, 1990.

Caplen N. J. et al. Nat Med 1: 39-46, 1995.

Caponetti et al, J. Pharm. Sci., 88(1):136-41, 1999.

Carbonelli et al. FEMS Microbiol Lett. 177(1):75-82, 1999.

Celander and Haseltine, J. Virology, 61:269, 1987.

Celander et al., J. Virology, 62:1314, 1988.

Chadwick et al, Gene Ther., 4(9):937-42, 1997.

Chandler et al., Cell, 33:489, 1983.

Chandler et al., Proc Natl Acad Sci USA. 94(8):3596-3601, 1997.

Chang et al., Mol. Cell. Biol., 9:2153, 1989.

Chattejee et al., Proc. Nat'l Acad. Sci. USA., 86:9114, 1989.

Chen et al., Cancer Research, 60(4): 1035-1042, 2000.

Cheng et al, Cancer Res., 54:5547-5551,1994.

Choi et al., Cell, 53:519, 1988.

Cleary et al., Cell, 47(1):19-28, 1986.

Cocea, Biotechniques, 23:814-816, 1997.

Cohen et al., J. Cell. Physiol., 5:75, 1987.

Coll et al., Human Gene Therapy, 10(10):1659-66, 1999.

Costa et al., Mol. Cell. Biol., 8:81, 1988.

Cripe et al., EMBO J., 6:3745, 1987.

Cristiano and Roth, J. Mol. Med., 73:479-486,1995.

Crook et al, Gene Ther., 3(9):834-9, 1996.

Culotta and Hamer, Mol. Cell. Biol., 9:1376, 1989.

Culver et al., Science, 256:1550-1552, 1992.

Dandolo et al., J. Virology, 47:55, 1983.

De Villiers et al., Nature, 312:242, 1984.

Dekie et al., J Control Release 65(1-2):187-202, 2000.

Densmore et al. J Gene Med. 1(4):251-64, 1999.

Densmore et al., Molecular Therapy, 1:180-188, 2000.

Deschamps et al., Science, 230:1174, 1985.

Eastman et al. Hum. Gene Ther. 8(6):765-73, 1997.

Edbrooke et al., Mol. Cell. Biol., 9:1908, 1989.

Edlund et al., Science, 230:912, 1985.

Egholm et al., Nature, 365(6446):566-568, 1993.

el-Gorab, Biochim Biophys Acta. 13:306(1):58-66, 1973.

EP 0273 085

EP 266,032

Felgner et al., Proc. Natl. Acad. Sci. USA, 84(21):7413-7, 1987.

Feng and Holland, Nature, 334:6178, 1988.

Firak and Subramanian, Mol. Cell. Biol., 6:3667, 1986.

Foecking and Hofstetter, Gene, 45:101, 1986.

Fraley et al., Proc Nat'l. Acad. Sci. USA 76:3348-3352, 1979.

Freifelder, Physical Biochemistry, Second Edition, pages 238-246.

Froehler et al., Nucleic Acids Res. 14(13):5399-5407, 1986.

Fronsdal et al., Prostate, 43(2):111-117, 2000.

Fujita et al., Cell, 49:357, 1987.

Gao and Huang, Biochemistry 35: 1027-1036, 1996.

Gao et al., Human Gene Therapy, 4:17-23, 1993.

Gautam et al., Mol Ther. 2(l):63-70, 2000.

Gefter et al., Somatic Cell Genet. 3:231-236, 1977.

Gilles et al., Cell, 33:717, 1983.

Gloss et al., EMBO J., 6:3735, 1987.

Godbey et al. J. Biomed Mater Res. 51(3):321-328, 2000.

Godbout et al., Mol. Cell. Biol., 8:1169, 1988.

Gonda, Critical Reviews in Therapeutic Drug Carrier Systems, 6:273-313, 1990.

Goodbourn and Maniatis, Proc. Nat'l Acad. Sci. USA, 85:1447, 1988.

Goodbourn et al, Cell, 45:601, 1986.

Goula et al., Gene Therapy, 5(9):1291-5,1998.

Greene et al., Immunology Today, 10:272, 1989.

Greenwald, et al., J. Med. Chern., 39:424-431, 1996.

Gregoriadis and Davis, Biochem Biophys Res Commun., 89(4):1287-1293, 1979.

Grosschedl and Baltimore, Cell, 41:885, 1985.

Haensler and Szoka, Bioconj. Chem. 4: 372-79 (1993).

Hart, Expert Opin. Therapeutic Patents, 10(2):199-208, 2000.

Haslinger and Karin, Proc. Nat'l Acad. Sci. USA., 82:8572, 1985.

Hauber and Cullen, J. Virology, 62:673, 1988.

Hen et al., Nature, 321:249, 1986.

Henry, C&E News, September. 18:49-65, 2000.

Hensel et al., Lymphokine Res., 8:347, 1989.

Herr and Clarke, Cell, 45:461, 1986.

Hirano et al., Macromol. Chem., 180:1125-1130, 1979.

Hirochika et al., J. Virol., 61:2599, 1987.

Hirsch et al., Mol. Cell. Biol., 10:1959, 1990.

Hoes et al. J. Controlled Release, 2:205-213, 1985.

Holbrook et al., Virology, 157:211,1987.

Horlick and Benfield, Mol. Cell. Biol., 9:2396, 1989.

Huang et al., Cell, 27:245, 1981.

Hug et al., Mol Cell Biol, 8(8):3065-79, 1988.

Hunter et al., Vaccine. 9(4):250-256, 1991.

Husson et al., J Bacteriol. 172(2):519-524, 1990.

Hussussian et al., Nature Genetics, 15-21, 1994.

Hwang et al., Mol. Cell. Biol., 10:585, 1990.

Irnagawa et al., Cell, 51:251, 1987.

Imbra and Karin, Nature, 323:555, 1986.

Imler et al., Mol. Cell. Biol., 7:2558, 1987.

Inouye et al., Nucl. Acids Res., 13:3101-3109, 1985.

Irie and Morton, Proc. Nat'l Acad. Sci. USA 83:8694-8698, 1986

Jacobs et al., Nature, 327(6122):532-535, 1987.

Jakobovits et al., Mol. Cell. Biol., 8:2555, 1988.

Jameel and Siddiqui, Mol. Cell. Biol., 6:710, 1986.

Jaynes et al., Mol Cell. Biol., 8:62, 1988.

Johnson et al., Mol. Cell. Biol., 9:3393, 1989.

Kadesch and Berg, Mol. Cell. Biol., 6:2593, 1986.

Kafri et al., Proc. Natl. Acad. Sci. USA, 95(19):11377-82, 1998.

Kamb et al., Science, 264:436-440, 1994.

Kamb et al., Nature Genetics, 8:22-26,1994.

Karin et al., Mol. Cell. Biol., 7:606, 1987.

Katinka et al., Cell, 20:393, 1980.

Katinka et al., Nature, 290:720, 1981.

Kato, et al, Cancer Res., 44:25-30, 1984.

Kawamoto et al., Mol. Cell. Biol., 8:267, 1988.

Kerr et al., Br J Cancer., 26(4):239-257, 1972.

Kiledjian et al., Mol. Cell. Biol., 8:145, 1988.

Kircheis et al., Gene Therapy, 4:409-418, 1997.

Klamut et al., Mol. Cell. Biol., 10:193, 1990.

Koch et al., Mol. Cell. Biol., 9:303, 1989.

Kohler and Milstein, Eur. J. Immunol., 6:511-519, 1976.

Kohler and Milstein, Nature, 256:495-497, 1975.

Komberg and Baker, DNA Replication, Second Edition, New York, W. H. Freeman and Company, 1992.

Kraus et al., FEBS Lett, 428(3):165-70, 1998.

Kraus et al., FEBS Lett., 428(3):165-170, 1998.

Kriegler and Botchan, In: Eukaryotic Viral Vectors, Y. Gluzman, ed., Cold Spring Harbor: Cold Spring Harbor Laboratory, NY, 1982.

Kriegler and Botchan, Mol. Cell. Biol., 3:325, 1983.

Kriegler et al., Cell, 38:483, 1984a. Kriegler et al., Cell, 53:45, 1988.

Kriegler et al., In: Cancer Cells 2/Oncogenes and Viral Genes, Van de Woude et al. eds, Cold Spring Harbor, Cold Spring Harbor Laboratory, 1984b.

Kriegler et al., In: Gene Expression, D. Hamer and M. Rosenberg, eds., New York: Alan R. Liss, 1983.

Kuhl et al., Cell, 50:1057, 1987.

Kunzetal., Nucl. Acids Res., 17:1121, 1989.

Lareyrei et al., J Biol Chem, 274(12):8282-90, 1999.

Lareyre et al., J Biol Chem., 274(12):8282-8290, 1999.

Larsen et al., Proc. Nat'l Acad. Sci. USA., 83:8283, 1986.

Laspia etal., Cell, 59:283, 1989.

Latimer et al., Mol. Cell. Biol., 10:760, 1990.

Lee E. R. et al. Hum Gene Ther 7: 1701-1717. 1996.

Lee et al., Artif Organs, 21(9):1002-6, 1997.

Lee et al., Mol. Endocrinol., 2: 404-411, 1988.

Lee et al., Nature, 294:228, 1981.

Lee, Cell Biol, 16(11):1267-75, 1997.

Lee et al., J Auton Nerv Syst., 74(2-3):86-90, 1997.

Lemaitre et al., Proc. Natl. Acad. Sci. USA, 84:648-652, 1987.

Levenson et al., Human Gene Therapy, 9:1233-1236, 1998.

Levinson et al., Nature, 295:79, 1982.

Li, et al., Anti-Cancer Drugs, 7:642-648, 1996.

Lin et al., Mol. Cell. Biol., 10:850, 1990.

Lotte et al., Adv Tuberc Res. 21:107-193, 1984.

Luelno F., “BCG vaccination,” Am Rev Respir Dis. 125(3 Pt 2):70-72, 1982.

Luria et al., EMBO J, 6:3307, 1987.

Lusky and Botchan, Proc. Nat'l Acad. Sci. USA., 83:3609, 1986.

Lusky et al., Mol. Cell. Biol., 3:1108, 1983.

Macejak and Sarnow, Nature, 353:90-94, 1991.

Majors and Varmus, Proc. Nat'l Acad. Sci. USA., 80:5866, 1983.

Marshall, Science, 286(5448):2244-2245, 1999.

Marshall, Science, 286:2244-5, 1999.

Martin et al. Nature, 345(6277):739-743, 1990.

Martin, Journal of Liposome Research, 1(4):407-429, 1990.

Martin, Drugs and the Pharmaceutical Sciences 41267-316, 1990.

McDonald et al. Pharm. Res. 15(5):671-9, 1998.

McNeall et al., Gene, 76:81, 1989.

Miksicek et al., Cell, 46:203, 1986.

Mitchell et al, Proc Natl. Acad. Sci. USA, 90, 11693-11697, 1993.

Mitchell et al., J Clin Oncol. 8(5):856-869, 1990.

Mordacq and Linzer, Genes and Dev., 3:760, 1989.

Moreau et al., Nucl. Acids Res., 9:6047, 1981.

Morimoto, J Pharmacobiodyn. 7(9):688-698, 1984.

Morton and Ravindranath, In Tumor Immunology, Dalgleish (ed.), London: Cambridge University Press, 1-55, 1996.

Morton, et al., Ann. Surg., 216:463-482, 1992.

Muesing et al., Cell, 48:691, 1987.

Nabel et al., Proc Natl Acad Sci USA 90: 11307-11311, 1993.

Ng et al., Nuc. Acids Res., 17:601, 1989.

Nicolau and Sene, Biochem. Biophys. Acta, 721:185-190, 1982.

Nicolau et al., Methods Enzymol., 149:157-176, 1987.

Nobri et al., Nature, 368:753-756,1995.

Nomoto et al., Gene, 236(2):259-71, 1999.

Nomoto et al., Gene, 236(2):259-271, 1999.

Ogris et al. Gene Ther. 6(4):595-605, 1999.

Okamoto et al., Proc. Natl. Acad. Sci. USA, 91:11045-11049,1994.

Ondek et al., EMBO J., 6:1017, 1987.

Omitz et al., Mol. Cell. Biol., 7:3466, 1987.

Palmiter et al., Nature, 300:611, 1982.

PCT/EP/01219

Pech et al., Mol. Cell. Biol., 9:396, 1989.

Pelletier and Sonenberg, Nature, 334:320-325, 1988.

Perales et al., Proc. Natl. Acad. Sci. USA, 91:4086-4090, 1994.

Perez-Stable and Constantini, Mol. Cell. Biol., 10:1116, 1990.

Picard and Schaffner, Nature, 307:83, 1984.

Pinkert et al., Genes and Dev., 1:268, 1987.

Plum et al., Biopolymers, 30:631-643, 1990.

Ponta et al., Proc. Nat'l Acad. Sci. USA., 82:1020, 1985.

Porton et al., Mol. Cell. Biol., 10:1076, 1990.

Queen and Baltimore, Cell, 35:741, 1983.

Quinn et al., Mol. Cell. Biol., 9:4713, 1989.

Rabinovich et al., Science, 265:1401-1402, 1994.

Ravindranath et al., J Immunol Methods. 16;197(1-2):51-67, 1996.

Redondo et al., Science, 247:1225, 1990.

Reisman and Rotter, Mol. Cell. Biol., 9:3571, 1989.

Remington's Pharmaceutical Sciences, 18th Ed. Mack Printing Company, 1990.

Resendez Jr. et al., Mol. Cell. Biol., 8:4579, 1988.

Ripe et al., Mol. Cell. Biol., 9:2224, 1989.

Rittling et al., Nuc. Acids Res., 17:1619, 1989.

Rosen et al., Cell, 41:813, 1988.

Rosenberg et al., Ann Surg. 210(4):474-548, 1989 Rosenberg et al., Human Gene Therapy, 1 1(6):919-79, 2000.

Roth and Cristiano, J Natl. Can. Inst., 89(1):21-39, 1997.

Rudolph et al., J Gene Med., 2(4):269-278, 2000.

Sambrook et al., In: Molecular Cloning: A Laboratory Manual 2 rev.ed., Cold Spring Harbor, Cold Spring Harbor Laboratory Press, 1(77):19-17.29, 1989.

Satake et al., J. Virology, 62:970, 1988.

Schaffner et al., J. Mol. Biol., 201:81, 1988.

Scheit, Nucleotide Analogs, John Wiley, New York, 1980 Searle et al., Mol. Cell. Biol., 5:1480, 1985.

Serrano et al., Nature, 366:704-707,1993.

Serrano et al., Science, 267:249-252,1995.

Sharp and Marciniak, Cell, 59:229, 1989.

Shaul and Ben-Levy, EMBO J., 6:1913, 1987.

Sherman et al., Mol. Cell. Biol., 9:50, 1989.

Sleigh and Lockett, J. EMBO, 4:3831, 1985.

Solodin et al., Biochemistry, 34(41):13537-13544, 1995.

Spalholz et al., Cell, 42:183, 1985.

Spandau and Lee, J. Virology, 62:427, 1988.

Spandidos and Wilkie, EMBO J, 2:1193, 1983.

Stephens and Hentschel, Biochem. J., 248:1, 1987.

Stevenson et al., 1989, J. Gen. Virol., 70:2673-2682.

Stribling et al. Proc Natl. Acad. Sci U.S.A 89(23):11277-81, 1992.

Stuart et al., Nature, 317:828, 1985.

Sullivan and Peterlin, Mol. Cell. Biol., 7:3315, 1987.

Swartzendruber and Lehman, J. Cell. Physiology, 85:179, 1975.

Swift, In Medicine Principles Diagnosis and Therapy (Moren, F. et al. eds) 53-75, 1985.

Szoka et al. Proc. Natl. Acad. Sci., 1978, 75:4194-4198.

Takada et al., Infection and Immunity, 63(1):57-65, 1995a.

Takebe et al., Mol. Cell. Biol., 8:466, 1988.

Tavernier et al., Nature, 301:634, 1983.

Taylor and Kingston, Mol. Cell. Biol., 10:165, 1990a.

Taylor and Kingston, Mol. Cell. Biol., 10:176, 1990b.

Taylor et al., J. Biol. Chem., 264:15160, 1989.

Templeton et al., Nature Biotech., 15(7):647-52, 1997.

Thierry et al., Proc Natl Acad Sci USA. 92(21):9742-9746, 1995.

Thiesen et al., J. Virology, 62:614, 1988.

Tomalia et al., Angew. Chem. Int. Ed. Engl. 29:138-175, 1990.

Treisman, Cell, 42:889, 1985.

Tronche et al., Mol. Biol. Med., 7:173, 1990.

Tronche et al., Mol. Cell. Biol., 9:4759, 1989.

Trudel and Constantini, Genes and Dev., 6:954, 1987.

Tsujimoto and Croce, Proc Natl Acad Sci USA. 83(14):5214-5218, 1986.

Tsujimoto et. al, Science, 229:1390, 19.

Tsumaki, J Biol Chem, 1998 Sep 4;273(36):22861-4, 1998

Tsumaki et al., J Biol Chem. 273(36):22861-22864, 1998.

Tsumaki et al., J Biol Chem. 273(36):22861-22864, 1998.

Tyndall et al., Nuc. Acids. Res., 9:6231, 1981.

van Heeswijk et al., J Controlled Release, 1:301-315, 1985.

Vannice and Levinson, J. Virology, 62:1305, 1988.

Vasseur et al., Proc. Nat'l Acad. Sci. USA., 77:1068, 1980.

Vinogradov et al, Bioconjug. Chem., 9(6):805-12, 1998.

Wagner et al., Science, 260:1510-1513, 1990.

Wagner et al., Proc. Natl. Acad. Sci. 87(9):3410-3414, 1990.

Wang and Calame, Cell, 47:241, 1986.

Weber et al., Cell, 36:983, 1984.

Weinberg, Science, 254:1138-1146, 1991.

Weinberger et al. Mol. Cell. Biol., 8:988, 1984.

Wheeler et al. Proc Natl Acad Sci USA 93: 11454-11459, 1996.

Winoto and Baltimore, Cell, 59:649, 1989.

WO 91/16347

WO90/11092

WO90/11092,

Wong et al., Gene, 10:87-94, 1980.

Wu and Wu, J. Biol. Chem., 262:4429-4432, 1987.

Wu and Wu, Adv. Drug Delivery Rev., 12:159-167, 1993.

Wu et al., Biochem Biophys Res Commun., 233(1):221-6, 1997.

Wu et al., Biochem Biophys Res Commun. 233(1):221-226, 1997.

Xu et al., Gene Therapy, 5:1235-1243, 1998.

Yamamoto et al., Jpn. J Cancer Res., 79:866-873, 1988.

Yang et al., Proc. Natl. Acad. Sci., USA, 91(10):4407-11, 1994.

Yang and Huang, Gene Therapy, 4 (9):950-960, 1997.

Yin et al., J. Biological Response Modifiers, 8:190-205, 1989.

Yutzey et al. Mol. Cell. Biol., 9:1397, 1989.

Yutzey et al. Mol. Cell. Biol., 9:1397, 1989.

Zhao-Emonet, Biochim Biophys Acta, 1442(2-3):109-19, 1998.

Zhao-Emonet et al., Gene Ther. 6(9):1638-1642, 1999.

Zhu et al., Science. 261(5118):209-211, 1993.

Zou et al., Clin Cancer Res. 1(11):1369-74, 1995.

Zou et al., Cancer Gene Ther., 7(5):683-96, 2000.

What is claimed is:

1. A method for administering a composition to a cell, membrane, organ or tissue comprising contacting said composition with said cell, membrane, organ or tissue wherein said composition comprises polyethyleneglycol (PEG), a polycationic polymer, polyethylenimine (PEI) and a cationic lipid.

2. The method of claim 1, wherein said administering occurs as an aerosol.

3. The method of claim 1, wherein said cationic lipid is a diacyl-glycero-ethylphosphocholine.

4. The method of claim 3, wherein said diacyl-glycero-ethylphosphocholine is dipalmitoyl-glycero-ethylphosphocholine (DPEPC).

5. The method of claim 1, wherein said cationic lipid is selected from the group consisting of a diacyl-dimethylammonium propane, a diacyl-trimethylammonium propane dimethyldioctadecylammonium, N-[1-(2,3-ditetradecyloxy)propyl]-N,N-dimethyl-N-hydroxyethylammonium, bromide (DMRIE), N-[1-(2,3,-dioleyloxy)propyl]-N,N-dimethyl-N-hydroxy ethylammonium bromide (DORIE), N-[1-(2,3-dioleyloxy) propyl]-N,N,N-trimethylammonium chloride (DOTMA), DOSPA, 3-beta-[N—(N′,N′-dimethyl-aminoethane)carbamoly]cholesterol (DC-Chol), 3-beta-[N—(N,N-dicarbobenzoxy-spemidine)carbamoyl]cholesterol, and 3-beta-(N-spemine carbamoyl) cholesterol.

6. The method of claim 1, wherein said polycationic polymer comprises polylysine.

7. The method of claim 6, wherein said cationic lipid is a diacyl-glycero-ethylphosphocholine.

8. The method of claim 1, wherein said polycationic polymer comprises protamine.

9. The method of claim 8, wherein said cationic lipid is a diacyl-glycero-ethylphosphocholine.

10. The method of claim 8, wherein said polycationic polymer comprises polylysine.

11. The method of claim 10, wherein said cationic lipid is a diacyl-glycero-ethylphosphocholine.

12. The method of claim 1, wherein said polycationic polymer comprises polylysine.

13. The method of claim 12, wherein said cationic lipid is a diacyl-glycero-ethylphosphocholine.

14. The method of claim 1, further comprising a therapeutic agent wherein the ratio of said composition to said therapeutic agent is no more than about 50:1.

15. The method of claim 14, further comprising a therapeutic agent wherein the ratio of said composition to said therapeutic agent is no more than about 10:1.

16. The method of claim 1, wherein said composition further comprises a nucleic acid.

17. The method of claim 16, wherein said composition further comprises DNA.

18. The method of claim 16, wherein said composition further comprises RNA.

19. The method of claim 1, wherein said composition further comprises a protein.

20. The method of claim 1, wherein said composition further comprises a vaccine.

21. The method of claim 1, wherein said composition further comprises an oligonucleotide.

22. The method of claim 21, wherein said composition further comprises an antisense oligonucleotide.

23. The method of claim 16, wherein said composition further comprises an expression construct.

24. The method of claim 23, wherein said composition further comprises a coding region for p53.

25. The method of claim 1, wherein said composition further comprises a chemical agent.

26. The method of claim 25, wherein said composition further comprises an antibiotic.

27. The method of claim 25, wherein said composition further comprises a chemotherapeutic agent.

28. The method of claim 25, wherein said composition further comprises a diagnostic agent.

29. The method of claim 1, wherein said administering is to the lungs.

30. The method of claim 1, wherein said administering is to the trachea.

31. The method of claim 29, wherein said administering is to the alveoli.

32. The method of claim 29, wherein said composition is administered to prevent or treat lung cancer, a lung infection, asthma, bronchitis, emphysema, bronchilitis, cystic fibrosis, bronchiectasis, pulmonary edema, pulmonary embolism, respiratory failure, pulmonary hypertension, pneumonia or tuberculosis.

33. The method of claim 32, wherein said composition is administered to prevent or treat lung cancer.

34. The method of claim 1, wherein the diameter of particles of said pharmaceutical composition is between 5.0 μm and 0.05 μm.

35. The method of claim 34, wherein the diameter of particles of said pharmaceutical composition is between 0.05 μm and 0.2 μm.

36. The method of claim 1, wherein said composition forms a dry powder.

37. The method of claim 1, wherein said composition forms a liquid.

38. A method for formulating a composition for aerosol delivery comprising combining polyethyleneglycol (PEG), a polycationic polymer, polyethylenimine (PEI) and a cationic lipid to create a composition wherein said composition is capable of being administered as an aerosol.

39. The method of claim 38, wherein said cationic lipid is a diacyl-glycero-ethylphosphocholine.

40. The method of claim 39, wherein said diacyl-glycero-ethylphosphocholine is dipalmitoyl-glyceroethylphosphocholine (DPEPC).

41. The method of claim 38, wherein said cationic lipid is selected from the group consisting of a diacyl-dimethylammonium propane, a diacyl-trimethylammonium propane dimethyldioctadecylammonium, N-[1-(2,3-ditetradecyloxy)propyl]-N,N-dimethyl-N-hydroxyethylammonium, bromide (DMRIE), N-[1-(2,3,-dioleyloxy)propyl]-N,N-dimethyl-N-hydroxy ethylammonium bromide (DORIE), N-[1-(2,3-dioleyloxy) propyl]-N,N,N-trimethylammonium chloride (DOTMA), DOSPA, 3-beta-[N-(N′,N′-dimethyl-aminoethane)carbamoly]cholesterol (DC-Chol), 3-beta-[N-(N,N-dicarbobenzoxy-spemidine)carbamoyl]cholesterol, and 3-beta-(N-spemine carbamoyl) cholesterol.

42. The method of claim 38, wherein said polycationic polymer comprises protamine.

43. The method of claim 42, wherein said cationic lipid comprises a diacyl-glycero-ethylphosphocholine.

44. The method of claim 43, wherein said diacyl-glycero-ethylphosphocholine is dipalmitoyl-glyceroethylphosphocholine (DPEPC).

45. The method of claim 38, wherein said polycationic polymer comprises polylysine.

46. The method of claim 45, wherein said cationic lipid comprises a diacyl-glycero-ethylphosphocholine.

47. The method of claim 46, wherein said diacyl-glycero-ethylphosphocholine is dipalmitoyl-glyceroethylphosphocholine (DPEPC).

48. The method of claim 38, wherein said composition further comprising a stabilizer.

49. The method of claim 38, wherein said composition further comprising a cosolvent.

50. The method of claim 38, wherein said composition further comprises a nucleic acid.

51. The method of claim 50, wherein said composition further comprises DNA.

52. The method of claim 50, wherein said composition further comprises RNA.

53. The method of claim 38, wherein said composition further comprises a protein.

54. The method of claim 38, wherein said composition further comprises a vaccine.

55. The method of claim 38, wherein said composition further comprises an oligonucleotide.

56. The method of claim 55, wherein said composition further comprises an antisense oligonucleotide.

57. The method of claim 50, wherein said composition further comprises an expression construct.

58. The method of claim 57, wherein said composition further comprises a coding region for p53.

59. The method of claim 38, wherein said composition further comprises a chemical agent.

60. The method of claim 59, wherein said composition further comprises an antibiotic.

61. The method of claim 59, wherein said composition further comprises a chemotherapeutic agent.

62. The method of claim 59, wherein said composition further comprises a diagnostic agent.

63. The method of claim 42, wherein the ratio of said PEG to said protamine in said composition is from about 1:1 to 1:5.

64. The method of claim 63, wherein the ratio of said PEG to said protamine in said composition is about 1:2.

65. The method of claim 45, wherein the ratio of said PEG to said polylysine in said composition is from 1:1 to 10:1

66. The method of claim 65, wherein the ratio of said PEG to said polylysine in said composition is about 3:2.

67. The method of claim 38, wherein t he ratio of said PEI to said polycationic polymer in said composition is from about 1:5 to 1:20.

68. The method of claim 67, wherein the ratio of said PEI to said polycationic polymer in said composition is about 1:10.

69. The method of claim 38, wherein the ratio of said cationic lipid to said polycationic polymer in said composition is from about 1:2 to 1:20.

70. The method of claim 40, wherein the ratio of said DPEPC to said polycationic polymer in said composition is from about 1:3 to 1:20.

71. The method of claim 70, wherein the ratio of said DPEPC to said polycationic polymer in said composition is about 1:5.

72. The method of claim 44, wherein the ratio of said components protamine, PEG, PEI, and DPEPC in said composition is from about 2:1:1:0.4 to 50:25:1:10.

73. The method of claim 72, wherein the ratio of said components protamine, PEG, PEI, and DPEPC in said composition is about 10:5:1:2

74. The method of claim 47, wherein the ratio of said components polylysine, PEG, PEI, and DPEPC in said composition is from about 2:3:1:0.4 to 50:80:1:10.

75. The method of claim 74, wherein the ratio of said components polylysine, PEG, PEI, and DPEPC in said composition is about 10:16:1:2.

76. The method of claim 38, wherein the diameter of the particles in said composition is between 10 μm and 0.05 μm.

77. The method of claim 76, wherein the diameter of the particles in said composition is between 0.05 μm and 0.2 μm.

78. The method of claim 38, wherein said composition is formulated as a dry powder.

79. The method of claim 38, wherein said composition is formulated as a liquid.

80. A composition for aerosol delivery comprising polyethyleneglycol (PEG), a polycationic polymer, polyethylenimine (PEI) and a cationic lipid.

81. The method of claim 80, wherein said cationic lipid is a diacyl-glycero-ethylphosphocholine.

82. The method of claim 81, wherein said diacyl-glycero-ethylphosphocholine is dipalmitoyl-glyceroethylphosphocholine (DPEPC).

83. The method of claim 80, wherein said cationic lipid is selected from the group consisting of a diacyl-dimethylammonium propane, a diacyl-trimethylammonium propane dimethyldioctadecylammonium, N-[1-(2,3-ditetradecyloxy)propyl]-N,N-dimethyl-N-hydroxyethylammonium, bromide (DMRIE), N-[1-(2,3,-dioleyloxy)propyl]-N,N-dimethyl-N-hydroxy ethylammonium bromide (DORIE), N-[1-(2,3-dioleyloxy) propyl]-N,N,N-trimethylammonium chloride (DOTMA), DOSPA, 3-beta-[N-(N′,N′-dimethyl-aminoethane)carbamoly]cholesterol (DC-Chol), 3-beta-[N—(N,N-dicarbobenzoxy-spemidine)carbamoyl]cholesterol, and 3-beta-(N-spemine carbamoyl) cholesterol.

84. The method of claim 80, wherein said polycationic polymer comprises protamine.

85. The method of claim 84, wherein said cationic lipid comprises a diacyl-glycero-ethylphosphocholine.

86. The method of claim 85, wherein said diacyl-glycero-ethylphosphocholine is dipalmitoyl-glyceroethylphosphocholine (DPEPC).

87. The method of claim 80, wherein said polycationic polymer comprises polylysine.

88. The method of claim 87, wherein said cationic lipid comprises a diacyl-glycero-ethylphosphocholine.

89. The method of claim 88, wherein said diacyl-glycero-ethylphosphocholine is dipalmitoyl-glyceroethylphosphocholine (DPEPC).

90. The method of claim 80, further comprising a therapeutic agent wherein the ratio of said composition to said therapeutic agent is no more than 50:1.

91. The method of claim 90, further comprising a therapeutic agent wherein the ratio of said composition to said therapeutic agent is no more than 10:1.

92. The method of claim 80, wherein said composition further comprises a nucleic acid.

93. The method of claim 92, wherein said composition further comprises DNA.

94. The method of claim 92, wherein said composition further comprises RNA.

95. The method of claim 80, wherein said composition further comprises a protein.

96. The method of claim 80, wherein said composition further comprises a vaccine.

97. The method of claim 80, wherein said composition further comprises an oligonucleotide.

98. The method of claim 97, wherein said composition further comprises an antisense oligonucleotide.

99. The method of claim 92, wherein said composition further comprises an expression construct.

100. The method of claim 99, wherein said composition further comprises a coding region for p53.

101. The method of claim 80, wherein said composition further comprises a chemical agent.

102. The method of claim 101, wherein said composition further comprises an antibiotic.

103. The method of claim 101, wherein said composition further comprises a chemotherapeutic agent.

104. The method of claim 101, wherein said composition further comprises a diagnostic agent.

105. The method of claim 84, wherein the ratio of said PEG to said protamine in said composition is from about 1:1 to 1:5.

106. The method of claim 105, wherein the ratio of said PEG to said protamine in said composition is about 1:2.

107. The method of claim 87, wherein the ratio of said PEG to said polylysine in said composition is from about 1:1 to 10:1

108. The method of claim 107, wherein the ratio of said PEG to said polylysine in said composition is about 3:2.

109. The method of claim 80, wherein the ratio of said PEI to said polycationic polymer in said composition is from about 1:5 to 1:20.

110. The method of claim 109, wherein the ratio of said PEI to said polycationic polymer in said composition is about 1:10.

111. The method of claim 80, wherein the ratio of said cationic lipid to said polycationic polymer in said composition is from about 1:2 to 1:20.

112. The method of claim 82, wherein the ratio of said DPEPC to said polycationic polymer in said composition is from about 1:3 to 1:20.

113. The method of claim 112, wherein the ratio of said DPEPC to said polycationic polymer in said composition is about 1:5.

114. The method of claim 86, wherein the ratio of said components protamine, PEG, PEI, and DPEPC in said composition is from about 2:1:1:0.4 to 50:25:1:10.

115. The method of claim 114, wherein the ratio of said components protamine, PEG, PEI, and DPEPC in said composition is about 10:5:1:2

116. The method of claim 90, wherein the ratio of said components polylysine, PEG, PEI, and DPEPC in said composition is from about 2:3:1:0.4 to 50:80:1:10.

117. The method of claim 116, wherein the ratio of said components polylysine, PEG, PEI, and DPEPC in said composition is about 10:16:1:2.

118. The composition of claim 80, further comprising an aerosol canister.

119. The composition of claim 80, wherein said aerosol canister comprises a means for metering dosages.

120. A method for reducing toxicity of polyethylenimine (PEI) in an aerosol formulation wherein dipalmitoylglyceroethylphosphocholine (DPEPC) is added to said aerosol formulation in an amount sufficient to reduce the toxicity of said PEI.

121. A composition for aerosol delivery comprising polyethyleneglycol (PEG), a polycationic polymer and a pharmaceutically active agent, wherein the activity of said pharmaceutically active agent ten minutes after aerosolization is at least 50% of initial activity of said pharmaceutically active agent.

122. The composition of claim 121, wherein said activity at ten minutes after aerosolization is at least 60% of initial activity of said pharmaceutically active agent.

123. The composition of claim 122, wherein said activity at ten minutes after aerosolization is at least 70% of initial activity of said pharmaceutically active agent.

124. The composition of claim 121, further comprising polylysine.

125. The composition of claim 121, further comprising protamine.

126. The composition of claim 121, further comprising polylysine and protamine.
